(19) United States
(12) Patent Application Publication
(10) Pub. No.: US 2026/0327711 A1
(43) Pub. Date: Oct. 22, 2026

(54) METHODS AND COMPOSITIONS FOR DIAGNOSING, TREATING, AND/OR PREVENTING INFLAMMATORY BREAST CANCER

(71) Applicant: BOARD OF REGENTS, THE UNIVERSITY OF TEXAS SYSTEM, Austin, TX (US)

(72) Inventors: Alan LAMBOWITZ, Austin, TX (US); Lauren EHRLICH, Austin, TX (US); Elizabeth A.F. KIDDIE, Austin, TX (US); Dennis WYLIE, Austin, TX (US); Hengyi XU, Austin, TX (US); Jun YAO, Austin, TX (US); Naoto UENO, Austin, TX (US); Xiaoping WANG, Honolulu, HI (US)

(21) Appl. No.: 19/486,767

(22) PCT Filed: May 22, 2024
(86) PCT No.: PCT/US2024/030598

§ 371 Date: Nov. 21, 2025

Related U.S. Application Data
(60) Provisional application No. 63/468,115, filed on May 22, 2023.

(51) Int. Cl. C12Q 1/6886

(52) U.S. Cl. CPC C12Q 1/6886; C12Q 2600/118; C12Q 2600/158

(57) ABSTRACT

Disclosed are compositions and methods for diagnosing and treating inflammatory breast cancer. This is done by using a panel of biomarkers.

### CROSS-REFERENCE TO RELATED APPLICATIONS

This application claims benefit of U.S. Provisional Application No. 63/468,115, filed May 22, 2023, incorporated herein by reference in its entirety.

### GOVERNMENT SUPPORT CLAUSE

This invention was made with government support under Grant No. R35 GM136216 and Grant No. R01 CA258523 awarded by the National Institutes of Health. The government has certain rights in the invention.

### BACKGROUND OF THE INVENTION

High-throughput RNA sequencing (RNA-seq) is widely used to identify RNA biomarkers for diseases and environmental stimuli (1). Commonly, an initial comprehensive RNA-seq of one or more RNA biotypes is done to identify candidate biomarkers, which can then be validated by targeted assays in larger numbers of samples. Most desired are biomarkers that can be detected by minimally invasive blood-based assays and reflect molecular aberrations and immune responses throughout the body, with relatively labile blood-based RNA biomarkers potentially advantageous by enabling accurate diagnosis and real-time monitoring of disease progression and response to treatment. Thus far, most studies of cellular and blood-based RNA biomarkers have focused on polyadenylated mRNAs or mature miRNAs, but this approach misses numerous long non-coding RNAs (lncRNAs) and small non-coding RNAs (sncRNAs) as well intron and repeat RNAs that can be detected in blood but have not been widely explored for biomarker potential (2, 3).

Inflammatory breast cancer (IBC) is the most lethal and aggressive type of breast cancer (4). It comprises only 2-4% of breast cancer cases in the United States, but causes 8-10% of breast cancer deaths (5). Due to its high recurrence rate and propensity to metastasize, IBC patients have a shorter median overall survival time than non-inflammatory breast cancer (non-IBC) patients (6-8). Triple-negative IBC patients [TN-IBC, negative for estrogen receptor (ER), progesterone receptor (PR), and human epidermal growth factor receptor-2 (HER2)] have the worst prognosis among the receptor-based molecular subtypes (9). Although a tri-modality treatment (10), consisting of anthracycline-based neoadjuvant chemotherapy, modified radical mastectomy, and postoperative radiation therapy, has substantially improved patient survival outcomes, the median survival of patients with stage III IBC is only 4.75 years (11). FDA-approved targeted therapies specific for IBC are unavailable to date. Precise diagnosis based on molecular biology is the key to developing better treatment for IBC patients.

A number of molecular changes associated with the aggressiveness of IBC have been previously identified, including RNA upregulation of NF-κB gene pathways (12-14); higher RNA expression levels of COX-2 (12, 15), inflammatory cytokines TNFα, IL-6, and IL-8 (16, 17), and the angiogenic factor VEGF (18, 19); protein-level activation of JAK/STAT pathways (20, 21); and elevated protein levels of EGFR (22), E-cadherin (23), Rho-GTPase (24), and translation initiation factor eIF4G1 (25). A 79 gene signature for IBC was identified by integrated analysis of 3 distinct Affymetrix gene expression datasets (26). Additionally, a hybrid epithelial/mesenchymal (E/M) or partial epithelial-to-mesenchymal transition (EMT) phenotype, including both epithelial and mesenchymal features, is often associated with metastatic IBC tumors (27). A particular challenge for IBC studies is that invasive tumors are often distributed as small foci without forming a discrete tumor mass. Thus, it is difficult to obtain high-quality core biopsies with high tumor cellularity, and the rarity of this disease limits patient sample size for optimal analyses. The historical challenges of identifying IBC-specific biomarkers for diagnosis and novel therapeutic targets using tissue-based analyses suggest that blood-based biomarker discovery is critically important for improving the clinical management and survival of IBC patients.

RNA-seq methods used for biomarker identification typically employ engineered retroviral reverse transcriptases (RTs) to convert RNAs into more readily sequenced cDNAs (1, 28). However, retroviral RTs are degenerate enzymes that have inherently low fidelity and processivity, reflecting loss of key regions used by ancestral RTs to bind template RNAs and constrain substrate binding pockets for higher fidelity (29, 30). A series of recent studies have shown that group II intron and other non-LTR-retroelement RTs, evolutionary ancestors of retroviral RTs, can out-perform retroviral RTs for RNA-seq and genome engineering applications (31-36).

Previously, thermostable group II intron-encoded RTs (TGIRTs) were identified from bacterial thermophiles that have higher fidelity, processivity, and strand displacement activity than retroviral RTs, as well as a proficient template-switching activity, which enables efficient seamless RNA-seq adapter addition with small amounts of RNA (31). These properties make it possible for the enzyme to give full-length end-to-end sequence reads of highly modified tRNAs and other structured sncRNAs and enabled the development of TGIRT-seq, a fully validated RNA-seq method that enables comprehensive profiling of coding and non-coding RNAs from small amounts of starting material, including from plasma and extracellular vesicles (2, 3, 37-39).

What is needed in the art are novel biomarkers for inflammatory breast cancer.

### SUMMARY OF THE INVENTION

Disclosed herein are methods of diagnosing and/or treating IBC in a subject. These methods can comprise the steps of: a) obtaining a sample from the subject; b) determining that at least one of the genes or genomic features listed in FIG. 3, 4, 6, 7, or 9 are differentially expressed or present at different levels compared to the same genes or genomic features in a non-IBC control; and c) diagnosing the subject as having IBC. Optionally, the subject can then be treated with appropriate medication specific for IBC.

Likewise, disclosed herein are methods of predicting and treating and/or preventing inflammatory breast cancer (IBC) in a subject, the method comprising the steps of: a) obtaining a sample from the subject; b) determining that at least one of the genes or genomic features listed in FIG. 3, 4, 6, 7, or 9 are differentially regulated compared to the same genes or genomic features in a non-IBC control; c) determining that the subject has an increased risk of having or developing IBC; and d) treating the subject with appropriate medication specific for IBC or referring the subject for additional testing or monitoring.

Further, disclosed are methods of determining the effectiveness of a treatment for inflammatory breast cancer (IBC) in a subject, the method comprising the steps of: a) obtaining a sample from the subject undergoing treatment; b) determining that at least one of the genes or genomic features listed in FIG. 3, 4, 6, 7, or 9 are differentially regulated compared to the same genes or genomic features in a non-IBC control; c) determining how the subject is responding to treatment; and d) adjusting treatment of the subject based on results.

Also disclosed are kits useful in diagnosing IBC. The kits can comprise means for detecting one or more of the genomic features listed in FIG. 3, 4, 6, 7, or 9.

### BRIEF DESCRIPTION OF THE FIGURES

FIG. 1A-B. Principal component analysis and distributions of reads mapped to different RNA biotypes in different sample sets. (A) Principal components analysis (PCA) applied to all genes in all sample sets (left panel) and PCA run separately on genes subsetted by biotype and samples subsetted by sample type (right panel). (B) Stacked bar graphs showing the percentage of reads in each sample assigned to different RNA biotypes (Ensembl GRCh38 Release 93) (top panel: all biotype categories excluding rRNA; middle panel: sncRNA categories; bottom panel: Repeat element categories). Sample tissue type (Frozen, FFPE, PBMC, or Plasma) and disease status (Healthy, non-IBC, or IBC) are indicated above, and sample names corresponding to healthy donor or patient ID numbers are indicated below (H, healthy; B, non-IBC; I, IBC). Paired Wilcoxon sign rank test results for differences in biotype read distributions between sample types can be found in the Supplemental file.

FIG. 2A-D. Pathway analysis and categories of protein-coding genes and miRNA transcripts identified by TGIRT-seq. (A) Statistical significance of enrichment of BROAD Institute's 50 Hallmark gene sets (left panel) or Gene Ontology (GO) gene sets (right panel) in either differentially elevated genes (shades of red or yellow) or depressed genes (shades of blue) for the pairwise comparisons numbered on the right and defined in the key below. Gene-wise differential expression was assessed by DESeq2, with the resulting distribution of test statistics for each gene within each gene set G compared to the distribution of test statistics for all genes not contained within G using a Mann-Whitney U test (MWU). The color of each tile in the heatmap indicates the square-root of-log 10 (p), where p is the MWU enrichment p-value, multiplied by a sign factor of 1 for enrichment in elevated genes and −1 for enrichment in depressed genes. The columns of the heatmap are labeled by the names of the gene sets tested and are clustered to position gene sets with similar MWU significance together. (B) Integrated Genome Viewer (IGV) plots for representative protein-coding genes for which reads mapped largely to exons from mature mRNAs (Type 1, top row) or across the entire gene region including intronic sequences (Type 2, second row). The number of reads for RNAs in each IGV plot is indicated to the right. T and B, top and bottom chromosome strands, respectively. (C) Intron-exon depth ratio (IDR) analysis. Each point indicates the estimated fraction of reads for a protein-coding gene in FFPE IBC tumor samples derived from introns (y-axis, −log (1−y) scaling) compared to the fraction of the genomic annotation of the gene's length corresponding to introns (x-axis, −log (1−x) scaling). Points are colored according to the log 10-transformed length of the genomic interval encoding the gene (exons and introns included): longer genomic intervals tend to be associated with relatively high genomic intron fractions, as is evident from color gradient from left to right. The black line indicates equality y=x, corresponding to the set of points representing genes with IDR=1; points above or below this line represent genes with IDR greater or less than 1, respectively. The red trend line plots the solution to the IDR equation (see Methods) when applied to data for all genes taken together, yielding a typical IDR value of ~0.19 (also the IDR individually assigned to genes represented by points coincident with this line). Trend lines for other IDR values (not shown) would be similarly shaped curves shifted upwards (greater IDR) or downwards (lower IDR). (D) Examples of IGV plots for miRNA genes for which the observed sense-strand reads mapped to either the mature miRNA sequence or longer transcripts within the pre-miRNA sequence, including full-length pre-miRNAs. IGV plots are labeled as in panel B. RNAfold predicted secondary structures of the pre-miRNA hairpins and their calculated minimum free energy values are shown below the corresponding IGV plots. The number of reads is indicated to the right.

FIG. 3. Heat map of expression levels of significantly differentially expressed genes and other notable genes in healthy or tumor breast tissue samples. The color of the tile for gene g in sample s encodes the difference in the (log-transformed DESeq2-normalized) expression level for sample s from overall mean expression for gene g for all samples of same type (frozen tissue or FFPE). The color scale is shown below the heat map. Lanes are numbered according to the patient ID number (Table 1).

FIG. 4A-B. Heat map of expression levels of significantly differentially expressed genes and other notable genes in PBMCs. Expression profiles for (A) select protein-coding genes, lncRNAs, and tRNAs including tRNA halves and 3′-tRFs, and (B) sncRNAs, miRNAs, or Repeat-element RNAs in the PMBC sample set. Tiles are colored by expression difference relative to genewise mean as indicated in the legend of FIG. 3, but with gene-wise mean values taken across all PBMC samples. The color scale is shown below the heat map of panel A. Lanes are numbered according to the healthy donor or patient ID number (Table S1). miRNAs mapping to the hairpin miRNA reference were categorized as: m, mature miRNAs, if reads were 1 or 2 nucleotides shorter or longer than the annotated mature miRNA sequence; pre, pre-miRNAs if reads were derived from pre-miRNA only; or m+pre, if reads were derived from both mature and pre-miRNAs.

FIG. 5. CIBERSORTx-estimated fractions of PBMC cell types for all samples grouped by diagnosis. Points represent individual sample cell type fraction estimates (y-axis) separated by patient diagnosis (Healthy, non-IBC, or IBC). Midpoints of boxes indicate median values for each sample group, with bottom and top of boxes indicating 25th and 75th percentile estimates for within-group distributions of cell type fractions.

FIG. 6A-H. Heatmap of the relative abundance and IGV plots for select genes in the plasma sample set. (A) Genes and features of interest, including those identified by DESeq2 analysis as significantly overrepresented in the IBC plasma dataset. Tiles are colored by expression differences relative to the corresponding genewise means taken across all plasma samples. Color scale at bottom right of panel. miRNAs mapping to the hairpin miRNA reference were categorized as: m, mature miRNAs, if reads were 1 or 2 nucleotides shorter or longer than the annotated mature miRNA sequence; pre, pre-miRNAs if reads were derived from pre-miRNA only; or m+pre, if reads were derived from both mature and pre-miRNAs. (B) Heatmap for called peaks in the plasma sample set using MACS2. Tiles are colored by log-transformed difference in RPM-normalized counts from genewise mean across all plasma samples. Color scale at bottom right. Lanes in heat maps are numbered according to the healthy donor or patient ID number (Table S1). (C)-(H) IGV plots for the indicated genes and peaks. T and B, chromosome top and bottom strands, respectively. The number of reads for RNAs in each IGV plot is indicated to the right.

FIG. 7A-E. Heatmap of the relative abundance for genes and features that were significantly over-represented in healthy and/or non-IBC plasma samples relative to the IBC plasma samples. (A-C) heatmaps for the indicated sample and RNA comparisons. Tiles are colored by expression differences relative to the corresponding genewise means taken across all plasma samples. Lanes in heat maps are numbered according to the healthy donor or patient ID number (Table 1). miRNAs mapping to the hairpin miRNA reference were categorized as: m, mature miRNAs, if reads were 1 or 2 nucleotides shorter or longer than the annotated mature miRNA sequence. (D) IGV plots of overrepresented genes in healthy or non-IBC plasma compared to IBC plasma. The number of reads for RNAs in each IGV plot is indicated to the right. T and B, chromosome top and bottom strands, respectively. (E) Stacked bar graphs showing the percentage of bases in reads mapped to coding sequences (CDS), untranslated regions (UTRs), introns, or intergenic regions for all protein-coding genes detected in the indicated plasma samples (left 3 bars) or for significantly over-represented protein-coding genes (p≤0.001, LFC≥2) detected in ≥50% of the indicated plasma samples (right 4 bars).

FIG. 8A-C. Analysis of cellular origin of TRBJ1 RNAs that were over-represented in IBC plasma samples. (A) IGV plots for RNAs mapping to genomic locus encoding TRBJ1-1 through TRBJ1-6 in combined datasets for different samples. The number of reads for RNAs in each IGV plot is indicated to the right. (B) Stacked bar graph showing RPM for spliced mRNA (blue) and unspliced pre-mRNA (red) for TRBJ1-1, TRBJ1-2, and TRBJ1-6 in different sample types. Sequences corresponding to the splice site for TRBJ1-1, TRBJ1-2, or TRBJ1-6 to TRBC1 were extracted and categorized by sample class, sample type, and RNA type (unspliced pre-mRNA or spliced mRNA). Horizontal thin black lines in the bar graphs for plasma delineate unspliced pre-mRNA reads with different sequences. (C). Sequence alignments for the reads encompassing the splice-site (SS) region of TRBJ1-1, TRBJ1-2, or TRBJ1-6 to TRBC1 (5′SS, 3′SS, and intron 5′ GT and 3′ AG in red). The displayed reads are representative abundant RNA fragments from each category for samples in the same disease category and tissue type. All unique RNA fragments detected in plasma datasets with more than 5 reads are shown. Reads containing spliced exons are connected by gray lines.

FIG. 9A-D. Analysis of cellular origin of intron RNAs that are over-represented in IBC plasma samples. (A) Violin plots with overlaid boxplots (bottom/top of boxes indicate 25^{th}/75^{th} percentiles, respectively) summarizing the distribution of log₁₀-transformed IDR values for samples grouped by diagnosis and tissue. (B) Average plasma expression level of each gene plotted against the average FFPE tumor (left two panels) or PBMC (right two panels) expression level of that gene in either non-IBC samples (1st and 3rd panels) or IBC samples (2nd and 4th panels). Each point represents a single gene, colored according to binned IDR values in FFPE tumor or PBMC samples with color codes shown below the plots. Generalized additive model (GAM) trendlines are fit for each group of IDR-binned genes, with 95% confidence intervals based on standard error estimates indicated by the ribbons around the trendlines. The trendlines show clear separation particularly for the region around 5 on the x-axis in each panel, with high-IDR genes (red points and trendline) tending to show higher average plasma expression than lower-IDR genes with similar FFPE tumor or PBMC expression levels. (C) Bar plot of IDR values for FFPE (top) and PBMC (bottom) sample sets, with diagnostic group indicated by bar color, for intron RNA fragments found in plasma. Asterisks denote genes with IDR≥0.5 in IBC tumors and/or PBMCs, separately calculated for each sample type. (D) IGV plots showing representative intron fragments (sense strand only) detected in IBC FFPE tumors, PBMC, and plasma for 2 genes whose intron RNAs were significantly over-represented in IBC plasma. Vertical black lines and boxes indicate UTRs and exons of the gene. Expanded regions at the bottom show examples of tumor and PBMC read coverage for intron RNA fragments found in plasma. The number of reads for RNAs in each IGV plot is indicated to the right.

FIG. 10A-C. Activation of LINE-1 RNAs in IBC tumor and PBMCs and strandedness of LINE-1 RNA reads in plasma. (A) Scatter plots comparing the relative abundance of different families of LINE RNAs detected in breast tissue, PBMC or plasma samples. DESeq2 normalized counts of LINE elements were averaged by tissue type and disease status and log 2 transformed for pairwise comparisons. Different subfamilies of the LINE element are color coded as shown in the top center panel of the Figure. The numbers at the top left of each scatterplot indicate the percentage of LINE RNAs above the diagonal, a measure of over-expression of LINE elements in each comparison. Pearson's (r) and Spearman's (rs) correlation coefficients are shown at the bottom right. (B) Coverage plots of reads mapped to the L1Hs consensus sequence in combined dataset from different tissues. Coverage depths were normalized to the maximum coverage of the sense or antisense reads in each sample type. (C) Box plots comparing the abundance (RPM) of reads mapped to the L1Hs consensus sequence, and the ratio of reads mapped to L1Hs antisense and sense strand in different tissue types (top 3 plots) correlated with a box plot of DESeq2 normalized counts for TP53 in different tissue types (bottom plot). Wilcoxon tests were used to compare different groups of samples *: p≤0.05; **: p≤0.01; ***: p≤0.001; n.s. not significant. Samples are colored by TP53 mutation status in breast tissues (Frozen healthy, frozen non-IBC tumors, FFPE non-IBC, and FFPE IBC tumors). Somatic TP53 mutations were detected by comparing each of the cancer samples with their putatively healthy counterparts (non-IBC frozen tumor tissue vs matched healthy frozen tissue, FFPE non-IBC or IBC vs matched PBMCs; see FIG. S10). Blue: no mutation; orange: mutation in intron or UTR, red: missense or nonsense mutations in the coding sequence. Patient samples in PBMC and plasma box plots were colored based on the TP53 genotype of the corresponding tumor dataset; the two non-IBC PBMC and plasma samples that lacked corresponding tumor samples are shown as open circles. Abbreviation: ASP, LINE-1 antisense promoter.

FIG. 11A-B. Analysis of reads mapping to protein-coding genes. (A) Percentage of reads mapped to the sense or antisense stand of protein-coding genes. (B) Percentage of bases of protein-coding gene reads mapped to coding sequences (CDS), untranslated regions (UTR), introns, or intergenic regions. Reads for protein-coding genes in the cellular RNA samples mapped largely to the sense strand, while protein-coding gene reads in plasma samples had higher proportions mapping to introns and the antisense strand. Reads mapping to intergenic regions were negligible in all samples. Sample tissue type (Frozen, FFPE, PBMC, or Plasma) and IBC status (Healthy, non-IBC, or IBC) are indicated above, and sample names corresponding to healthy donor or patient ID numbers are indicated below (H, healthy; B, non-IBC; I, IBC).

FIG. 12. Volcano plots for DESeq2 comparisons of FFPE breast tissue samples. The y-axis indicates the log-transformed adj. p-value while the x-axis indicates the effect size (DESeq2-estimated LFC). Each point represents one gene with biotypes color coded for those genes with adj. p≤0.001 and |LFC|≥2. Significantly overrepresented and other notable genes that passed filtering steps and were detected in at least 50% of the samples of the group in which they were overrepresented are labeled by their gene symbol. Protein-coding and lncRNA genes with IDR≥0.5 are marked with an asterisk.

FIG. 13A-D. IGV plots of differentially expressed or notable genes in breast tissue samples. (A) protein-coding genes, (B) lncRNAs, (C) snoRNAs, and (D) mature miRNAs. T and B, chromosomal DNA top strand and bottom strands, respectively. The number of reads for RNAs in each IGV plot is indicated to the right. The primary comparison in Results is for FFPE IBC versus FFPE non-IBC tumors. Read counts between FFPE and Frozen tissue samples are not directly comparable.

FIG. 14A-B. Volcano plots for DESeq2 comparisons of PBMC samples. (A) IBC patient vs Healthy donor. (B) IBC vs non-IBC patients. The y-axis indicates log-transformed adj. p while x-axis indicates LFC. Each point represents one gene with biotypes color coded for those genes with adj. p≤0.001 and |LFC|≥2. Significantly overrepresented and other notable genes that passed filtering steps and were detected in at least 50% of the samples of the group in which they were overrepresented are labeled by their gene symbol. Protein-coding genes with IDR≥0.5 are marked with an asterisk as done elsewhere in the text and Figures; lncRNA genes with IDR≥0.5 were not marked with an asterick due to crowding of gene names (see FIG. 4).

FIG. 15A-E. IGV plots of differentially expressed or notable genes in PBMC samples. (A) protein-coding genes, (B) lncRNAs, (C) sncRNAs, (D) mature miRNAs, and (E) hairpin miRNAs. T and B, chromosomal DNA top strand and bottom strands, respectively. The number of reads for RNAs in each IGV plot is indicated to the right.

FIG. 16A-C. Volcano plots for DESeq2 two-group comparisons in plasma samples. The y-axis indicates log-transformed adj. p while x-axis indicates LFC. Each point represents one gene with biotypes color coded for those genes with adj. p≤0.001 and |LFC|≥2. Significantly overrepresented and other notable genes that passed filtering steps and were detected in at least 50% of the samples of the group in which they were overrepresented are labeled by their gene symbol. (A) shows plasma IBC vs. Healthy+non-IBC. (B) shows plasma IBC vs. Healthy. (C) shows Plasma: IBC vs. non-IBC.

FIG. 17A-D. IGV plots of differentially expressed or notable genes in plasma samples. (A) protein-coding genes, (B) lncRNAs, (C) sncRNAs, and (D) miRNAs. T and B, chromosomal DNA top strand and bottom strands, respectively. The number of reads is indicated to the right.

FIG. 18A-C. IGV plots for protein-coding genes whose intron RNAs were significantly over-represented in IBC plasma and for which intron RNA fragments could be traced to tumors or PBMCs. The IGV plots show coverage in combined datasets for IBC FFPE tumors, PBMC, and plasma with expanded regions at the bottom for intron RNA fragments found in 5 or more IBC plasma samples. The number of reads is indicated to the right.

FIG. 19A-D. Scatter plots comparing the relative abundance of different Repeat-element RNAs detected in breast tissue, PBMC or Plasma. (A) SINE elements, (B) LTR-containing retroelements, (C) DNA elements, and (D) Other repeats. DESeq2-normalized counts of Repeat-elements were averaged by tissue type and disease status and log 2 transformed for pairwise comparisons. The numbers at the top left of each scatterplot indicate the percentage of Repeat-element RNAs above the diagonal, a measure of over-expression of Repeat elements in each comparison. Pearson's (r) and Spearman's (rs) correlation coefficients are shown at the bottom right. Subfamilies of the repeat elements are color coded as indicated in the top center box for each family of repeats.

FIG. 20. Putative mutations in TP53 gene in IBC and non-IBC tumor samples. Putative mutations in the TP53 gene in FFPE and frozen tumor samples were identified by VarScan using the matched PBMC or frozen healthy breast tissue sample of the same patient to distinguish somatic cell mutations from germline mutations or SNPs. The IGV screenshots show the location of each identified mutation in the TP53 gene. Aligned reads from tumor and PBMC or healthy breast tissue of the same patient are divided by a horizontal red line. The number in the lower left corner of each IGV screenshot indicates the patient ID number color coded by the nature of the variation seen in the tumor. SNPs were identified as variations also seen in PBMCs or normal breast tissue from the same patient.

FIG. 21. Scatter plots comparing IBC vs non-IBC DE effect sizes (LFCs) in FFPE tumors and PBMCs. Each point represents an identified overexpressed gene in IBC vs non-IBC patient PBMCs with adj. p≤0.001, LFC≥2 (left panel) or adj. p≤0.05, LFC≥1 (right panel). The y-axis encodes DESeq2 LFC for the FFPE tumor IBC vs non-IBC comparison while the x-axis encodes the LFC for PBMC IBC vs non-IBC comparison. Color indicates gene biotype, as indicated by legend at right. Trendlines indicate the linear model fit summarizing relationship between two comparisons for all plotted genes, fit separately for each indicated gene biotype.

### DETAILED DESCRIPTION OF THE INVENTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a,” “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce,” or “abrogate,” (used interchangeably) or other forms of the word, such as “reducing” or “reduction,” or “abrogating” or “abrogation” is meant lowering of an event or characteristic. It is understood that this is typically in relation to some standard or expected value, in other words it is relative, but that it is not always necessary for the standard or relative value to be referred to.

By “increase” or other forms of the word, such as “increasing,” is meant raising or elevating. It is understood that this is typically in relation to some standard or expected value, in other words it is relative, but that it is not always necessary for the standard or relative value to be referred to.

“Control” refers to a sample or standard used for comparison with an experimental sample. In some embodiments, the control is a sample obtained from a healthy subject (or a plurality of healthy subjects), such as a subject or subjects not expected or known to have a particular polymorphism. In additional embodiments, the control is a historical control or standard reference value or range of values (such as a previously tested control sample or plurality of such samples), or group of samples that represent baseline or normal values. A positive control can be an established standard that is indicative of a specific methylated nucleotide. In some embodiments a control nucleic acid is one that lacks a particular methylated nucleotide, and is used in assays for comparison with a test nucleic acid, to determine if the test nucleic acid includes the methylated nucleotide.

“Detecting” is used herein to identify the existence, presence, or fact of something. General methods of detecting are known to the skilled artisan and may be supplemented with the protocols and reagents disclosed herein. For example, included herein are methods of detecting a nucleic acid molecule in sample. Detection can include a physical readout, such as fluorescence output.

An “isolated” biological component (such as a nucleic acid molecule) has been substantially separated, produced apart from, or purified away from other biological components. Nucleic acid molecules which have been “isolated” include nucleic acids molecules purified by standard purification methods, as well as those chemically synthesized. Isolated does not require absolute purity and can include nucleic acid molecules that are at least 50% isolated, such as at least 75%, 80%, 90%, 95%, 98%, 99% or even 100% isolated.

A “nucleic acid” is a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered backbones, such as peptide nucleic acids (PNA).

A “polymorphism” is a variation in a gene sequence. The polymorphisms can be those variations (DNA sequence differences, e.g., substitutions, deletions, or insertions) which are generally found between individuals or different ethnic groups and geographic locations which, while having a different sequence, produce functionally equivalent gene products. Typically, the term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

“Probes,” as used herein, refer to short nucleic acid molecules, usually DNA or RNA oligonucleotides, typically of about 3-150 nucleotides in length, and more specifically, about 6-100 nucleotides in length, used to detect the presence of a complementary target nucleic acid strand in a sample. All or a portion of a probe can be annealed to a complementary target nucleic acid strand by nucleic acid hybridization to form a hybrid between the probe and the target DNA strand. The probes are associated with metal nanoclusters. Therefore, nucleic acid-metal nanocluster probes can be used to identify a target nucleic acid molecule, wherein the sequence of the probe is specific for the target nucleic acid molecule, for example so that the probe will hybridize to the target nucleic acid molecule under very high stringency hybridization conditions.

Typically, the nucleic acid portion of the probe includes at least about 6 contiguous nucleotides, such as at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50 contiguous nucleotides, that are complementary to a target nucleic acid molecule, such as 20-70 nucleotides, 20-60 nucleotides, 20-50 nucleotides, 20-40 nucleotides, or 20-30 nucleotides. Probes can also be of a maximum length, for example no more than 20, 25, 25, 40, 50, 75 or 100 nucleotides in length. The specificity of a particular probe typically increases with an increase in the number of complementary nucleotides on the probe.

The probe can also include additional nucleotides that are not complementary to the target nucleic acid molecule. The additional nucleotides can be used, for example, for detection of the probe in a sample. In several embodiments, the probes disclosed herein include a hybridization portion that is complementary to a test nucleic acid sequence, and a nucleation portion (that can associate with metal nanoclusters) or an enhancer portion (that can enhance the fluorescence of metal nanoclusters associated with the nucleation portion). The additional nucleotides can be located 5′ or 3′ of the hybridization nucleotides.

Methods for preparing and using nucleic acid probes are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

A “sample,” such as a biological sample, is a sample obtained from a subject. As used herein, biological samples include all clinical samples useful for detection of a methylated nucleotide, including, but not limited to, cells, tissues, and bodily fluids, such as: blood; derivatives and fractions of blood, such as serum; urine; sputum; or CVS samples. Specific examples include peripheral blood mononuclear cells, and samples obtained directly from a tumor (such as via a biopsy). In a particular example, a sample includes blood obtained from a human subject, such as whole blood or serum.

A “test nucleic acid molecule” refers to a nucleic acid molecule whose detection, quantitation, qualitative detection, characterization, or a combination thereof, is intended. For example, the test nucleic acid molecule can be a defined region or particular portion of a nucleic acid molecule, for example a portion of a genome (such as a gene or a region of DNA or RNA containing a gene or portion thereof of interest). The nucleic acid molecule need not be in a purified form. Various other nucleic acid molecules can also be present with the test nucleic acid molecule. For example, the test nucleic acid molecule can be a specific nucleic acid molecule (which can include RNA or DNA), for which the detection of a particular polymorphism is intended. In some examples, a test nucleic acid includes a viral nucleic acid molecule, or a bacterial nucleic acid molecule. Purification or isolation of the test nucleic acid molecule, if needed, can be conducted by methods known to those in the art, such as by using a commercially available purification kit or the like.

By “contacting” is meant placement in direct physical association, for example solid, liquid or gaseous forms. Contacting includes, for example, direct physical association of fully- and partially-solvated molecules.

As used herein, a “biomarker” is a characteristic that can be objectively measured and evaluated as an indicator of a normal biologic or pathogenic process or pharmacological response to a therapeutic intervention. Biomarker panels and their associated algorithms can encompass one or more analytes (e.g., proteins, nucleic acids, and metabolites), physical measurements, or combinations thereof. As used herein, an “analyte” is a substance or chemical constituent that can be objectively measured and determined in an analytical procedure such as immunoassay or mass spectrometry.

As used herein, “a reference expression level of a biomarker” refers to the expression level of a biomarker established for a subject who has not been diagnosed with IBC. More specifically, this refers to the expression level of a biomarker in a normal/healthy subject who has been determined by one skilled in the art using established methods as described herein to not have IBC, and/or a known expression level of a biomarker obtained from literature. It can also refer to the expression level of a biomarker of a subject who has been diagnosed with non-IBC breast cancer.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, and combinations thereof.

“Signature” refers to the differential expression pattern when looking at gene expression. A signature may be exemplified by a particular set of biomarkers.

A “similarity value” is a number that represents the degree of similarity between two things being compared. For example, a similarity value may be a number that indicates the overall similarity between a cell sample expression profile using specific phenotype-related biomarkers and a control specific to that template (for instance, the similarity to a “deregulated growth factor signaling pathway” template, where the phenotype is deregulated growth factor signaling pathway status). The similarity value may be expressed as a similarity metric, such as a correlation coefficient, or may simply be expressed as the expression level difference, or the aggregate of the expression level differences, between a cell sample expression profile and a baseline template.

As used herein, the terms “measuring expression levels,” “obtaining expression level,” and “detecting an expression level” and the like, includes methods that quantify a gene expression level of, for example, a transcript of a gene, or a protein encoded by a gene, as well as methods that determine whether a gene of interest is expressed at all. Thus, an assay which provides a “yes” or “no” result without necessarily providing quantification, of an amount of expression is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. The genes identified as being differentially expressed may be used in a variety of nucleic acid or protein detection assays to detect or quantify the expression level of a gene or multiple genes in a given sample.

A “patient” can mean either a human or non-human animal, preferably a mammal.

As used herein, “subject”, as refers to an organism or to a cell sample, tissue sample or organ sample derived therefrom, including, for example, cultured cell lines, biopsy, blood sample, or fluid sample. The organism may be an animal, including but not limited to, an animal, such as a cow, a pig, a mouse, a rat, a chicken, a cat, a dog, etc., and is usually a mammal, such as a human.

As used herein, the term “pathway” is intended to mean a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway, and a regulatory pathway. Similarly, a pathway can include a combination of these exemplary pathway types.

As used herein, the terms treatment, treat, or treating refers to a method of reducing the effects of a disease or condition (e.g., IBC) or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

The terms “tumor cell growth” or “tumor cell proliferation” are used herein interchangeably to refer to the increase in number of tumor cells.

The terms “cancer” and “tumor” are used herein interchangeably. The terms “breast cancer” or “breast tumor” refer to or describe the physiological condition in mammals in which a population of cells are characterized by unregulated cell growth that originates in the breast tissue of the mammal. All stages of breast cancer are included, including primary cancer or a secondary (metastatic) lesions thereof. Examples of breast cancer include, but are not limited to, advanced stage breast cancer, inflammatory breast cancer, metastatic reoccurrence, secondary tumors originating from breast cancer, among others. This invention is primarily directed towards inflammatory breast cancer.

The term “subject suffering from breast cancer” refers to a subject that presents one or more symptoms indicative of a breast cancer (e.g., a noticeable lump or mass) and/or metastasis thereof, or has been diagnosed as having breast cancer or metastasis thereof.

The terms “effective amount” or “therapeutically effective amount” refer to an amount sufficient to effect beneficial or desirable biological and/or clinical results.

The term “metastasis” or “secondary tumor” refers to cancer cells that have spread to a secondary site, e.g., outside of the breast tissue. Secondary sites include, but are not limited to, the lymphatic system, skin, distant organs (e.g., liver, stomach, pancreas, brain, etc.) and the like.

### General Description

Inflammatory breast cancer (IBC) is a rare and highly aggressive subtype of advanced breast cancer. The aggressive behavior, resistance to chemotherapy, angiogenesis, and high metastatic potential are key intrinsic characteristics of IBC. Pathogenesis and behavior of IBC are closely related to tumor surrounding inflammatory and immune cells, blood vessels, and extracellular matrix, which are all components of the tumor microenvironment (TME). The communication between intrinsic and extrinsic components of IBC and the abundance of cytokines and chemokines in the TME strongly contribute to the aggressiveness and high angiogenic potential of this tumor. Critical modes of interaction are cytokine-mediated communication and direct intercellular contact between cancer cells and tumor microenvironment with a variety of pathway crosstalk (Dobiasova B, Mego M. Biomarkers for Inflammatory Breast Cancer: Diagnostic and Therapeutic Utility. Breast Cancer (Dove Med Press). 2020 Oct. 14; 12:153-163).

Diagnosing, and treating, inflammatory breast cancer (IBC) has proven challenging. Disclosed herein are novel biomarkers, and combinations of biomarkers, which are useful in diagnosing IBC, as well as in prognosing IBC, and in determining effectiveness of treatment for IBC, among other uses. When used as a diagnostic tool, once proper diagnosis of IBC has been made, methods of treating IBC can be used. When used as a guide to treatment, a patient's response to a given protocol can be assessed. This can be done by monitoring the markers disclosed herein before, during, or after treatment. Methods of treating IBC can differ from methods of treating other forms of breast cancer. Results from assays which make use of the biomarkers disclosed herein can help inform treatment. These methods are discussed in more detail below.

Specifically, disclosed herein are markers of IBC. These markers are genes, genomic features, and/or intron-exon junction RNAs, spliced or unspliced precursor RNAs, or intron RNAs related to said gene or genomic feature listed in any of Tables 3-7. Even more specifically, these markers can include, but are not limited to, ADAMTS6, ADAMTSL1, ADGRV1, ANSK1B, BOLL, C6orf118, CCDC150, CHCHD6, CLSTN2, CMSS1, DGKB, DIRAS3, FAM135B, KCNH5, LUZP2, METTL15, MIOS, NADK2, NLGN1, NUP210L, PHF24, PLVAP, SENP2, SPOCK3, TRBJ1-n, TRBJ1-1, TRBJ1-2, TRBJ1-3, TRBJ1-4, TRBJ1-5, TRBJ1-6, UVRAG, WWP2, and XXYLT1. It is noted that TRBJ1-n is the locus of the gene encoding different joining region (TRBJ1-1, etc). Disclosed herein is the entire locus plus the individual segments of the locus that was detected for TRBJ1-n. The markers can be assayed individually, or 2 or more can be used in combination to diagnose a subject with IBC. Specific methods of using biomarkers are discussed below.

Methods of using genetic analysis for diagnosing IBC can include calculating a risk score. This risk score can comprise cutoff values, which can be used to calculate a score. The score can then be used to determine whether the subject has a IBC, or to determine a classification or “level” of IBC, such as stage. One of skill in the art will appreciate how to determine appropriate cutoff values.

For example, various cutoff values can be used to determine a subject's risk of having, or developing, IBC. These scores can comprise multiple cutoff values, which can then be used to place the subject in a risk category. By way of specific example, a risk score of 1-100 can be used to assess the probability of a subject having IBC. It is noted that these values are exemplary only, and a person of skill in the art could readily determine appropriate risk scores, cutoff values, and risk categories.

Turning again to the example of a risk score of 1-100, a score of 100 can indicate the highest level of probability of a subject having IBC, while a score of 0 can mean that the subject is highly unlikely to have IBC. These risk scores can be used to determine cutoff values, which can then be used to place subjects into risk categories (or levels). Again, by way of example, a score of 25 or less (the first cutoff value) can mean that the subject is not likely to have IBC, and does not require further monitoring, or requires less frequent monitoring than a subject with a higher score. This risk category is therefore considered “low risk.” A score from 25-75 (75 being the next cutoff level) can mean that the subject has an increased risk of having or developing IBC. This risk category can be considered “moderate risk.” A score of higher than 75 (the third cutoff level) can mean that the subject is likely to have or develop IBC, and this risk category is considered “high risk.” These categories can then be used to inform treatment, and the type of treatment can differ from subjects without IBC. Again, the risk levels given herein are exemplary only, as one of skill in the art can readily identify appropriate risk levels and cutoff values.

Also disclosed herein are methods of treating IBC in a subject. These methods can comprise the steps of: a) obtaining a sample from the subject; b) determining that at least one of the genes, genomic features, and/or intron-exon junction RNAs, spliced or unspliced precursor RNAs, or intron RNAs related to said gene or genomic feature listed in any of Tables 3-7 are differentially expressed or present at different levels compared to the same genes or genomic features in a non-IBC control; and c) diagnosing the subject as having IBC; and d) treating the subject with appropriate medication specific for IBC.

Likewise, disclosed herein are methods of predicting and treating and/or preventing inflammatory breast cancer (IBC) in a subject, the method comprising the steps of: a) obtaining a sample from the subject; b) determining that at least one of the genes, genomic features, and/or intron-exon junction RNAs, spliced or unspliced precursor RNAs, or intron RNAs related to said gene or genomic feature listed in any of Tables 3-7 are differentially regulated compared to the same genes or genomic features in a non-IBC control; c) determining that the subject has an increased risk of having or developing IBC; and d) treating the subject with appropriate medication specific for IBC or referring the subject for additional testing or monitoring.

Further, disclosed are methods of determining the effectiveness of a treatment for inflammatory breast cancer (IBC) in a subject, the method comprising the steps of: a) obtaining a sample from the subject undergoing treatment; b) determining that at least one of the genes, genomic features, and/or intron-exon junction RNAs, spliced or unspliced precursor RNAs, or intron RNAs related to said gene or genomic feature listed in any of Tables 3-7 are differentially regulated compared to the same genes or genomic features in a non-IBC control; c) determining how the subject is responding to treatment; and d) adjusting treatment of the subject based on results.

Controls are discussed in further detail below. Non-IBC controls can be obtained, for example, from one or more positive, non-IBC breast cancer samples, or from a subject without breast cancer. In addition to IBC status, the control can take into account various other factors, such as HER or ER status, for example. The control can also be from different time points of the patient themselves. For example, a subject can be monitored before, during, or after treatment for IBC. When this is the case, the control can be a comparison to one of these other time points. The gene, or genomic feature, can be upregulated or downregulated compared to a control, or a genomic feature or gene can be present in greater or lesser amounts compared to a control.

The sample from the subject can be a blood, PBMC, tumor, or plasma sample. One of skill in the art will appreciate what type of sample should be used, and how to obtain such a sample.

After the above-described diagnostic test has taken place and the subject's risk score or risk level assessed, the subject can be referred for further monitoring or testing. This further monitoring can include repeating the same diagnostic assay described herein at different time points, or can mean using different tests or different means of monitoring. This additional monitoring can occur at a specified time interval, such as weekly, monthly, biannually, yearly, or every 18 months, 2 years, 3 years, 4 years, 5 years, or longer. This additional testing or monitoring can be carried out to confirm, or to provide further evidence, of, the diagnosis obtained after the initial screen.

For example, the further testing can be carried out to determine that the subject is, indeed, IBC-positive. Further testing can include, but is not limited to, biopsy, imaging, or determining one or more further biomarkers of IBC.

These further biomarkers can comprise, for example, one or more of, on a nucleic acid level: activation of NF-κB pathways; higher expression levels of COX-2; inflammatory cytokines TNFα, IL-6, and/or IL-8; angiogenic factor VEGF; 1; or, on a protein level: activation of JAK/STAT pathways; elevated levels of EGFR, E-cadherin, Rho-GTPase, or translation initiation factor eIF4G1; or a previously identified gene signature for IBC; or a hybrid epithelial/mesenchymal (E/M) or partial epithelial-to-mesenchymal transition (EMT) phenotype, including both epithelial and mesenchymal properties; and/or identifying one or more of ARPC5L, HYOU1, AVEN, CHST11, TLN2, CHST3, PRKCA, COL4A4, or TNC.

Examples of other biomarkers can be found throughout the prior art. Examples include, but are not limited to, those found in the following references, which are hereby incorporated by reference herein in their entirety for their teaching concerning IBC markers: Dobiasova B, Mego M. Biomarkers for Inflammatory Breast Cancer: Diagnostic and Therapeutic Utility. Breast Cancer (Dove Med Press). 2020 Oct. 14; 12:153-163; Lv Q, Liu Y, Huang H, Zhu M, Wu J, Meng D. Identification of Potential Key Genes and Pathways for Inflammatory Breast Cancer Based on GEO and TCGA Databases. Onco Targets Ther. 2020 Jun. 12; 13:5541-5550; and Chakraborty P, George J T, Woodward W A, Levine H, Jolly M K. Gene expression profiles of inflammatory breast cancer reveal high heterogeneity across the epithelial-hybrid-mesenchymal spectrum. Transl Oncol. 2021 April; 14(4):101026.

Various other factors can also be taken into account when determining a risk score or risk level. For example, the subject's past history, family history, or results of other genetic panels.

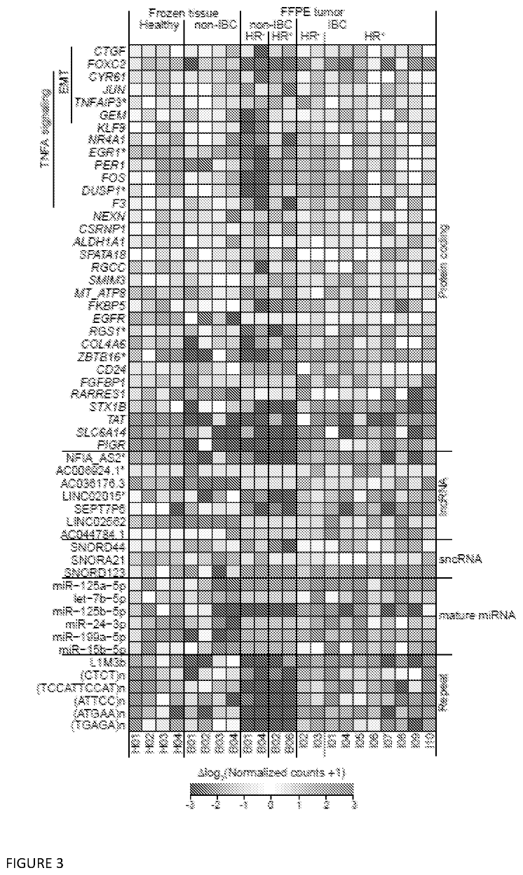

As discussed above, the genes, genomic features, and/or intron-exon junction RNAs, spliced or unspliced precursor RNAs, or intron RNAs related to said gene or genomic feature listed in any of Tables 3-7 can be used individually or in combination (such as in biomarker panels). When used in combination, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more genes can be analyzed together. These can include any of the markers found in FIGS. 3, 4, 6, 7, and 9, or can include additional markers not disclosed herein. By “analyzed together” is meant that their expression can be considered along with the expression of other genes to form a signature, profile, or score, as discussed below.

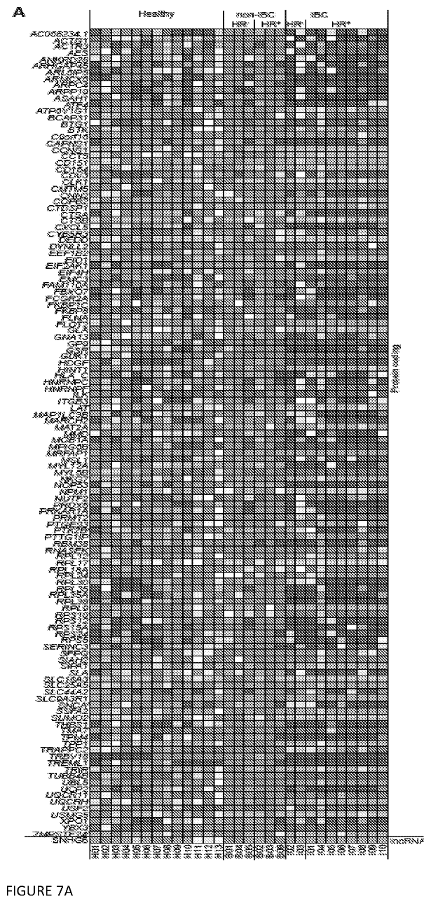

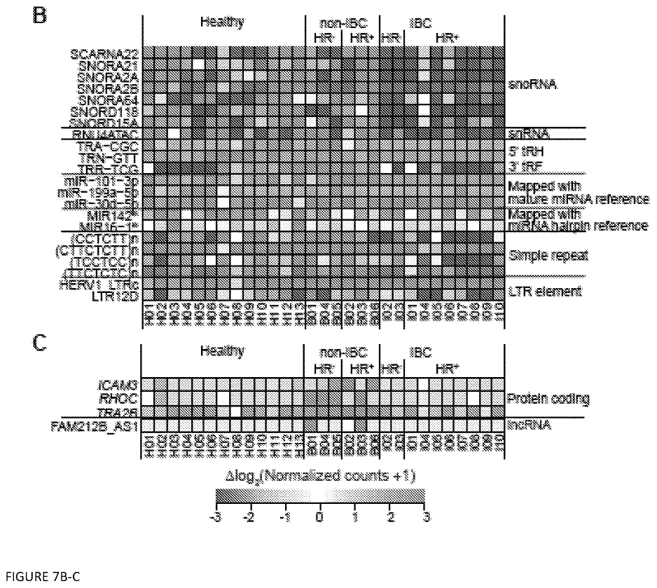

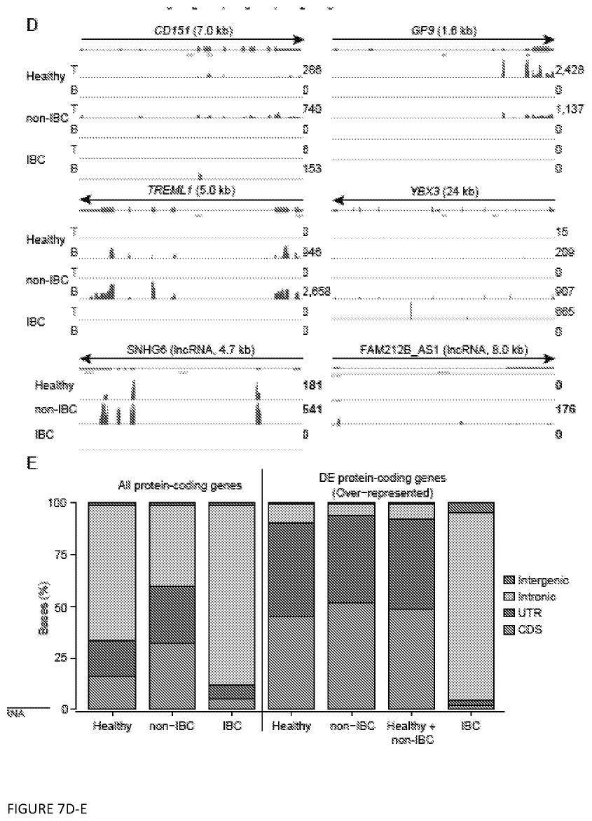

The genes, genomic features, and/or intron-exon junction RNAs, spliced or unspliced precursor RNAs, or intron RNAs related to said gene or genomic feature listed in any of Tables 3-7 can differ between IBC-positive and non-IBC subjects. Particularly noted is that the genes in FIG. 7C can be markers of non-IBC, but can be used to determine the likelihood that a subject has IBC. For example, the markers can be upregulated or downregulated. By “upregulated” means that the marker is expressed or detected in 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 1.1, 1.2, 1.3, 1.4, 1.5, 1.6, 1.7, 1.8, 1.9, 2, 3, 4, 5, 6, 7, 8, 9, or 10 times or more compared to a control.

By “downregulated” means that the marker is expressed or detected 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 1.1, 1.2, 1.3, 1.4, 1.5, 1.6, 1.7, 1.8, 1.9, 2, 3, 4, 5, 6, 7, 8, 9, or 10 times or less compared to a control.

Also disclosed are pathways in which the genes, genomic features, and/or intron-exon junction RNAs, spliced or unspliced precursor RNAs, or intron RNAs related to said gene or genomic feature listed in any of Tables 3-7 are located. Contemplated herein are biomarkers which have been correlated with these pathways via clustering analysis. These biomarkers identified as associating with these specific pathways can also be used as biomarkers of IBC. They can also be used in predicting response of a patient to a compound that modulates the pathway, or measuring pharmacodynamic effect on the pathway of a therapeutic agent. Another aspect of the invention provides a method of using these biomarkers to distinguish severity of IBC in a subject, or in diagnosis of the subject, or to predict response to therapeutic agents. The invention also provides subsets of any of these biomarkers associated with the pathway.

For example, various genes and genomic features disclosed herein can be used to determine status of IBC—e.g., HR+ vs. HR− IBC (GCSHP for example) and or severity of IBC, including high recurrence rate and/or shorter survival time (EGFR and RARRES1 for example). So in addition to diagnosis, various markers disclosed herein can also provide information about severity and subtype of IBC, which can determine that different modes of treatment should be given. (By “different modes” is meant different dosages, timing, or type of medication.)

Any of the sets of biomarkers provided above may be used alone specifically or in combination with biomarkers outside the set. For example, biomarkers that distinguish stage of IBC or risk of developing IBC. Any of the biomarker sets provided above may also be used in combination with other biomarkers disclosed herein, or for any other clinical or physiological condition.

The control can be a standardized gene panel obtained from one or more subjects who had not been diagnosed with IBC when or before control sampling took place. In one example, the control may have a different form of breast cancer. Furthermore, the hormone receptor status of the control and/or the subject can be determined and compared.

The control can also be from the subject being analyzed themselves, and can be from a different time point. For example, the control can be from an earlier time period before treatment began, or can be from a different time point during treatment. For example, the control can be from when the subject was being treated for the disease or disorder, or when the subject was not being treated for the disease or disorder. The control can be used to establish marker levels at different dosages or with various other treatments. One of skill in the art will understand how a control can be used, and varied, to inform a skilled artisan of how a subject is responding to treatment over time, or how a subject's disease or disorder is progressing (or regressing) over time.

According to the present invention, a “baseline” or “control” can include a normal or negative control and/or a disease or positive control, against which a test level of gene expression can be compared. Therefore, it can be determined, based on the control or baseline level of gene expression, whether a sample to be evaluated for IBC has a measurable difference or substantially no difference in gene expression, as compared to the baseline level. In one aspect, the baseline control is indicative of the level of gene expression as expected in a normal (e.g., healthy, negative control, non-IBC) patient. Therefore, the term “negative control” used in reference to a baseline level of gene expression typically refers to a baseline level of expression from a population of individuals which is believed to be normal (i.e., not having or developing IBC). In some embodiments of the invention, it may also be useful to compare the gene expression in a test sample to a baseline that has previously been established from a patient or population of patients with IBC. Such a baseline level, also referred to herein as a “positive control”, refers to a level of gene expression established in a sample from one or preferably a population of individuals who had been positively diagnosed with IBC.

In one embodiment, when the goal is to monitor the progression or regression of IBC in a patient, for example, to monitor the efficacy of treatment of the disease or to determine whether a patient that appears to be predisposed to the disease begins to develop the disease, one baseline control can include the measurements of gene expression in a sample from the patient that was taken from a prior test in the same patient. In this embodiment, a new sample is evaluated periodically (e.g., at annual or more regular physicals), and any changes in gene expression in the patient as compared to the prior measurement and most typically, also with reference to the above-described normal and/or positive controls, are monitored. Monitoring of a patient's gene expression profile tumor can be used by the clinician to prescribe or modify treatment for the patient based on whether any differences in gene expression in the disease or disorder is indicated.

In a preferred embodiment, the control or baseline levels of gene expression are collected from “matched individuals”. According to the present invention, the phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control level of gene expression. Such a form of stored information can include, for example, but is not limited to, a reference chart, listing or electronic file of population or individual data regarding “normal” (negative control) or positive gene expression; a medical chart for the patient recording data from previous evaluations; or any other source of data regarding control gene expression that is useful for the patient to be diagnosed or evaluated.

By “determining stage” is meant determining how far IBC has progressed in the subject. For example, the stage can be determined by the size and location of the primary tumor, the spread of cancer to nearby lymph nodes or other parts of the body, tumor grade, or the presence of certain biomarkers, including those discussed herein.

As discussed in Example 1, RNA levels can be determined using TGIRT analysis. The RNA can be non-coding RNA, such as lncRNA, and can be from an intron. Examples of non-coding RNA include, but are not limited to, transfer RNAs (tRNAs) and ribosomal RNAs (rRNAs), as well as small RNAs such as microRNAs, siRNAs, piRNAs, snoRNAs, snRNAs, exRNAs, scaRNAs, Xist and HOTAIR. Examples of detecting non-coding RNAs can be found, for example, in Beylerli O, Gareev I, Sufianov A, Ilyasova T, Guang Y. Long noncoding RNAs as promising biomarkers in cancer. Noncoding RNA Res. 2022 Feb. 25; 7(2):66-70, herein incorporated by reference in its entirety for its teaching concerning detection of RNA. Biosensors can be used to detect RNA, for example.

Examples of biosensors are known to those of skill in the art. For example, incorporated by reference herein is Mehrotra P. Biosensors and their applications-A review. J Oral Biol Craniofac Res. 2016 May-Augst; 6(2): 153-9. doi: 10.1016/j.jobcr.2015.12.002. Epub 2016 Jan. 6. Biosensors are analytical devices that convert a biological response into an electrical signal. When detecting coding RNA, mRNA levels can be used as a biomarker. The mRNA detection can, for example, comprise reverse-transcription polymerase chain reaction (RT-PCR), quantitative real-time PCR (qRT-PCR), Northern analysis, microarray analysis, and cDNA-mediated annealing, selection, extension, and ligation (DASL) assay (Illumina, Inc.; San Diego, Calif.). Preferably, the RNA detection comprises the cDNA-mediated annealing, selection, extension, and ligation (DASL) assay (Illumina, Inc.). Optionally, the detecting step comprises detecting miRNA levels of the biomarker. The miRNA detection can, for example, comprise miRNA chip analysis, Northern analysis, RNase protection assay, in situ hybridization, miRNA expression profiling panels (Illumina, Inc.), or a modified reverse transcription quantitative real-time polymerase chain reaction assay (qRT-PCR). Preferably the miRNA detection comprises the miRNA expression profiling panels (Illumina, Inc.). Optionally, the detecting step comprises detecting mRNA and miRNA levels of the biomarker(s). The analytical techniques used to determine mRNA and miRNA expression are known. See, e.g., Sambrook et al., Molecular Cloning: A Laboratory Manual, 3^{rd} Ed., Cold Spring Harbor Press, Cold Spring Harbor, N.Y. (2001), Yin et al., Trends Biotechnol. 26:70-6 (2008); Wang and Cheng, Methods Mol. Biol. 414:183-90 (2008); Einat, Methods Mol. Biol. 342:139-57 (2006).

Once diagnosed with IBC, the patient can be treated accordingly. The identification of biomarkers for IBC permits integration of diagnostic procedures and therapeutic regimes. According to Chippa et al., herein incorporated by reference in its entirety regarding treating IBC (Chippa V, Barazi H. Inflammatory Breast Cancer. [Updated 2022 Oct. 31]. In: StatPearls [Internet]. Treasure Island (FL): StatPearls Publishing; 2023), treatment of nonmetastatic inflammatory breast cancer is similar to nonmetastatic noninflammatory locally advanced breast cancer (LABC). The major difference is that sentinel lymph node biopsy and breast conservative therapy are not preferred in IBC even with a very good response to neoadjuvant therapy. The standard therapy includes neoadjuvant chemotherapy, followed by locoregional treatment (grade 1B).

Mastectomy is also an option, such as radical mastectomy with axillary dissection and post-mastectomy radiation. Radiotherapy is also an option. The success of systemic therapy in stage III inflammatory breast cancer can be measured by the pathologic response to the therapy. Anthracycline and taxane-based chemotherapy regimens are commonly used in neoadjuvant therapy. For inflammatory breast cancer with HER-2 overexpression, trastuzumab with or without pertuzumab can be used in addition to neoadjuvant chemotherapy. For hormone receptor-positive, postmenopausal women, aromatase inhibitors like anastrozole can be used. For premenopausal women that are hormone receptor-positive, ovarian function suppression plus tamoxifen can be used.

About one-third of patients with inflammatory breast cancer at the time of diagnosis have stage IV disease. The treatment for metastatic disease is primary systemic therapy to achieve optimal response. Intense locoregional therapy can also be given.

Especially useful biosensors are antibodies. The term ‘antibody’ as used herein may comprise polyclonal, monoclonal, bispecific, humanized or chimeric antibodies, single chain antibodies, Fab fragments and F(ab′)2 fragments, fragments produced by a Fab expression library, anti-idiotypic antibodies and epitope-binding fragments. The term ‘antibody’ also refers to immunoglobulin and T-cell receptor molecules, i.e. molecules that contain an antigen-binding site that specifically binds an antigen. The immunoglobulin molecules can be of any class (e.g. IgA, IgD, IgE, IgG and IgM) or subclasses thereof. If the biomarker is a nucleic acid, a specific probe can be used as ‘biosensor’. Preferably in such a case, the target DNA is first amplified in a PCR reaction with suitable primer sequences.

Appropriate diagnostic tools such as biosensors can be developed in methods and uses of the invention; and detection and quantification of the biomarker can be performed using a biosensor in a microanalytical system, a microengineered system, a microseparation system, an immunochromatography system or other suitable analytical devices (such as Raman or mass spectrography and the like). The biosensor may be incorporated in an immunological method for detection of the biomarker(s), or via electrical, thermal, magnetic, optical (e.g. hologram) or acoustic technologies. Using these techniques, it is possible to detect the target biomarker(s) at the anticipated concentrations found in biological samples. Thus, according to a further aspect of the invention there is provided an apparatus for diagnosing or monitoring IBC.

The biomarker(s) of the invention can be detected using a biosensor incorporating technologies based on “smart” holograms, or high frequency acoustic systems, such systems are particularly amenable to “bar code” or array configurations. In smart hologram sensors (Smart Holograms Ltd, Cambridge, UK), a holographic image is stored in a thin polymer film that is sensitized to react specifically with the biomarker. On exposure, the biomarker reacts with the polymer leading to an alteration in the image displayed by the hologram.

The test result read-out can be a change in the optical brightness, image, color and/or position of the image. For qualitative and semi-quantitative applications, a sensor hologram can be read by eye, thus removing the need for detection equipment. A simple color sensor can be used to read the signal when quantitative measurements are required. Opacity or color of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, methods for detection of one or more biomarkers according to the invention combine biomolecular recognition with appropriate means to convert detection of the presence or quantity of the biomarker in the sample into a signal.

Biosensors to detect one or more biomarkers can also be detected by acoustic, plasmon resonance, holographic and microengineered sensors. Imprinted recognition elements, thin film transistor technology, magnetic acoustic resonator devices and other novel acousto-electrical systems may be employed for detection of the one or more biomarkers of the invention.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the patient's bedside. Suitable platforms for performing methods of the invention include “credit” cards with optical or acoustic readers. The sensor systems can be configured to allow the data collected to be electronically transmitted to the physician for interpretation and thus can form the basis for remote diagnosis.

Methods of the invention can be performed in array format, e.g. on a chip, or as a multi well array. This enables testing for several biomarkers or for only one biomarker in multiple subjects or samples simultaneously. Methods can be adapted into platforms for single tests, or multiple identical or multiple non-identical tests, the last especially in the case if urine-based markers are combined with serum-based markers, and can be performed in high throughput format. Methods of the invention may comprise performing one or more additional, different tests to confirm or exclude diagnosis, and/or to further characterize a condition.

Biomarker-based tests, such as provided by the present invention, provide a first line assessment of ‘new’ patients, and provide objective measures for accurate and rapid diagnosis, in a time frame and with precision, not achievable using the current subjective measures.

Furthermore, diagnostic biomarker tests, such as provided by the present invention, are useful to identify family members or patients at high-risk of developing IBC. This permits initiation of appropriate therapy, or preventive measures, e.g. managing risk factors. These approaches are recognized to improve outcome and may prevent overt onset of the disorder.

Biomarker monitoring methods, biosensors and kits are also vital as patient monitoring tools. If pharmacological treatment is assessed to be inadequate, then therapy can be reinstated or increased; a change in therapy can be given if appropriate. As the biomarkers are sensitive to the state of IBC, they provide an indication of the impact of drug therapy.

Disclosed is a kit for assessing risk level of having or developing IBC, wherein the kit comprises means for detecting two or more the genes, genomic features, and/or intron-exon junction RNAs, spliced or unspliced precursor RNAs, or intron RNAs related to said gene or genomic feature listed in any of Tables 3-7. The means for detection can include a probe or an antibody, or any other suitable means for detection as discussed herein. The kit can further comprise reagents for amplification of the genes, detection of the genes, or both.

It is to be understood that to perform the methods of the present invention, one of skill in the art can make use of any commercially available nucleotide or protein array, wherein hundreds or thousands of genes could be detected if desired. However, in the present method, one would use such an array to selectively screen only for the genes that are described as being useful for detection of IBC as disclosed herein, or to screen for such genes plus any other genes that are known to be useful as a predictor or analysis tool for the same. In addition, the array can be designed to test for more than one disease condition in order to confirm or rule out other potential causes of a patient condition. For example, one may design an assay to screen for the disorders and diseases as described herein, and also secondary indications. In the specifically designed assays described herein, expression of non-informative genes can effectively be “ignored” or not screened. Alternatively, one of skill in the art can prepare nucleotide or protein arrays that are specifically designed to test for the expression of any combination of the genes of interest as described herein, alone or in combination with any other combination of genes that may be useful in evaluating a patient for IBC.

### EXAMPLES

### Example 1: Identification of Inflammatory Breast Cancer Biomarkers by Comprehensive TGIRT-Seq Profiling of RNAs in Tumor and Blood Samples

TGIRT-seq was used to identify potential IBC biomarkers by comprehensively profiling RNAs isolated from Formalin-Fixed Paraffin-Embedded (FFPE) tumor slices, PBMCs, and plasma from IBC and non-IBC patients and healthy donors. This technology identified numerous overexpressed coding and non-coding RNAs in IBC samples of all 3 tissue types, including plasma RNAs that could be traced to tumors and PBMCs as potential proxies for tumor biopsy in diagnosis and real-time monitoring of disease progression and response to treatment.

### Results

### Overview of RNA Samples and TGIRT-Seq Library Preparation

To obtain the 80 samples analyzed in this study, we collected FFPE tumor slices, PBMCs, and plasma from 10 IBC and 4 non-IBC patients plus PBMCs and plasma from 2 additional non-IBC patients and 13 healthy female donors. Additionally, we purchased RNAs isolated from frozen matched tumor and healthy breast tissues from 4 other non-IBC patients (OriGene). Patient information, including hormone receptors (HR) and HER2 status, is summarized in Table 1.

To analyze the samples, we employed a new variation of the TGIRT-seq library preparation method in which RNA extracted from cellular RNA samples was rRNA-depleted and separated into long and short fractions (Zymo RNA Clean & Concentrator kit, two-fraction protocol). The long RNAs (>200 nt) were then chemically fragmented to enable optimal quantitation of mRNAs and lncRNAs (37, 40) and recombined with intact small RNAs (<200 nt) for TGIRT-seq library preparation. Plasma RNA preparations, which contain naturally fragmented mRNAs and largely intact sncRNAs (2, 3), were obtained from 1-2 ng of RNA from 200 ml of plasma and used for TGIRT-seq library preparation directly without rRNA-depletion or size selection. The libraries were sequenced on an Illumina NextSeq to obtain 12.3 to 81.6 million reads per sample.

### Principal Component Analysis

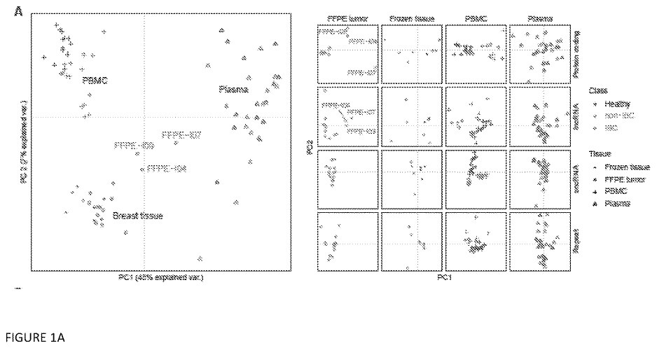

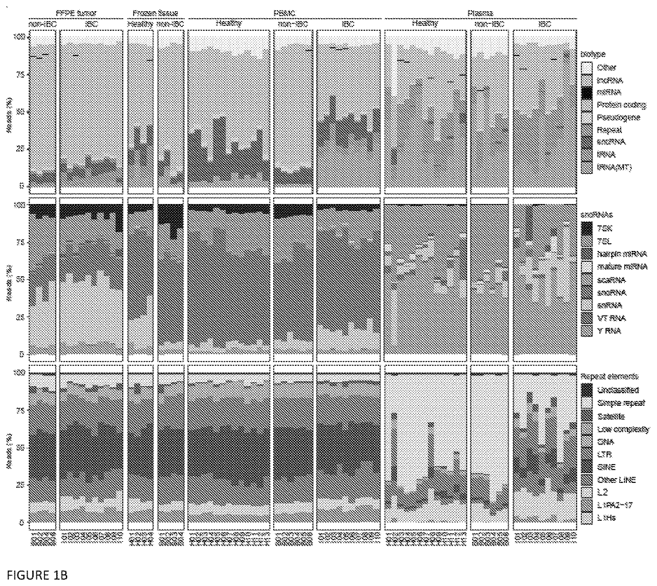

We used principal component analysis (PCA) to assess the degree to which disease state, tissue type, and mode of sample preservation contributed to TGIRT-seq-detected differences between the different samples (FIG. 1A). For all 80 samples considered together, the first principal component (PC1) separated the plasma samples (right) from the tumor and PBMC samples (left), while the second principal component (PC2) split the PBMC samples (+, top left) from frozen tissue and FFPE tumor samples (• and ∘, respectively; bottom left). These findings indicate that sample type was the major source of RNA sequencing differences, as expected.

Within each sample type grouping, the PCA analysis separated the IBC samples (red) from healthy and non-IBC samples (black and blue, respectively), cleanly for the PBMC samples and with some overlap for the breast tissue and plasma samples (FIG. 1A, left), indicating that disease state was a major source of RNA sequencing differences within each sample type. PC1 separated the non-IBC FFPE and frozen samples (blue open and closed circles, respectively), possibly reflecting disease-related differences in the relatively small number of samples and/or differences in the mode of tissue preservation. Perhaps for the latter reasons, 7 of the 10 IBC FFPE tumor samples (red) overlapped 3 of the frozen healthy breast tissue samples (black). The remaining 3 IBC tumor samples (FFPE-104, FFPE-107, and FFPE-109), were outliers with lower gene detection rates than the other FFPE samples; FIG. 1A, left).

Notably, PCAs based on single categories of cellular RNA biotypes (protein-coding gene, lncRNAs, sncRNAs, and RepBase-annotated Repeat RNAs) also largely separated IBC samples (red) from the heathy and non-IBC samples (black and blue, respectively), the major exception being sncRNAs in FFPE IBC and non-IBC tumor slices (FIG. 1A, right), reflecting findings below that only a small number of sncRNAs showed significantly different expression levels between these classes. Collectively, these PCAs indicate that different categories of RNA biotypes, even Repeat RNAs, which are not commonly thought of as a rich source of RNA biomarkers, included sufficient differentially expressed (DE) RNAs to distinguish disease states albeit with lower resolution than simultaneous profiling of multiple RNA biotypes.

### RNA Biotypes Detected by TGIRT-Seq in Different Samples

Stacked bar graphs depicting the proportions of TGIRT-seq reads corresponding to different RNA biotypes (excluding rRNAs) for each of the 80 samples are shown in FIG. 1B.

Reads mapping to protein-coding genes comprised the highest percentages of total reads in both the breast tissue and PBMC samples (31-80%), but lower percentages in plasma samples (3.0-40.6%), where cellular and mitochondrial (MT) tRNAs, sncRNAs, and Repeat RNAs, comprised higher and more variable percentages (FIG. 1B, top panel). 7SL, snoRNAs, and snRNAs comprised the highest percentage of sncRNA reads in the cellular RNA samples, with snoRNAs comprising higher percentages of the sncRNA reads in PBMC samples compared to the other sample types (FIG. 1B, middle panel). Y RNAs comprised the highest percentage of sncRNA reads in most of the plasma samples (FIG. 1B, middle panel). Significant differences in percentages in FIG. 1B were confirmed by paired Wilcoxon sign ranked tests.

The protein-coding gene reads in breast tissue and PBMC samples mapped predominantly (>88%) to the annotated sense strand as expected, while the protein-coding gene reads in plasma included higher proportions mapping to the antisense strand and introns (FIG. 11), in agreement with previous TGIRT-seq analyses of plasma RNAs from healthy individuals (2, 3). Notably, the proportions of reads mapping to intron and antisense RNAs were higher in most of the IBC plasma samples than in the healthy and non-IBC plasma samples (FIG. 11), presaging findings below identifying intron and antisense RNA as potential IBC biomarkers in plasma.

Based on the percentage of total reads, cellular and MT tRNAs appeared to be upregulated relative to other RNA biotypes in IBC compared to non-IBC FFPE tumor samples (Mann-Whitney U (MWU) test p=0.0040), and even more dramatically upregulated in IBC PBMCs (MWU p=1.0e-7, IBC vs combined healthy and non-IBC), likely reflecting PBMC activation, whereas tRNAs appeared to be downregulated in non-IBC PBMCs compared to both healthy and IBC PBMCs (MWU p=5.0e-5 and 8.0e-5, respectively), possibly reflecting immune suppression of PBMC responses (41). The read distribution for the plasma RNA samples was variable irrespective of disease state, with tRNAs being the most variably abundant RNA biotype (FIG. 1B, top panel). This variability may reflect different degrees of cell breakage, despite precautions in the method used for plasma preparation and/or variable degradation of more labile RNA species in clinical plasma samples, which were prepared at different times and used for multiple purposes. Although these caveats should be kept in mind, they did not prevent us from detecting reproducible IBC-specific RNAs in plasma, as described below.

RepBase-annotated repeat RNAs (denoted Repeat-element RNAs) constituted only low proportions of total reads in the cellular tumor and PBMC samples (1.6-7.0% in FFPE tumors, 0.42-1.5% in PBMCs), but much higher proportions of total reads in most of the plasma samples (up to 31%; paired Wilcoxon sign rank test p=6.1e-4 vs FFPE tumors and p=3.7e-9 vs PBMC), indicating preferential export and/or higher stability of these RNAs in plasma (FIG. 1B, top panel). Among the Repeat-element RNAs, simple repeats (mini- and micro-satellites) were more abundant in plasma than in the cellular sample types (FIG. 1B, bottom panel). A benefit of using TGIRT-seq was the technique's ability to simultaneously probe changes in retroelement RNAs, including those from Long Interspersed Nuclear Elements (LINEs), which are highly abundant but normally expressed retrotransposons in the human genome; Short Interspersed Nuclear Elements (SINEs), non-coding RNAs that are mobilized by LINE RTs; and endogenous retroviruses and other LTR-containing retroelement RNAs. Notably, retroelement RNAs comprised higher proportions of the Repeat-element RNAs in most of the IBC plasma samples than in most of the healthy or non-IBC plasma (FIG. 1B, bottom panel; MWU p=1.4e-5 for LINEs and 8.7e-5 for SINEs), reflecting wide-spread over-expression of these elements in IBC (see below).

### TGIRT-Seq Differential Gene Expression (DE) and Intron-Depth Ratio (IDR) Analyses

Previous RNA-seq and microarray studies of protein-coding gene transcripts for both IBC and non-IBC tumor samples have generally focused on mature polyadenylated mRNAs or size-selected mature miRNAs. TGIRT-seq adds multiple dimensions to such analyses by combining better quantitation and 5′ to 3′ coverage of protein-coding genes, lncRNAs, and intron RNAs, with the ability to obtain full-length end-to-end reads of tRNAs and other structured sncRNAs (2, 3, 31, 37, 40).

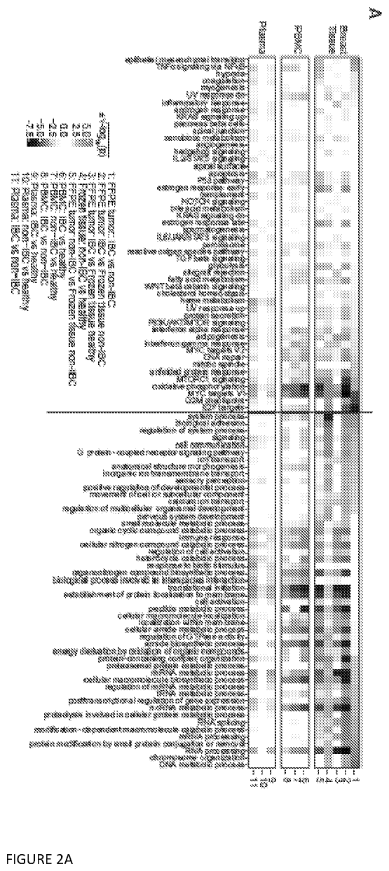

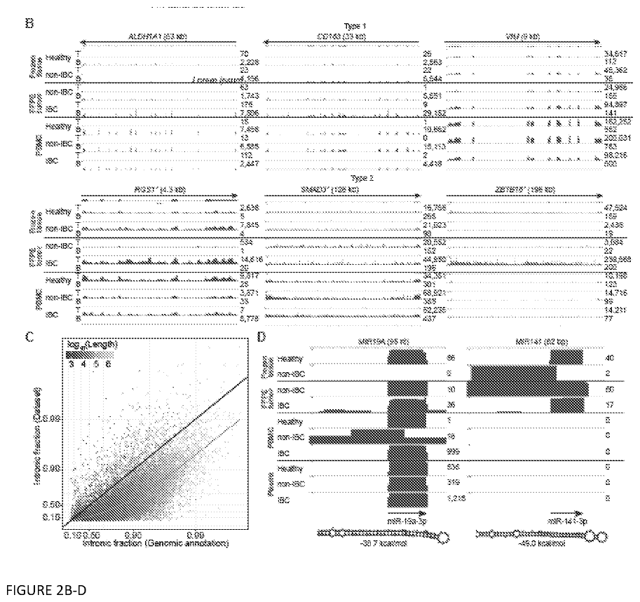

We performed genewise DE analysis by using the DESeq2 R package (42) with size factors calculated based on all protein-coding gene reads in the sample in order to minimize the impact of the tRNA and other sncRNA composition of different sample types on differential expression results. All genes mentioned in the text are listed in the Supplemental file along with their log 2-fold-changes (LFCs), p-values, false discovery rate (FDR) adjusted p-values (written adj. p below), and descriptions of cancer relevance from the literature if available. Unless noted otherwise, the phrase “significantly DE” indicates that the genes in question met the stringent criteria of adj. p≤0.001 and |LFC|≥2 according to DESeq2 analysis. Candidate genes were checked visually by IGV to see if differential expression reflected read coverage across the gene or was limited to specific regions and to filter out protein-coding, lncRNA, and miRNA genes whose differential expression was due to reads corresponding to simple repeats or other low complexity sequences (see Materials and Methods). Hallmark gene set and GO analysis of protein-coding gene transcripts was done by using the GO-MWU method to compare DE statistics for genes included or not included in a given gene set via Mann-Whitney U test (FIG. 2A). Pathways of interest from this analysis are discussed below.

IGV alignments showed that protein-coding gene transcripts detected by TGIRT-seq in cellular RNA samples could be divided into two categories: Type 1 corresponding to mature mRNAs with reads mapping selectively to exons, and Type 2 with reads distributed across exons and introns, most likely derived from unspliced pre-mRNAs, if as in many cases, the reads spanned multiple introns of the same gene (example genes shown in FIG. 2B). Taking advantage of the ability of TGIRT-seq to quantitate intron RNAs, we developed a bioinformatic parameter termed Intron-Exon Depth Ratio (IDR) defined as the average read depth over intronic segments compared to exonic segments, with a threshold IDR of 0.5 chosen to distinguish the Type 1 (IDR<0.5) from Type 2 (IDR≥0.5) gene expression patterns in this study (FIG. 2C). Protein-coding and lncRNA genes with IDR≥0.5 in IBC samples are indicated with an asterisk in the Figures and descriptions. Although the percentage of protein-coding genes exceeding the IDR threshold was roughly equal in the IBC and non-IBC FFPE tumor sample sets (18.4 and 19.5%, respectively; Table 2A), 5 of 28 (17.9%) significantly overexpressed protein-coding genes in IBC vs non-IBC tumors (adj. p≤0.001, LFC≥2) had IDR≥0.5 in IBC, while no such overexpressed protein-coding gene was found in the reciprocal comparison of non-IBC vs IBC (Table 2B). Similarly, 120 of 194 (61.9%) significantly overexpressed protein-coding genes in IBC vs non-IBC PBMCs had IDR≥0.5 in IBC compared to 0 of 3 significantly overexpressed genes with IDR≥0.5 in non-IBC vs IBC PBMCs, with similar trends holding for much larger numbers of overexpressed genes with adj. p≤0.05 and LFC≥1 (Table 2A-C). The higher percentage of overexpressed protein-coding genes with high IDRs in IBC tumors and PBMCs suggests an imbalance between the rates of transcription and RNA splicing and/or intron RNA turnover leading to greater accumulation of unspliced pre-mRNAs and/or excised intron RNAs in IBC.

DE analysis of mature miRNAs and longer transcripts of miRNA loci was done by first mapping reads to the miRBase miRNA hairpin reference followed by remapping to the miRBase miRNA mature reference. DE expressed miRNAs identified by TGIRT-seq included not only mature miRNAs but also longer transcripts, including previously annotated full-length pre-miRNAs (FIG. 2D). For DESeq2 analysis of Repeat-element RNAs, reads mapping to the same repeat element were aggregated by the name of the element and normalized by protein-coding gene reads. In addition to DESeq2, DE RNAs in plasma were also identified by peak calling to identify stable RNA fragments that are resistant to digestion by plasma RNases (3).

Genes were singled out for discussion and inclusion in heatmaps for different sample types according to several criteria: (1) their individual DE statistics; (2) their membership in important biological pathways or gene sets; (3) previous findings of relevance to cancer from the literature; and (4) detection in at least half of the discussed sample group.

### Analysis of Differential Gene Expression in Tumors

We focus first on RNAs detected in tumors. Given the potential issues with differences in tissue preservation methods found in the PCA analysis (FIG. 1A), our primary comparison was IBC vs non-IBC FFPE tumor slices, which is a widely used comparison in the literature (12, 16, 26, 43-45). We then describe trends in other sample comparisons, including between frozen non-IBC tumors and matched frozen healthy breast tissues and between frozen and FFPE non-IBC tumors. For the IBC versus non-IBC FFPE tumor comparison, we identified 43 significantly DE genes (28 protein coding+15 other) with adj. p≤0.001 and |LFC|≥2, with that number increasing to 637 (222 protein coding+415 other) with adj. p≤0.05 and |LFC|≥1.

Gene set analysis performed on the BROAD Institute collection of hallmark gene sets (FIG. 2A left panel) showed upregulation of genes in the TNFα signaling via NFκB (abbreviated TNFα-via-NFκB) and epithelial-mesenchymal transition (EMT) pathways in IBC compared to non-IBC FFPE samples. An NFκB-focused pathway was identified previously as being more highly activated in IBC than non-IBC tumors (46), as was a custom-defined pathway focused on TNFα (26). Recently, the hallmark TNFα-via-NFκB pathway was found to be associated with genome-wide hypertranscription in aggressive tumors of many different cancer types (47, 48), a phenomenon potentially relevant to the prevalence of putative unspliced pre-mRNAs among DE genes in IBC tumors. Genes in BROAD hallmark gene sets E2F targets, G2M checkpoints, and MYC targets V1 were depressed in IBC FFPE compared to non-IBC FFPE tumor samples (FIG. 2A, left panel).

GO-MWU analysis performed on the GO collection of biological process gene sets (FIG. 2A right panel) identified the GO terms system process (GO: 0003008) and biological adhesion (GO: 0022610) as being enriched in genes with elevated expression in IBC relative to non-IBC FFPE tumor samples, and DNA metabolic process (GO: 0006259) as being enriched in genes with depressed expression in IBC compared to non-IBC FFPE tumor samples. The gene set analyzed for the GO term biological adhesion shares 59 genes in common with the 194 genes analyzed from the BROAD hallmark EMT gene set enriched in IBC tumors.

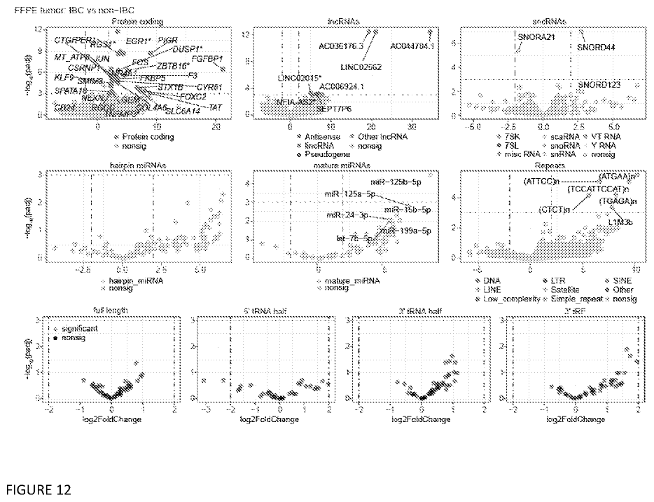

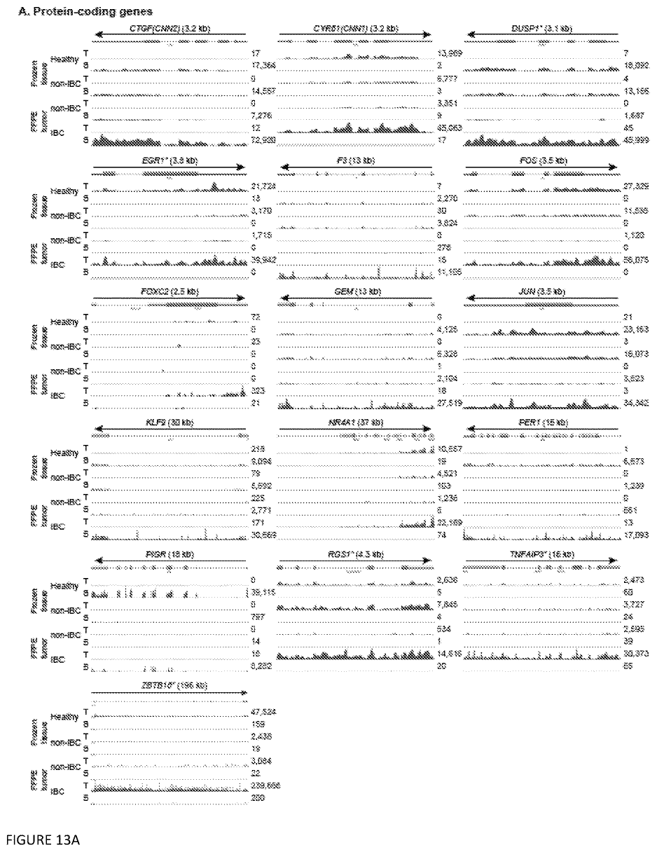

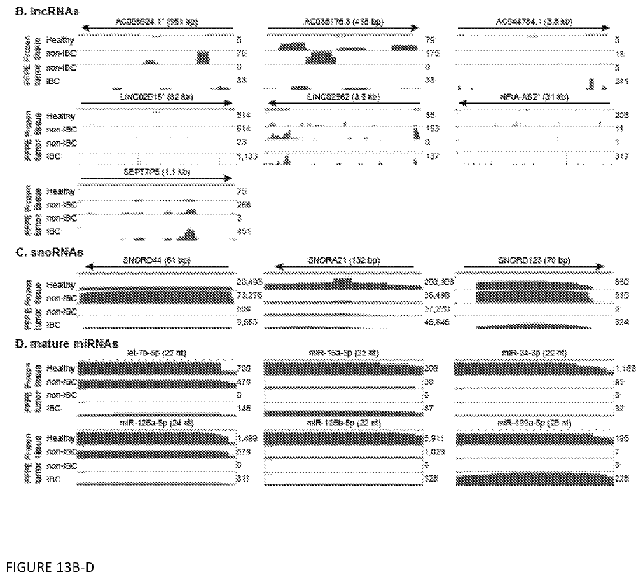

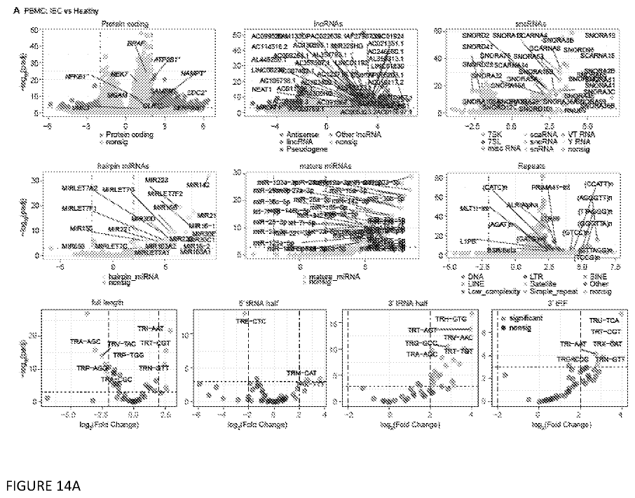

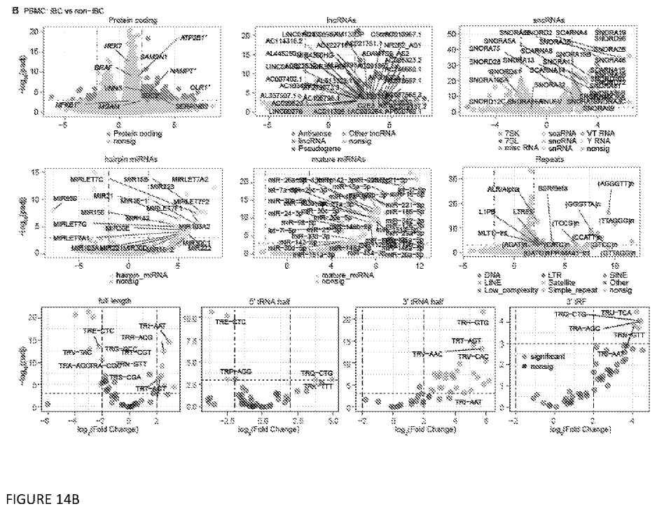

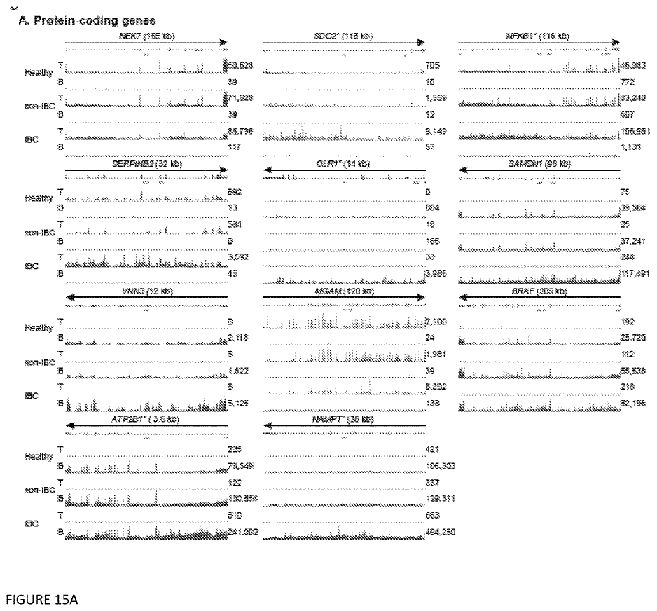

Genes in the EMT pathway that were significantly overexpressed in IBC compared to non-IBC FFPE tumors included JUN, CTGF (aka. CCN2), CYR61 (aka. CCN1), FOXC2, GEM, and TNFAIP3 (heat map FIG. 3, volcano plots FIG. 12, IGV alignments FIG. 13A). Several of these genes—JUN, CYR61, GEM, and TNFAIP3—are also members of the TNFα-via-NFκB pathway. Other genes in the TNFα-via-NFκB set that were significantly overexpressed in IBC included DUSP1*, EGR1*, F3, FOS, PER1, NR4A1, and KLF9. FOS and JUN encode components of Activator Protein 1 (AP-1), a modulator of inflammatory responses in cancers and immune disorders (49).

Three of the 10 most differentially IBC-elevated (by p-value) hallmark TNFα-via-NFκB signaling genes—EGR1*, JUN (also a member of the hallmark EMT gene set), and DUSP1*—were also the 3 most discriminatory genes between IBC and non-IBC samples in a study using an RT-qPCR panel to compare expression of 538 genes in 36 IBC and 22 non-IBC frozen tumor samples (43). EGR1 is a transcriptional regulator that is essential for T and B cell development and mediates an EGFR pathway-regulated immunosuppressive tumor microenvironment in IBC, while DUSP1 promotes tumorigenesis in a number of cancers and regulates immune escape in cancer cells (Supplemental file). Bieche et al. suggested that activation of AP-1, EGR1*, and DUSP1* in IBC may be evidence of intratumoral hypoxia in IBC (43), and we found support for this conjecture in the enrichment of genes with elevated expression in the BROAD hallmark hypoxia gene set in IBC-vs-non-IBC FFPE tumors (FIG. 2A, left panel).

Other significantly DE EMT and/or TNFα-via-NFκB genes included: PER1 consistent with research indicating a link between the disruption of circadian rhythms and the development of breast cancer; F3 (coagulation factor III, aka tissue factor (TF)), whose expression increases in inflammatory and malignant conditions; CTGF/CCN2 and CYR61/CCN1, encoding matricellular proteins involved in the regulation of many biological processes implicated in multiple types of cancer; NR4A, a nuclear transcription factor that is induced by inflammatory cytokines and can potentiate breast cancer cell migration and metastasis; FOXC2, a transcription factor involved in metastasis in breast cancer; and TNFAIP3, which was part of a 6 gene signature for discrimination of IBC from non-IBC by RT-qPCR in a study focusing on NFκB-related genes ((14)). Although not specifically included in the hallmark EMT gene set, PIGR (polymeric immunoglobulin receptor), an inflammatory mediator whose over-expression elicits an EMT in hepatic cell lines, also showed significantly elevated expression in IBC relative to non-IBC tumors (FIG. 3).

Although protein-level activation of the JAK-STAT pathway in IBC patient samples has been reported (20, 21), we did not see much evidence for RNA-level enrichment of the hallmark IL6/JAK/STAT3 signaling gene set in genes with differential expression between IBC and non-IBC FFPE tumor samples (FIG. 2A). The only significantly overexpressed gene (p≤0.001 and LFC≥2) in this gene set in IBC tumors was JUN (FIG. 3), which is also a member of the TNFα-via-NFκB and EMT gene sets (see above). One JAK-STAT gene, IL4R, had adj. p=0.016 and LFC=1.2, while all other members of this gene set, as well as other JAK and STAT genes not included in this gene set had either adj. p>0.05 or LFC<1 for the FFPE IBC vs non-IBC comparison.

Another top DE gene, RGS1* (regulator of G protein signaling 1) exhibited both significantly and consistently higher expression levels in IBC than in non-IBC FFPE tumor samples (FIG. 3 and FIG. 12). RGS1 was identified as a potential biomarker for the progression of tumors in melanoma, with a recent study showing that RGS1 expression in cytotoxic T lymphocytes (CTLs) reduces the trafficking of CTLs to tumors and is associated with shorter survival of patients with breast cancer (Supplemental file).

IBC tumors have also been reported to be enriched in a cancer stem cell (CSC) population defined by protein expression levels as ALDH+ (50) or CD44+CD24−/low (51). RNA expression levels of ALDH1A1 were not significantly elevated in our IBC compared to non-IBC tumor samples (adj. p=0.18, though unadjusted p=0.0074), while CD24 RNA expression levels were moderately decreased (adj. p=0.021; FIG. 3).

EGFR, whose overexpression independently predicts high recurrence rate and shorter survival for IBC patients, and RARRES1 (aka TIG1), whose high expression is correlated with shorter survival times of IBC patients (22), were overexpressed to different degrees in subsets of IBC patient samples, but not significantly when comparing the entire IBC sample set (FIG. 3, adj. p=0.39 and 0.36, respectively). Among other genes or proteins previously noted as elevated in IBC tumors (see Introduction), we found little evidence for differences in RNA expression levels for COX-2 (aka PTGS2), IL6*, IL8 (aka CXCL8), CDH1 (which encodes E-cadherin), EIF4G1, or the various VEGF genes (adj. p>0.05 for all, Supplemental file). One Rho GTPase gene, RHOB, had adj. p=0.022 and LFC=1.7).

Seven lncRNAs were significantly overexpressed in IBC compared to non-IBC FFPE tumor slices (FIG. 12B). Five of these (AC006924.1, AC036176.3, AC044784.1, LINC02562, and SEPT7P6) corresponded to the annotated mature lncRNA, while the other 2 (LINC02015* and antisense RNA NFIA-AS2*) had high coverage across introns (FIG. 13, FIG. 2A). LINC02015 predicts survival in glioblastoma multiform patients and elevated expression of NFIA-AS2 has been correlated with poor prognosis in glioma.

Other significantly overexpressed RNAs in IBC vs non-IBC FFPE tumor samples (FIG. 3 and FIG. 12) included SNORD44, which has an antitumor effect when overexpressed in colorectal cancer cells and showed elevated expression in all 10 of the IBC tumor samples; 5 simple repeat or satellite sequences (ATGAA)n, (ATTCC)n, (CTCT)n, (TGAGA)n, and (TCCATTCCAT)n; and LINE-1 element L1M3b, which has been reported to modulate chromatin assembly (FIG. 3 and FIG. 12). Two other snoRNAs, SNORD123, which was among the key discriminative sncRNAs used in predicting eight different cancer types, and SNORA21, were just below the stringent fold-change or FDR cutoffs (SNORD123: adj. p=0.003, LFC+6.8; SNORA21: adj. p=4.3e-6, LFC-1.8). Despite the elevated expression levels of tRNAs in IBC vs non-IBC FFPE tumors (FIG. 1B, top panel), we found no significant differences in the relative abundance of different full-length tRNA isodecoder species, 5′- or 3′ halves, or 3′ tRFs between these samples at adj. p≤0.001, LFC≥2 (FIG. 12).

We identified one mature miRNA (mir-125b-5p) as significantly upregulated in IBC versus non-IBC FFPE tumor slices, and 5 others (let-7b-5p, mir-125a-5p, mir15b-5p, and mir-199a-5p, and mir-24-3p) as upregulated but with slightly higher adj. p≤0.01 (FIG. 3 and FIG. 12). Although several of these miRNAs were found previously to have cancer relevance, none had been identified previously as upregulated in IBC tumors. Among the 12 miRNAs identified previously as upregulated in IBC tumors (52-55), we found 10 to be nominally elevated (LFCs 0.05 to 3.32) either as mature miRNAs or as fragments derived from pre-miRNA hairpins but adj. p>0.05, while miR-520a-5p appeared down-regulated (adj. p=0.75, LFC-0.95), and we did not detect miR-720 in our datasets.

Comparisons between frozen healthy breast tissue and non-IBC tumor samples for the same set of IBC-focused genes showed mixed results, but with a trend toward lower expression of many of these genes in the frozen non-IBC tumor samples (FIG. 3). While the relatively small number of non-IBC patient samples and their different methods of tissue preservation preclude firm conclusions, these findings suggest that overexpression of some genes in the IBC vs non-IBC FFPE tumors could reflect their downregulation in non-IBC tumors compared to both IBC tumors and healthy breast tissue. Exceptions included TNFAIP3*, GEM, F3, and SMIM3, which had similar expression levels between the paired frozen healthy breast tissue and non-IBC tumor samples (fold changes 0.84-1.16; LFCs=−0.25 to 0.22), and RGS1*, which was among the 10 most significantly elevated genes in the FFPE IBC vs non-IBC comparison (LFC=3.93, adj. p=1.02e-9) and was moderately elevated in the frozen non-IBC tumors vs paired healthy breast tissue (LFC=1.02, adj. p=0.40), indicating up-regulation in IBC tumors.

### Analysis of Differential Gene Expression in PBMCs

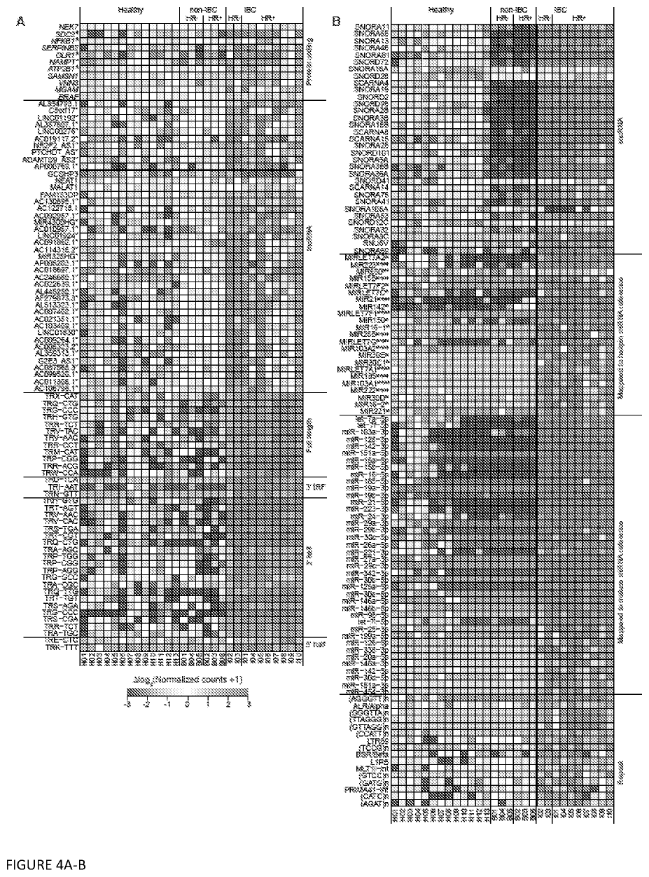

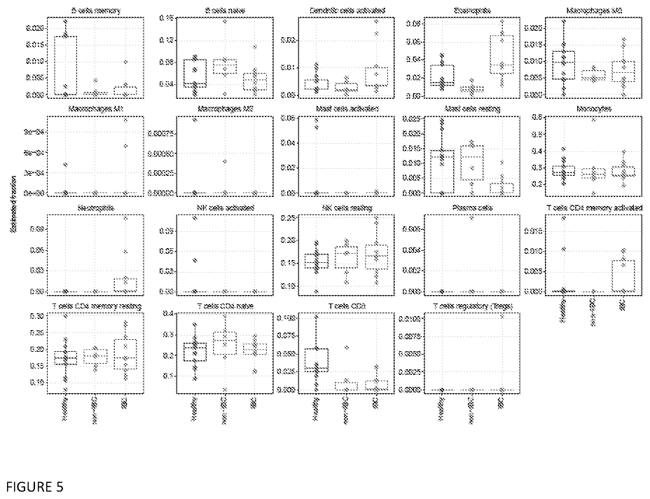

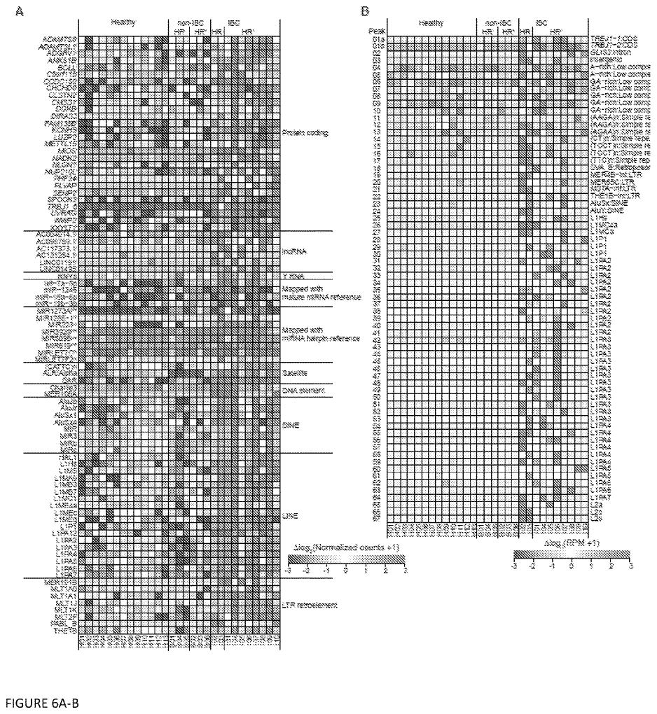

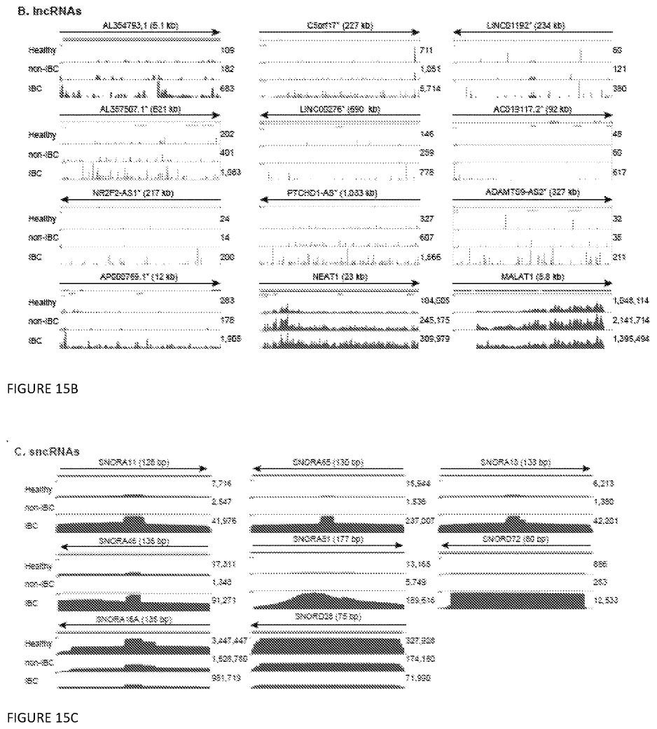

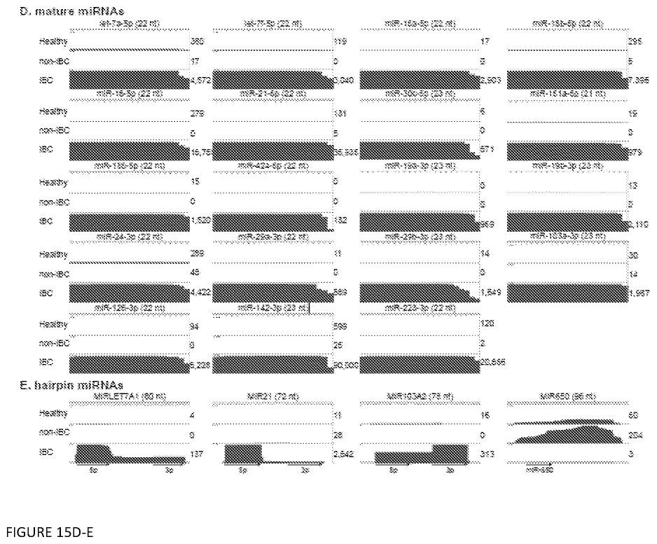

Our analysis of PBMCs identified 577 IBC-vs-non-IBC DE genes (212 protein coding and 365 other) at adj. p≤0.001 and |LFC|≥2 (increasing to 3,380 (1,741 protein coding and 1,639 other) at adj. p≤0.05 and |LFC|≥1). Heat maps, Volcano plots, and IGV plots for notable genes in PBMC samples are shown in FIG. 4, FIG. 14, and FIG. 15, respectively.

The most DE-enriched BROAD hallmark gene set that showed elevated expression in IBC compared to both healthy and non-IBC PBMCs consisted of 144 genes downregulated in response to UV radiation (FIG. 2A, left panel). Among these genes, NEK7 (NIMA related kinase 7), a key player in cell cycle progression required for cytokinesis, had the lowest adj. p-values for the IBC versus non-IBC and healthy comparisons (4.8e-13 and 9.7e-19 relative to non-IBC and healthy, respectively), and SDC2* (Syndecan 2), which encodes a transmembrane (type I) heparin sulfate proteoglycan, a class of molecules involved at multiple steps in the progression of different cancers, had the largest effect size (LFCs 3.3 and 3.6 relative to non-IBC and healthy, respectively; FIG. 4A).

The Broad hallmark TNFα-via-NFκB gene set was the most strongly enriched in upregulated genes in IBC compared to non-IBC PBMCs, yet showed little signs of such enrichment in the IBC-vs-healthy comparison (FIG. 2A). However, several key genes from this gene set were upregulated in IBC PBMC samples compared to both non-IBC and healthy PBMCs (FIG. 4A, FIG. 14A, B). These included NFKB1* itself (adj. p=0.0011/5.4e-8 for IBS vs non-IBC/IBC vs healthy); SERPINB2, a regulator of adaptive immune response (adj. p=0.00018/0.0025); OLR1*, which leads to malignant transformation through activation of inflammation (adj. p=1.4e-6/0.028); NAMPT*, which is overexpressed in PBMCs from patients with adult T-cell leukemia (both adj. p≤0.001); and ATP2B1*, a plasma membrane calcium ATPase involved in the calcium signaling required for granule secretion in granulocytes (both adj. p≤0.001).

Protein-coding gene expression was also used to estimate PBMC cell-type abundances via the CIBERSORTx algorithm (56) (FIG. 5). This analysis indicated elevation of both eosinophils and neutrophils, granulocytic white blood cells that are key players in inflammation (57), in IBC patient PBMCs (MWU p=0.0028 and 0.0010, respectively, for eosinophil and neutrophil abundances in IBC PBMC samples compared to the combined healthy and non-IBC sample sets). There was also significant evidence (p=0.010) for a reduction in resting mast cells, another class of granulocytes, in IBC PBMCs relative to the combined non-IBC patient and healthy donor PBMC sample sets.

Among the genes composing the CIBERSORTx marker panel with expression specificity for eosinophils or neutrophils, 3 exhibited higher expression levels in IBC PBMCs than either non-IBC patient or healthy donor PBMC samples: SAMSN1, most strongly expressed in eosinophils (56), with LFCs 2.4 and 1.5 for IBC versus non-IBC and healthy, respectively, adj. p≤0.001 for both comparisons; VNN3, most strongly expressed in neutrophils (56), with LFCs 2.3 and 1.2 for IBC vs non-IBC and healthy, respectively, adj. p≤0.001 for IBC vs non-IBC and 0.0029 for IBC vs healthy); and MGAM, also most strongly expressed in neutrophils (56), with LFCs 2.1 and 1.6 for IBC vs non-IBC and healthy, respectively, adj. p≤0.001 for IBC vs. healthy, adj. p≤0.01 for IBC vs. non-IBC (FIG. 4A). BRAF, a well-known cancer marker, which is expressed at highest levels in B cells but also at higher levels in eosinophils and neutrophils than other cell types (56), also showed signs of elevated expression in IBC PBMCs (LFCs 1.3 and 1.6 relative to non-IBC and healthy, respectively, adj. p≤0.001 for both comparisons).

As was the case for the FFPE IBC vs non-IBC comparison, we saw little evidence of enrichment the hallmark IL6/JAK/STAT3 signaling gene set among the differentially expressed genes in IBC PBMCs (FIG. 2A). None of the genes in this gene set were differentially expressed at adj. p≤0.001 and LFC≥2, but 3 (MAP3K8, IL1R2, and CXCL1) had adj. p≤0.01 and LFC≥1, and 2 more (IL1R1 and IL1B) had adj. p≤0.05 and LFC≥1. Though not part of the hallmark IL6/JAK/STAT3 signaling gene set, one STAT gene, STAT4, showed an increase (LFC 1.6, adj. p=2.2e-10) in IBC relative to non-IBC samples.

Forty lncRNAs were significantly upregulated in IBC PBMCs compared to healthy donor and non-IBC patient PBMCs (FIG. 4A). Ten of these significantly DE lncRNAs had a previously described relationship to cancer, including AL354793.1, identified as a biomarker for triple-negative breast cancer; LINC01192, associated with low survival of triple-negative breast cancer; and antisense NR2F2-AS1, shown to promote cancer cell proliferation in a number of different cancers. Notably, GCSHP3, a pseudogene that is downregulated in gastric cancer, was expressed at high levels in the two HR-IBC patient samples and at moderate levels in a low borderline HR+IBC patient sample (patient 10), but strongly downregulated in the remaining six HR+IBC patient PBMC samples, mirroring its expression pattern in gastric cancer (FIG. 4A). Two additional lncRNAs NEAT1 and MALATI (NEAT2), which function in DNA repair and play roles in regulating the growth and progression of breast and other cancers, were upregulated in IBC compared to healthy donor PBMC samples (LFC=1.7 and 0.57, adj. p=1.3e-6 and 0.00044, respectively), but less so or not at all in IBC compared to non-IBC patient PBMCs samples (LFC=1.0 and 0.068, adj. p=0.074 and 0.89, respectively; FIG. 4A).

The proportions of cellular and mt tRNA reads were significantly higher in PMBCs from IBC patients than in healthy donors and non-IBC patients (FIG. 1B, top panel; MWU p=1.0e-7 for both cellular and mt tRNAs, IBC vs combined healthy and non-IBC samples), likely reflecting activation of immune and inflammatory responses in IBC PBMCs. This putative activation was accompanied by significant increases in the relative abundance of many tRNA isodecoder families, as well as some tRNA halves and 3′-tRFs fragments, potentially relevant to cell regulatory processes, with the 5′ half of TRE-CTE notable for being significantly downregulated in IBC patient vs both healthy donor and non-IBC patient PBMCs (FIG. 4A Volcano plots FIGS. 14A and B).

Twenty-seven snoRNAs were significantly up-regulated in IBC patient compared to healthy donor and/or non-IBC patient PBMCs (FIG. 4B, FIGS. 14A and B). These included SNORA11 and SNORA65, which were reported to be signatures of ameloblastoma; SNORA13, which has been related to chemoresistance of osteosarcomas; and SNORD72, which is overexpressed in hepatocellular carcinoma. Four snoRNAs were significantly downregulated in both IBC and non-IBC patient PBMCs relative to healthy donor PBMCs (SNORA16A, SNORD28, SNORD41, and SNORD12C; FIG. 4B, FIGS. 14A and B). SNORA105A was notable for being downregulated in IBC compared to both healthy and non-IBC PBMCs (FIG. 4B, FIGS. 14A and B).

Forty mature miRNAs identified by mapping to the miRBase mature miRNA reference set and 22 miRNA transcripts identified by mapping to the miRBase hairpin reference set were significantly upregulated in IBC PBMC samples compared to both healthy and non-IBC PBMC samples (FIG. 4B, FIGS. 14A and B). Significantly upregulated mature miRNAs identified by mapping to the miRBase mature miRNA reference set included miR-21, whose expression levels correlate with the histological grade of IBC (58), and miR-223 and miR-142 both identified as hematopoietic miRNAs capable of increasing the rate at which progenitor cells differentiate into T cells (59) with miR-223 identified as a potential diagnostic marker and therapeutic target for inflammatory disorders (60) (FIG. 4B, FIGS. 14A and B). IGV alignments showed that all the upregulated miRNA transcripts identified by mapping to the hairpin reference also included mature miRNAs that were 1-2 nt shorter or longer than annotated miRNA sequence (denoted with a superscript “m+pre” in that section of FIG. 4B).

Twelve upregulated miRNA transcripts that mapped to the miRBase hairpin reference also contained reads corresponding to longer RNAs, including both full-length pre-miRNAs and RNAs that were likely derived from pre-miRNA but shorter than the annotated hairpin sequence (both indicated by superscript “pre” in FIG. 4B, examples shown in IGV alignments in FIG. 15E). Both the pre-miRNA hairpin and mature form of miR-let-7al and miR-103A2, which promotes proliferation and migration of esophageal squamous cells, were overexpressed in IBC PBMCs compared to both healthy donor and non-IBC PBMCs (FIG. 4B). miR-650 pre-miRNA hairpin fragments were significantly elevated in both healthy and non-IBC compared to IBC PBMCs (FIG. 4B and FIG. 15E).

Finally, 16 Repeat-element RNAs were significantly upregulated in IBC compared to healthy and non-IBC PBMC, including 12 simple repeats or satellite sequences, 3 LTR elements (LTR89, MLT1-int, and PRIMA41-int) and one LINE-1 element L1PB (FIG. 4B, FIGS. 14A and B).

### Identification of Potential IBC Biomarkers in Plasma

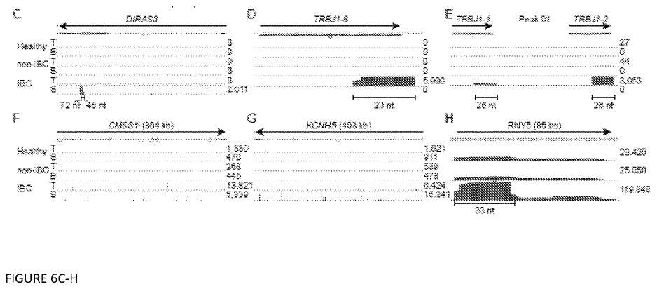

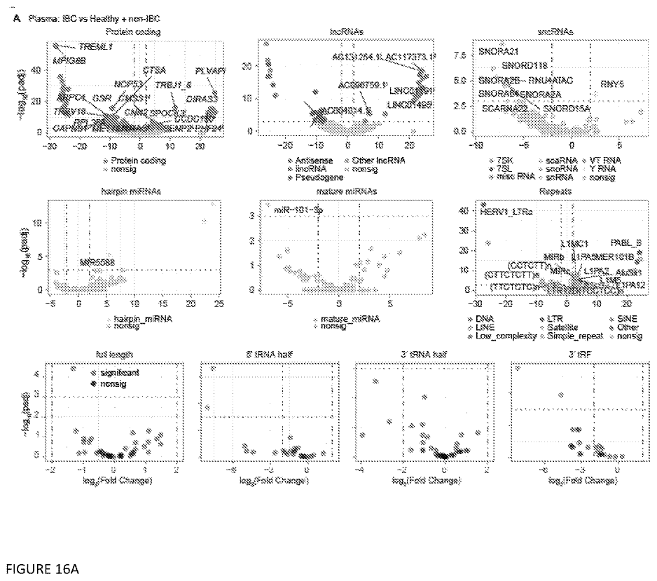

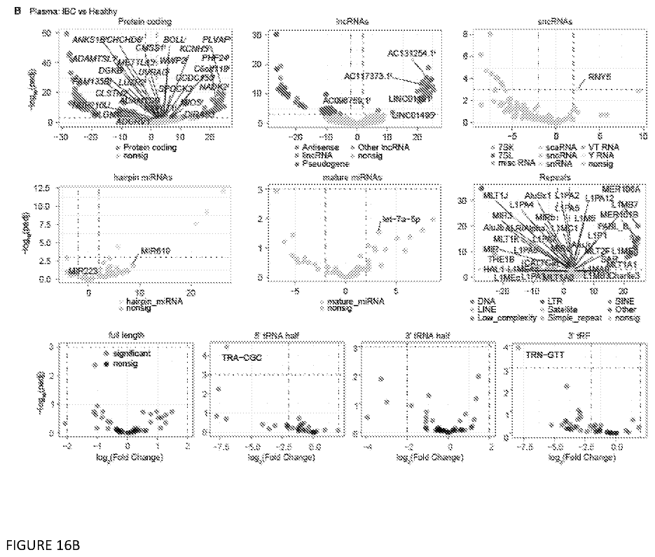

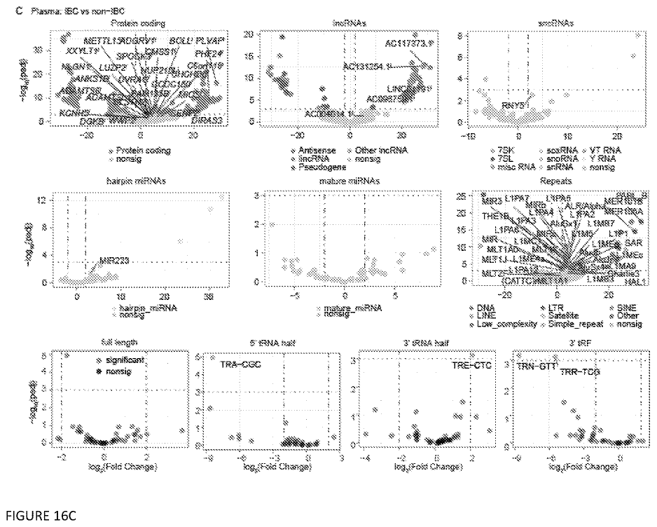

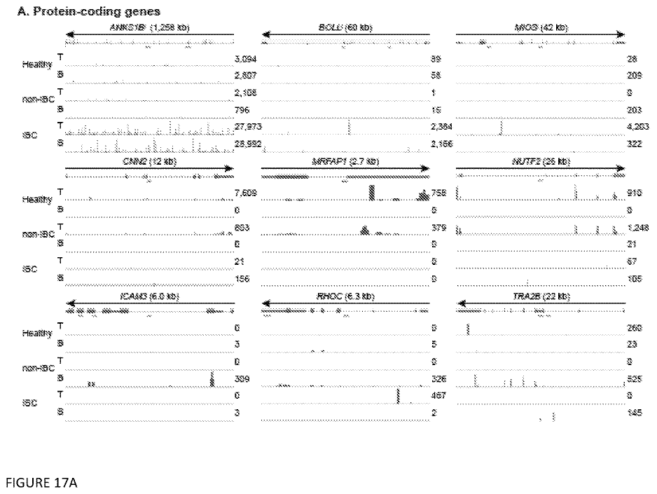

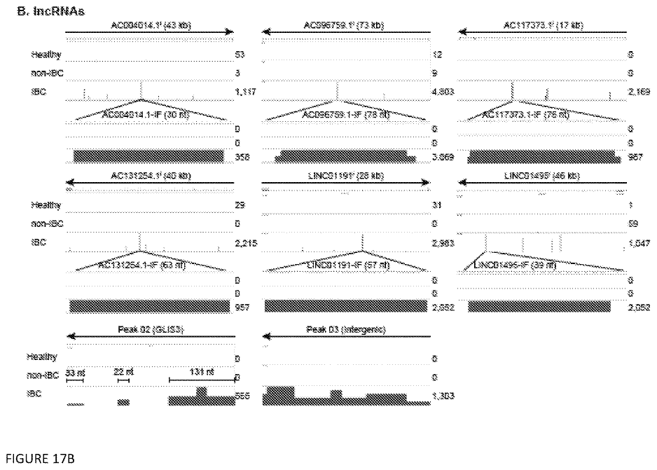

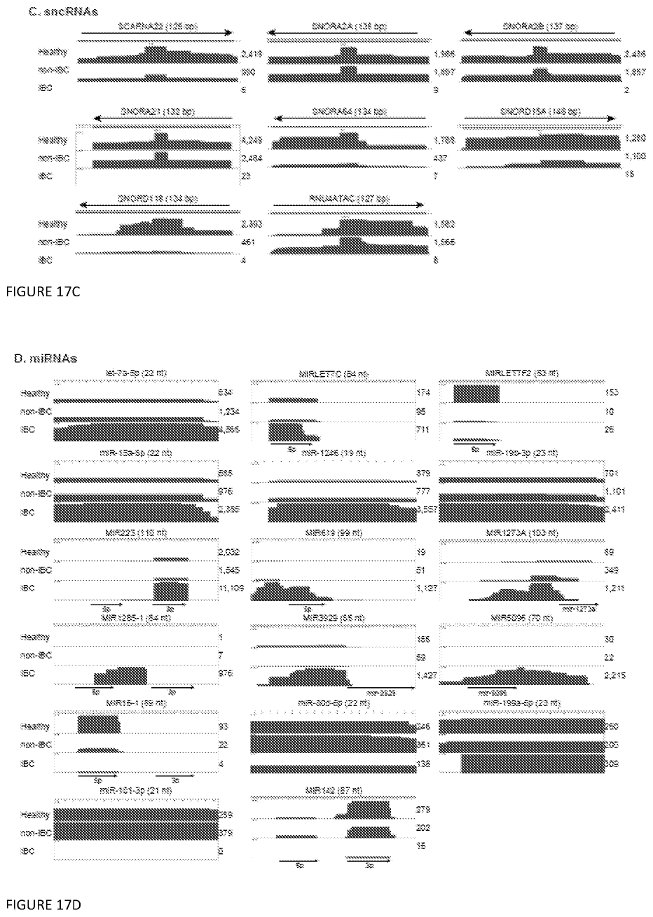

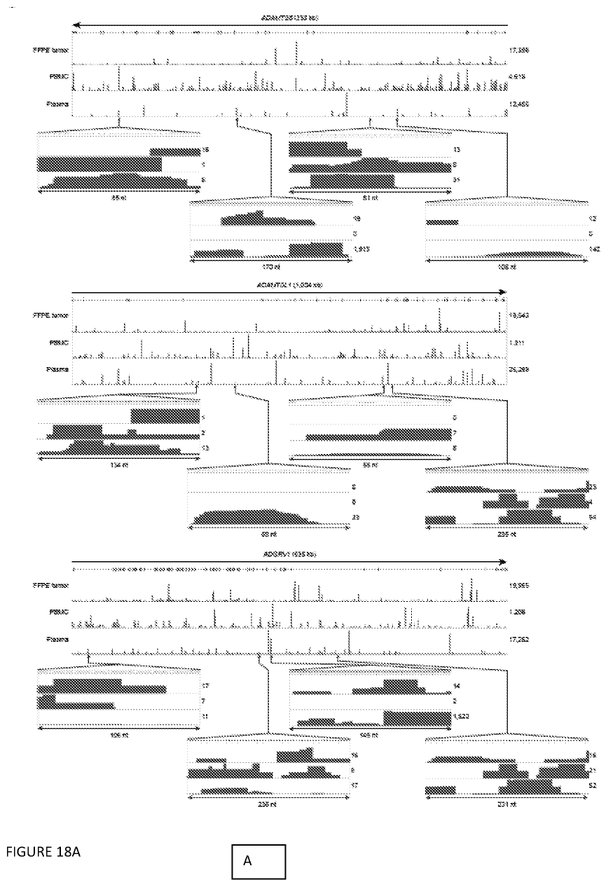

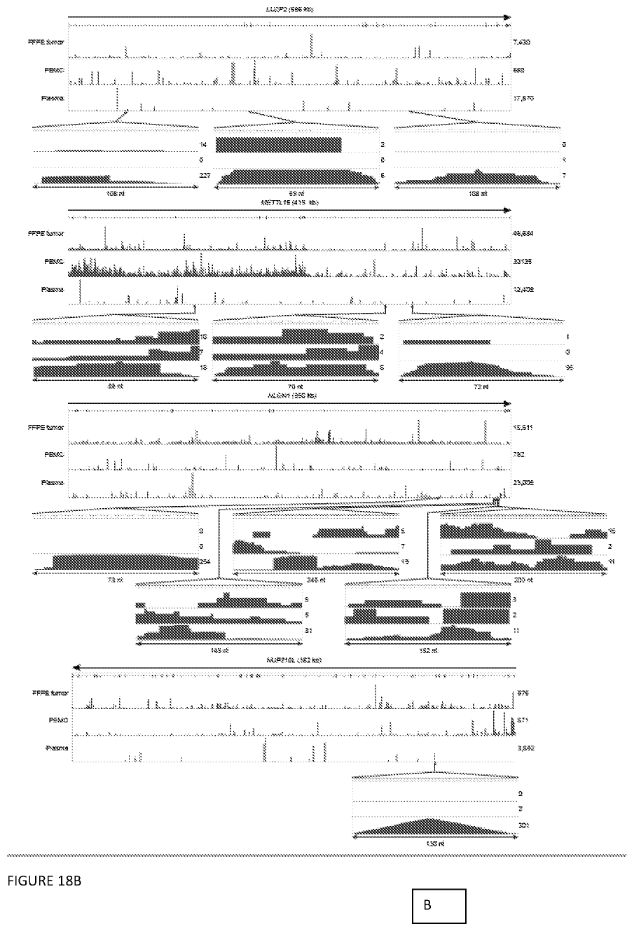

Previous TGIRT-seq analyses found that human plasma from healthy individuals contains a complex mixture of largely fragmented mRNAs and lncRNAs, likely reflecting digestion of accessible non-protein bound regions by plasma RNase, together with full-length mature tRNAs and other structured sncRNAs, mature miRNAs, pre-miRNAs, other transcripts of miRNA loci, and a variety of Repeat-element RNAs (2, 3). TGIRT-seq of the clinical plasma samples in this study gave datasets with RNA sequences mapping to up to 22,000 different genes encompassing all of the above categories (Supplemental file). In addition to significantly over-represented RNAs identified by DESeq2, we also used TGIRT-seq peak calling (3) to detect significantly over-represented RNA fragments (read coverage ≥10 reads at the peak maximum, q-value≤0.01). Significantly over-represented RNAs identified by DESeq2 and detected in at least half of the IBC samples included 30 protein-coding gene RNAs, 6 lncRNAs, 1 sncRNA (RNY5), 39 Repeat-element RNAs, and those identified by peak calling and also detected in at least half of the IBC samples included 2 protein-coding gene RNAs, 2 lncRNAs (one intronic), and 64 Repeat-element RNAs (heat maps FIG. 6, Volcano plots FIG. 16, IGV alignments FIG. 17). The number of significantly over-represented RNAs identified in IBC plasma by DESeq2-in all comparisons, IBC vs non-IBC, IBC vs healthy, and IBC vs combined non-IBC and healthy sample sets-increased from 76 to 546 for adj. p≤0.05 and LFC≥1. A number of mature miRNAs (let-7a-5p, let-7c-5p, let-7f2-5p, miR-15a-5p, miR-1246-5p, miR-19b-3p and miR-223-3p) and partially or aberrantly processed miRNA transcripts (mir-619, mir-1273a, mir-1285-1, mir-3929, and mir-5096) detected in 5 or more IBC samples were over-represented (LFC≥2) in IBC compared to healthy and non-IBC samples but fell short of statistical significance, likely due to low read counts and variable quality of the plasma samples (FIG. 6A).

Protein-coding gene RNAs that were significantly over-represented in IBC plasma included 4 corresponding to short, discrete, largely exonic RNA fragments, possibly stabilized in plasma by bound proteins (FIG. 6A). For one gene, DIRAS3, a tumor suppressor gene whose normally silenced maternal allele can be re-activated to induce autophagy in breast cancer cells, the over-represented species were 72- and 45-nt RNA fragments derived from the 3′ end of the coding sequence and 3′ UTR, respectively (FIGS. 6A and C). The other 3 over-represented exon-containing RNAs were 23- or 26-nt RNA fragments of 3 different T-cell receptor beta joining region one segments (TRBJ1-1, -2, and -6; FIGS. 6A, B, D, and E). The TRBJ1-6 RNA was detected in all 10 of the IBC samples (highly abundant in 9, moderately abundant in 1 and not detected in any of the healthy or non-IBC samples) (FIG. 6A). The TRBJ1-1 RNA was abundant in 4 of the IBC samples and not detected in any of the healthy or non-IBC samples, while the TRBJ1-2 RNA was found in all 10 of the IBC samples (highly abundant in 7 and moderately abundant in 3) and in lower abundance in one each of the healthy and non-IBC samples (FIG. 6B).

Surprisingly, the remaining 26 protein-coding gene RNAs that were significantly and consistently over-represented in IBC patient plasma corresponded to 50- to 80-nt intron RNA fragments (denoted with superscript “i” in FIG. 6A; IGV examples in FIG. 6F, G and FIG. 17A, top line). Intron RNA fragments were also the overrepresented RNA species for all 6 lncRNA genes that were significantly over-represented in IBC plasma (FIG. 6A, IGV plots in FIG. 17B). Some of the over-represented intron RNAs were highly specific for IBC plasma (e.g., PHF24 and PLVAP, AC117373.1), while others were sparsely present in healthy and/or non-IBC plasma (FIG. 6A).

For RNY5, the over-represented RNAs in IBC plasma were 5′ fragments of 28-33 nt (FIG. 6H), similar to those that were found to be enriched in cancer cell extracellular vesicles and cause rapid death of human primary cells but not cancer cells, suggesting a mechanism by which cancer cells might establish a favorable environment for proliferation (61). No tRNAs, RNA halves or 3′ tRFs were found to be significantly over-represented in IBC plasma at adj. p≤0.001 and |LFC|≥2 compared to non-IBC and healthy plasma (FIG. 16A-C). A 165-nt RNA identified as over-represented in IBC plasma by peak calling mapped to an intergenic region of chromosome 7 (denoted “Intergenic” in FIG. 6B).

The 39 Repeat-element RNAs identified as significantly over-represented in IBC plasma samples by DESeq2 included 18 LINEs, 8 SINEs, 8 LTR-containing retroelements, 2 DNA transposons, and 3 satellite sequences (FIG. 6A), while the 64 Repeat-element RNA fragments identified by peak calling in IBC ranged from 151 to 478 nt and included 43 mapping to LINEs, 5 to LTR-containing retroelements, 2 to SINEs, and the remainder to low complexity sequences, which include simple repeats and satellite sequences (FIG. 6B). Notably, all the LINE-1 RNAs identified as over-represented in IBC plasma by DESeq2 corresponded to the LINE-1 sense strand, while all those identified as over-represented by peak calling corresponded to the LINE-1 antisense strand.

Reciprocally, we identified numerous genes whose transcripts were significantly over-represented and present in at least half of the healthy and non-IBC plasma samples, but sparsely represented in IBC plasma samples (heat maps FIGS. 7A and B, Volcano plots FIG. 16A-C, IGV examples in FIG. 7D and FIG. 17). These included 125 protein-coding gene RNAs, 1 lncRNA (SNHG6 suggested to promote cancer development by miRNA sponging), 8 sncRNAs (7 snoRNA and snRNA U4ATAC), the 5′ half of AlaCGC and 3′-tRFs of AsnGTT and ArgTCG, 5 mature miRNAs (mir-16-1-5p, mir-30d-5p, mir-199a-5p, mir-101-3p, and mir-142-3p), 4 simple repeats (CCTCTTn, TCCTCCn, TTCTCTCn, and CTTCTCTTn), and 2 LTR retroelement RNAs (HERV1-LTRc and LTR12D) (FIGS. 7A and B). Three other protein-coding gene RNAs and 1 lncRNA were found to be over-represented in non-IBC plasma compared to both healthy and IBC plasma (FIG. 7C).

In contrast to IBC plasma, where the over-represented RNAs for protein-coding genes were largely intronic, with 3 exceptions (PTK2B, SNCA, and ZMPSTE24), the over-represented RNAs for protein-coding genes in healthy and non-IBC plasma mapped largely to exons, indicating that they were fragmented mRNAs (examples in FIGS. 7A and D and FIG. 17A, bottom two lines). This stark difference between IBC and non-IBC plasma samples is illustrated by the finding that although introns comprised substantial proportions of the bases in reads mapping to protein-coding genes in all plasma samples regardless of disease state (healthy, 65%; non-IBC, 40%; and IBC, 87%), they comprised >91% of the bases in reads from over-represented protein-coding genes in IBC plasma compared to only 10 and 6% for over-represented protein-coding genes in healthy and non-IBC plasma samples, respectively (FIG. 7E).

### Origin of TRBJ1 RNAs in IBC Patient Plasma

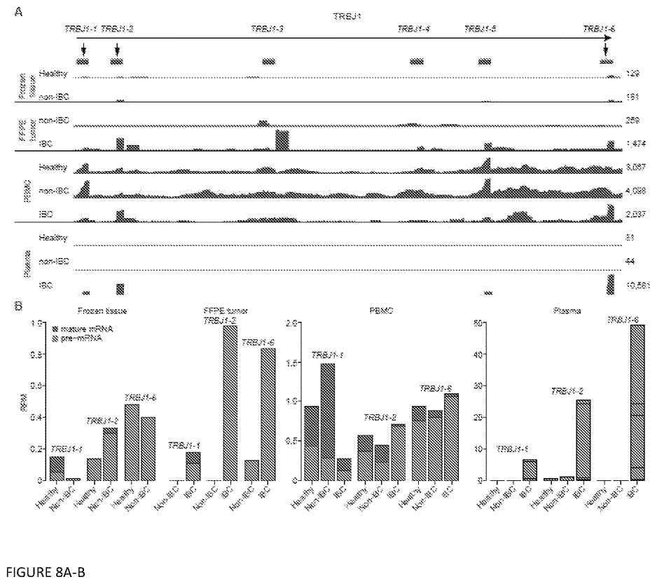

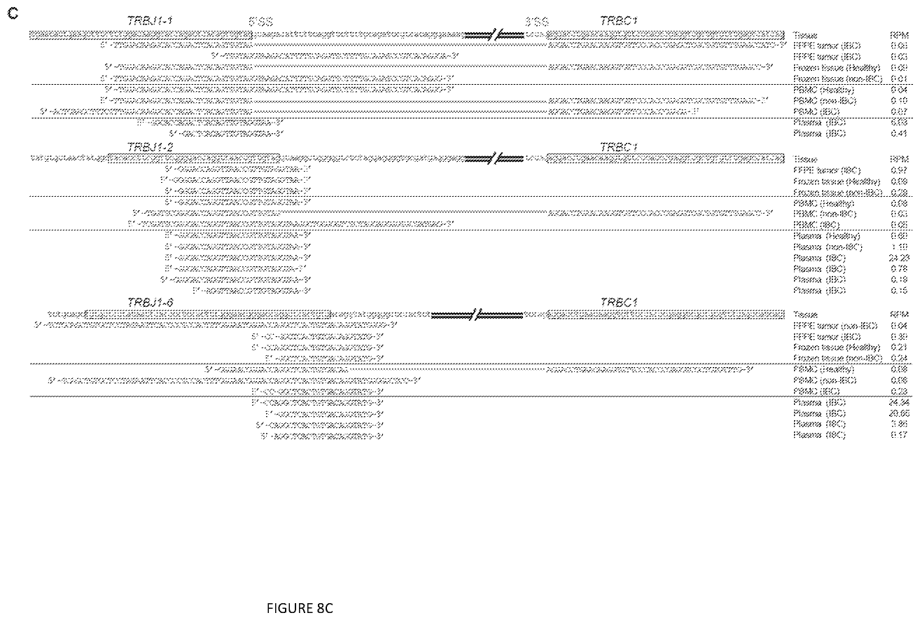

The cellular origin of the 3 TRBJ1 RNA fragments identified above as potential plasma RNA biomarkers for IBC was revealed by IGV alignments (FIG. 8A), which showed peaks corresponding to the same TRBJ1-2 and -6 and to a lesser extent TRBJ1-1 and -5 RNAs rising above a background of reads spanning these regions in IBC tumor and PBMCs. Despite the presence of RNAs mapping across the TRBJ1 locus in the cellular samples, the reads detected in plasma were short with relatively discrete lengths, suggesting they corresponded to RNA fragments protected from plasma RNase by bound proteins (3).

Closer scrutiny revealed that all of the exonic TRBJ1 reads from plasma samples, regardless of disease state, extended a short distance into a downstream intron, while those in the cellular samples were a mixture of exon-intron and spliced mRNA reads in which the different TRBJ1 segments were spliced to the downstream TRBC1 constant region (FIGS. 8B and C). The spliced TRBJ1-TRBC1 mRNA read are expected for TRBJ1-n segments that encode productively rearranged TCRb VDJ in T-cells. Among the cellular RNA samples, the preponderance of TRBJ1-2 and -6 exon-intron reads was highest in IBC FFPE tumors (FIG. 8B), where the TRBJ1 reads may have originated either from unspliced pre-mRNA in tumor infiltrating T cells or from aberrant transcription of the TRBJ1-n locus in non-T-cells, possibly reflecting wide-spread enhanced transcription in IBC tumors (FIGS. 8B and C). Regardless of their origin, the TRBJ1-2 and TRBRJ1-6 RNAs, which were prevalent in IBC patient plasma samples but sparse or undetectable in non-IBC patient or healthy donor plasma samples, are strong candidates for plasma RNA biomarkers for IBC.

### Origin of Intron RNAs Fragments in IBC Patient Plasma

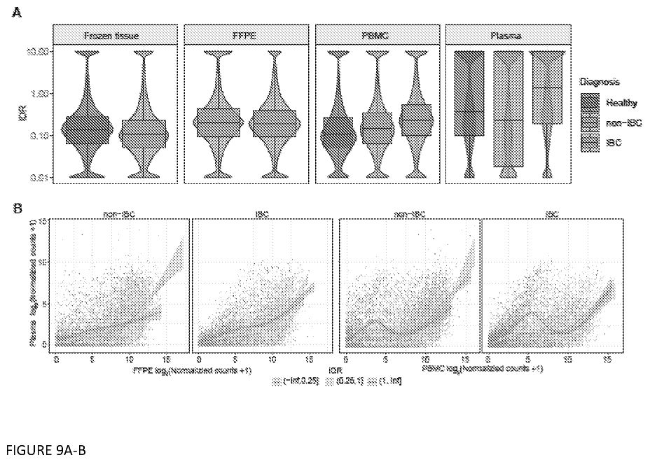

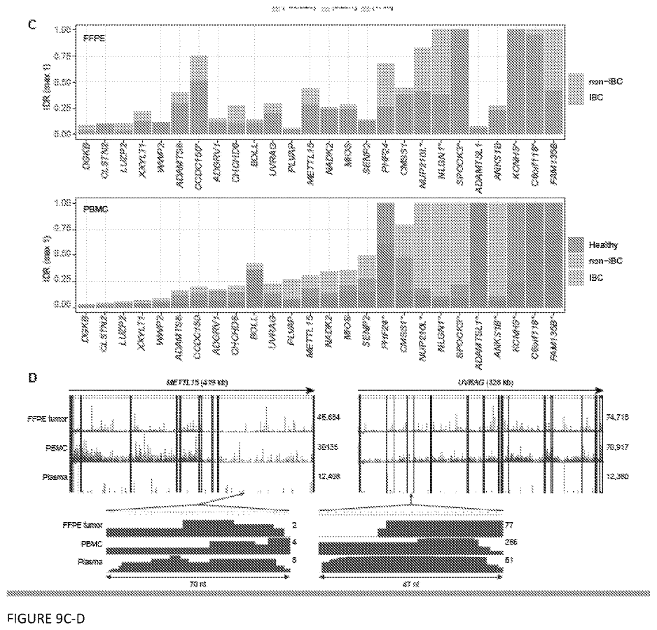

We wondered whether the prevalence of significantly over-represented intron RNAs in IBC plasma samples might be related to the accumulation of unspliced pre-mRNAs and/or excised intron RNAs from genes with high IDRs (FIGS. 6 and 7 and Table 2). To investigate this further, we compared IDRs for protein-coding genes between different sample types and found overall higher IDRs in PBMCs from IBC patients compared to those from non-IBC patients or healthy donors with this difference magnified in the IBC plasma samples (FIG. 9A). Although the average abundances of individual protein-coding gene RNAs in both non-IBC and IBC plasma samples correlated with those in FFPE tumor and PBMC samples across all genes with different IDRs (FIG. 9B, different colored trend lines and dots), this correlation was higher for genes with high IDRs (≥1) in FFPE tumors and particularly in PBMCs (FIG. 9B, red dots and trend lines). The elevation of average plasma RNA levels for higher-IDR genes was most pronounced for those genes with relatively low expression levels in FFPE tumor or PBMC samples (FIG. 9B, x-axis log 2-expression coordinates in the 2.5-7.5 range), with RNAs from a clear subset of high IDR genes within this range of expression levels present at much higher levels in plasma than RNAs from lower IDR genes (FIG. 9B, red points above trend lines at left side of each panel). To quantify these phenomena, we calculated the Spearman correlations (rs) between IDR and the difference in plasma vs FFPE tumor or PBMC RNA levels across all genes. We found that this correlation was significantly positive (Spearman correlation test p-value<1e-10) for all four comparisons in the four panels of FIG. 9B, but with higher correlations for IBC tumors and PBMCs (rₛ=0.12 and 0.15, respectively) than for non-IBC FFPE tumors and PBMCs (rₛ=0.05 for both). These findings suggest that both IDR and disease-specific differences contribute to the prevalence of intron RNA fragments in IBC plasma.

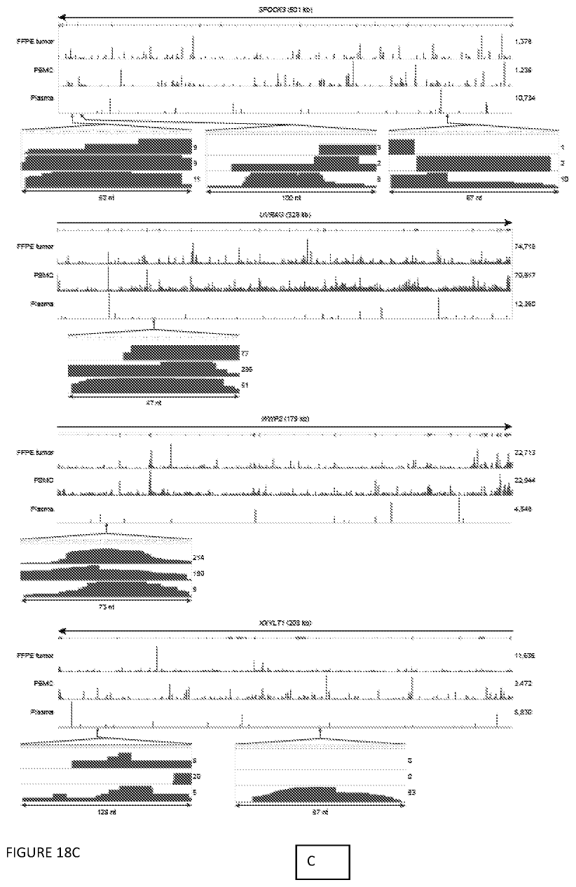

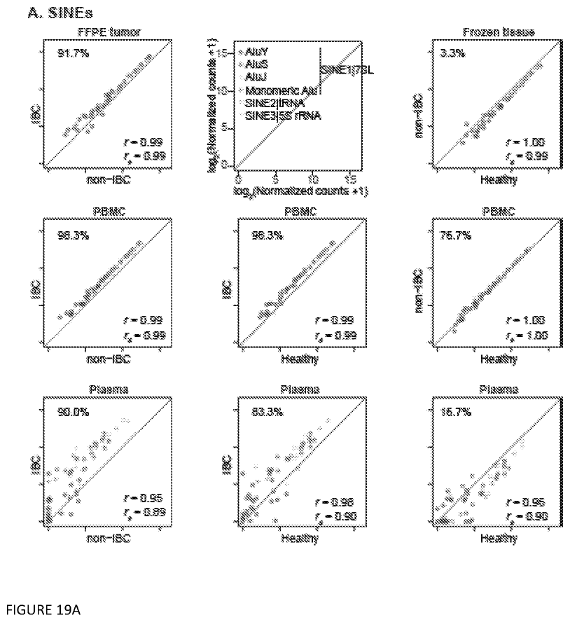

A substantial proportion of the 26 protein-coding genes identified by DESeq2 as having over-represented intron RNA fragments in IBC plasma (FIG. 6A) had higher and in some cases dramatically higher IDR values in IBC tumor and/or PBMC samples than in non-IBC or healthy donor samples (FIG. 9C). For these 26 genes we identified a total of 2,481 intron fragments (where overlapping fragments were considered one fragment), including 40 intron RNA fragments that were over-represented in IBC plasma and found in at least half of the IBC plasma samples, making them candidates for IBC biomarkers (2 examples showed in FIG. 9D; others in FIG. 18). Collectively, these finding indicate that many of the intron RNAs found in IBC plasma samples were derived from high IDR genes in IBC tumors and/or PBMCs, coupled with preferential export and/or stabilization of the intron RNA fragments in IBC plasma.

### Activation of LINE and Other Retroelements Elements in IBC

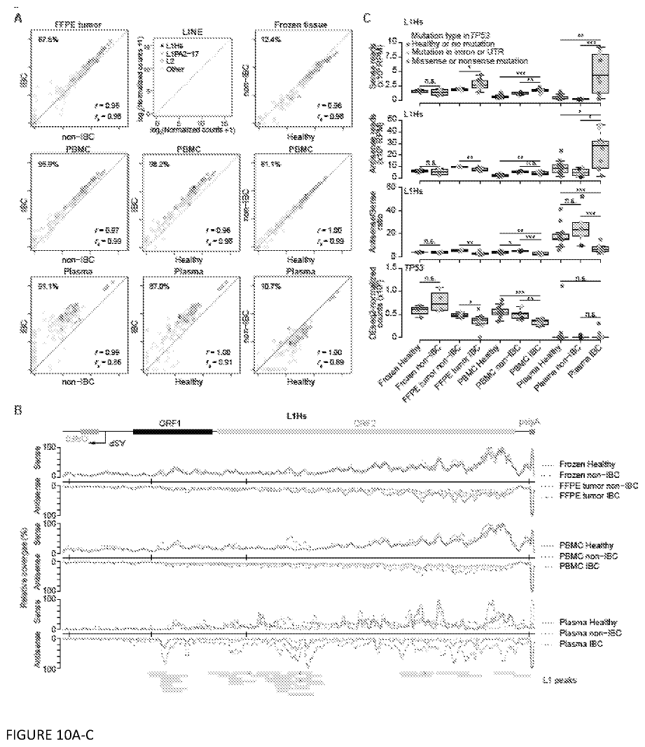

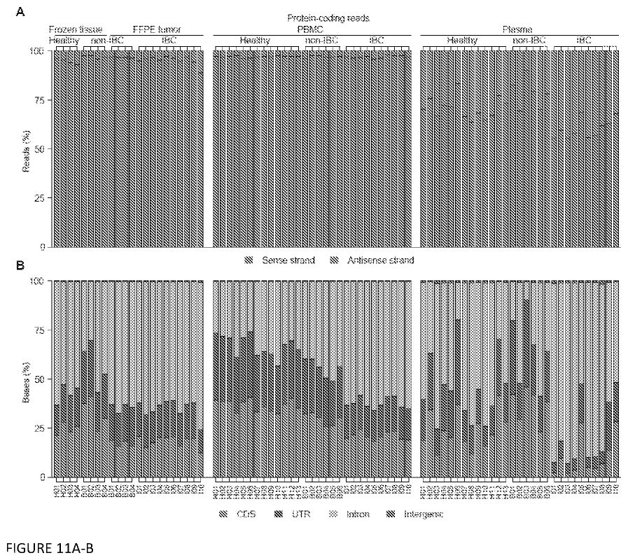

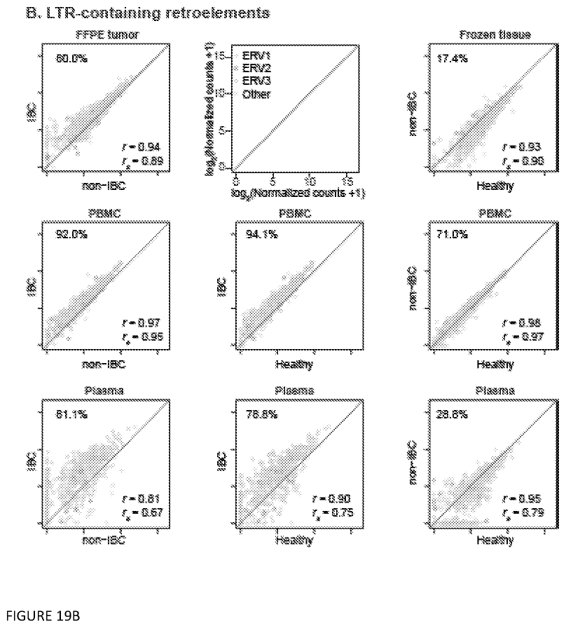

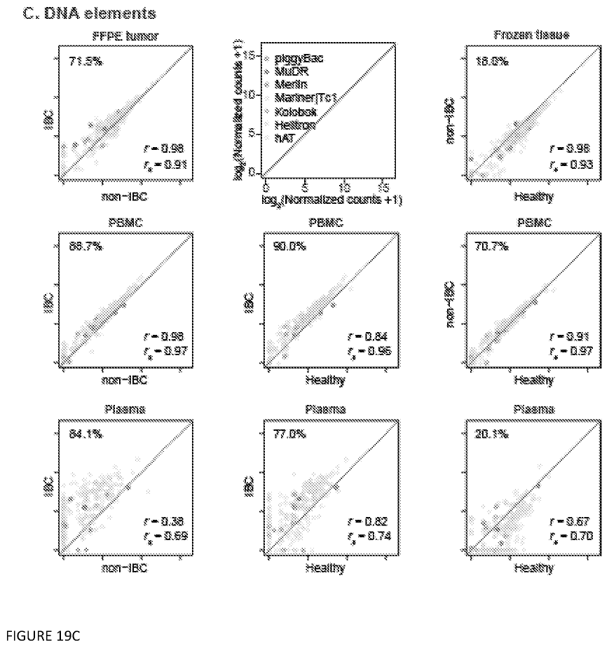

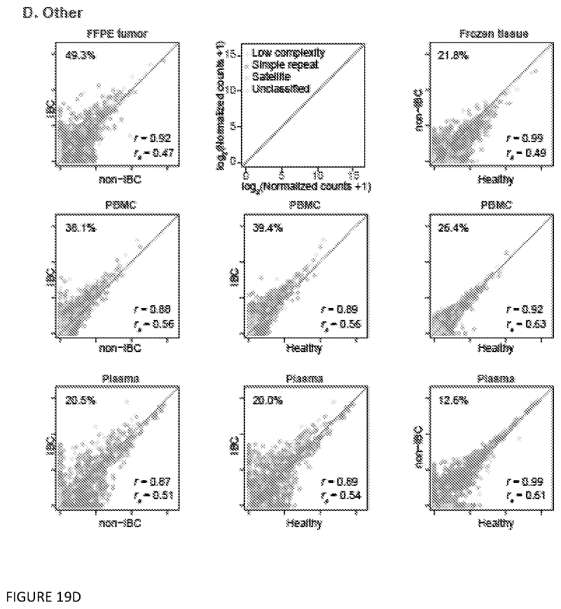

Finally, findings above identified LINE and other Repeat-element RNA fragments as being among the most prevalent differentially represented RNAs in IBC plasma samples compared to those from non-IBC patients or healthy donors (FIG. 6). To investigate if this might reflect global activation of Repeat element families in IBC, we aggregated reads mapping to multiple loci of each Repeat-element family and used scatterplots to compare the aggregated counts for those families in cellular RNAs. The scatterplots indicated overexpression of multiple Repeat-element families in IBC compared to healthy and non-IBC breast tissue and PBMC samples, including known active human LINE-1s (L1Hs), as well as and primate-specific LINE-1s (L1PA2-17), LINE-2s, SINEs, endogenous retroviruses and other LTR-containing retroelements, and to a lesser extent DNA transposons and other Repeat-element RNAs (FIG. 10A and FIG. 19). The higher abundance of LINE and other Repeat-element RNAs in IBC tumors and PBMCs was magnified in plasma, suggesting preferential enrichment of these RNAs in plasma relative to other RNA biotypes (FIG. 10A and FIG. 19). By contrast, pairwise comparisons of non-IBC and healthy control samples showed no similar enrichment of LINE and other repeat elements RNAs in non-IBC tumors, PBMCs, or plasma (FIG. 10A and FIG. 19).

For further analysis, we analyzed reads mapping to L1Hs, an active human-specific LINE-1 element (62). Such reads, which may include those from other LINE-1 elements with similar sequences, were distributed throughout the length of L1Hs with somewhat higher density near the 3′ end of L1Hs RNA, possibly reflecting contributions from truncated LINEs, and included both sense and antisense strand LINE RNA reads, with the latter predominating, particularly in plasma (FIGS. 10B and C). Full-length LINE-1 antisense RNAs could result from activation of an as yet undetected LINE-1 promoter, but more likely from read through transcription from downstream genes or transcription of genes in which LINE-1s had inserted within introns in the antisense orientation. The ratios of LINE-1 elements inserted in the antisense and sense orientations in human cell lines was found to 1.13 and 1.8 in different studies (63, 64). Notably, the ratios of antisense to sense strand reads mapping to L1Hs in plasma samples were substantially higher than these values or the ratios of antisense to sense strand reads mapping to L1Hs in the cellular RNA samples (FIG. 10C), indicating preferential enrichment of LINE-1 antisense RNAs in plasma.

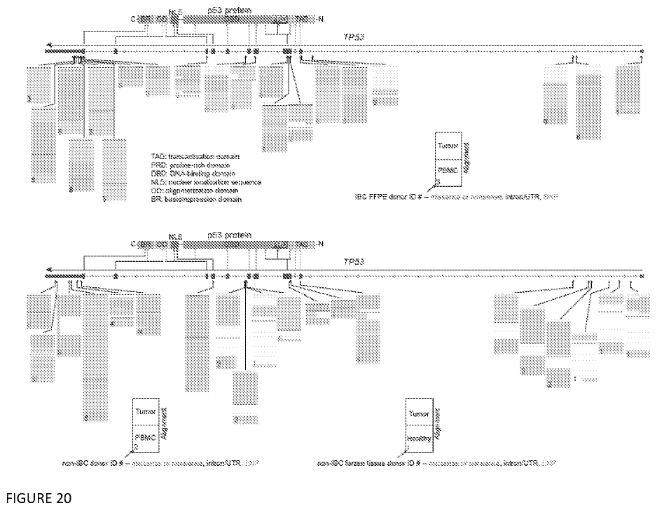

To explore the origin of L1Hs sense and antisense strand reads, we examined the status of the TP53 gene, a known LINE-1 repressor in the IBC patient samples, and found that the abundance of L1Hs sense-strand reads in IBC tumors and PBMCs (FIG. 10C, top panel) was correlated with putative TP53 somatic mutations and/or lower levels of TP53 RNA in IBC tumors (FIG. 10C bottom panel; see FIG. 20 for mapping of putative TP53 mutations) (65, 66). By contrast, the abundance of L1Hs antisense reads in IBC tumors and PBMCs did not show a similar correlation, suggesting that they arose via transcription of host or downstream genes in IBC, potentially enhancing their utility as blood-based biomarkers (FIG. 10C). In non-IBC tumors and PBMCs, the levels of L1Hs sense-strand reads remained equal to or lower than those in the corresponding frozen healthy breast tissue or FFPE IBC tumor samples, despite the presence of putative TP53 somatic mutations in non-IBC tumor samples (FIG. 10C), likely reflecting diverse, layered mechanisms that control the overall abundance of LINE-1 RNAs in human cells (62, 67). The presence of both sense and antisense LINE-1 reads in IBC samples is consistent with the presence of LINE-1 dsRNAs, which have been suggested to induce an inflammatory response in multiple human cell types via RIG-I and MDA5 sensing and activation of interferon signaling (68), potentially highly relevant to IBC.

### Discussion

Here, we used TGIRT-seq to profile protein-coding and non-coding RNAs in tumor slices, PBMCs and plasma from IBC patients compared to non-IBC patients and healthy donors, to our knowledge the first such comprehensive RNA analysis for any cancer or disease. We identified hundreds of significantly DE genes at adj. p≤0.001 and |LFC|≥2 in IBC tumors and PBMCs, increasing to thousands with adj. p≤0.05 and |LFC|≥1. The DE protein-coding genes identified as having significantly elevated RNA expression levels in IBC tumors included key members of the BROAD TNFα-via-NFκB and EMT gene sets, which were also found to be DE in IBC in previous studies (12-14, 27). By contrast, except for JUN, an inflammatory response modulator that is also a member of the TNFα-via-NFκB and EMT gene sets (49), we found little evidence for elevated RNA expression of genes of the Broad IL6/JAK/STAT pathway, which appears to be activated largely at the protein level in IBC tumors and PBMCs (17, 43).

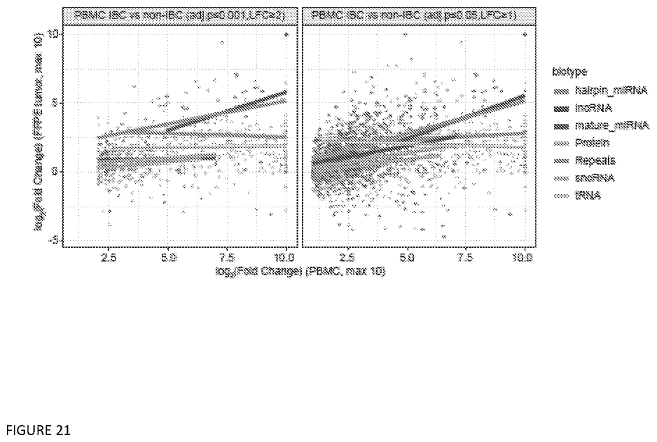

Overall, we found that PBMCs from IBC patients were more highly activated than those from non-IBC patients or most healthy donors, as judged by elevated levels of cellular MtRNA, which could support increased rates of protein synthesis, as well as elevated levels of snRNAs, which could support increased rates of RNA splicing (FIG. 1B). Major pathways identified as being activated in IBC PBMCs were Broad TNFα-via-NFκB and UV response down, including ATP2B1*, a member of both pathways involved in granule secretion in granuloctyes (Supplemental file). The CIBERSORTx algorithm, which estimates the abundances of different cell types from gene expression data, indicated elevated abundance of granulocytic eosinophils and neutrophils in PBMCs from IBC patients compared to those from non-IBC patients and healthy donors. In addition to being associated with innate immune responses to parasitic infections, eosinophils are also known to play a role in an anti-tumor response (69). Despite the likely presence of tumor infiltrating PBMCs, we found only limited overall correlation of LFCs for significantly DE genes encoding different RNA biotypes between IBC PBMCs and tumors (FIG. 21).

Taking advantage of the ability of TGIRT-seq to quantitate levels of intron RNAs, we developed a bioinformatic parameter termed intron-to-exon depth ratio (IDR), which enabled us to distinguish protein-coding genes with two different expression patterns, one with lower IDRs corresponding to mRNAs with reads mapping largely to exons, and the other with higher IDRs reflecting higher levels of reads arising from different introns of the gene (FIG. 1B). Approximately 18% and 62% of significantly overexpressed protein-coding genes (adj. p≤0.001 and |LFC|≥2) in IBC tumor and PBMC samples, respectively, had IDRs ≥0.5 in IBC, while no such significantly overexpressed protein-coding gene was found in non-IBC tumors or PBMCs, with similar patterns holding for much larger numbers of DE genes with lower significance and fold-change cutoffs adj. p≤0.05 and |LFC|≥1; (Table 2). The higher percentage of DE high IDR genes in IBC suggests an imbalance between the rates of transcription and RNA splicing and/or intron RNA turnover. In general, this imbalance could reflect higher rates of transcription and/or lower rates of RNA splicing or intron RNA turnover, all of which could vary in different ways for each gene or intron. In IBC tumors, this imbalance might reflect activation of genes involved in oncogenesis and cellular counter measures. In IBC PBMC samples, this imbalance likely reflects activation of immune responses resulting in higher rates of transcription of overexpressed genes, so that RNA splicing and/or intron RNA turnover become rate limiting.

The wide-spread imbalance in the rates of transcription and RNA splicing in IBC tumors and PBMCs may be related to hypertranscription, a phenomenon recently reported for a number of aggressive cancers (47, 48). If so, TGIRT-seq might provide a general method for identifying different degrees of hypertranscription and distinguishing it from more restricted elevated transcription of specific gene sets in cancers. Although further studies will be needed to sort this out, a practical consequence of IDR analysis was the identification of numerous protein-coding and lncRNA genes with high IDRs in IBC and the identification of unspliced pre-mRNAs and intron RNA fragments from these genes as potential biomarkers in IBC plasma samples. RNA-seq methods that focus on poly (A)-containing mRNAs would not only miss such features but might also identify genes with high IDRs as under-expressed due to lower levels of spliced mRNAs.

Consistent with previous TGIRT-seq analysis of human plasma (2, 3), we found that intron RNAs contributed substantial proportion of protein-coding gene bases in plasma than in cellular RNA samples irrespective of disease state (FIG. 7E). Surprisingly, however, while most of the abundant differentially represented protein-coding gene RNAs in plasma samples from IBC patients were pre-mRNA or intron RNA fragments, most of those in plasma samples from healthy and non-IBC patients were fragmented mRNAs with reads mapping to multiple exons (FIG. 7E, examples shown in FIGS. 6F and G and FIG. 7D). Further analysis revealed that this difference was related not only to the higher number of differentially expressed genes with high IDRs in IBC tumors and PBMCs, but also to disease-specific differences that favor accumulation of unspliced pre-mRNA and intron RNA fragments in IBC plasma (FIG. 9). The latter could reflect preferential breakage of diseased cells, closer proximity to blood or lymph for dispersed IBC tumors, or wide-spread enhanced transcription that saturates the splicing apparatus and triggers mechanisms for cellular export of intron RNAs in IBC. Importantly, a significant proportion of intron RNAs identified in IBC plasma samples corresponded to intron RNAs that were present in higher abundance in IBC tumors and/or PBMCs, supporting their utility as blood-based markers with direct links to disease progression (FIG. 9). It will be of interest to investigate whether higher levels of intron RNAs and their preferential enrichment in plasma are also characteristic of other aggressive cancers.
1. Wide-spread enhanced transcription due to DNA hypomethylation and changes in chromatin structure likely also contribute to overexpression of LINE and other retroelements in IBC tumors and PBMCs and the enrichment of these RNAs in IBC patient plasma (FIGS. 2B, 6A, and 10). LINE and other retroelements are activated in many cancers (70, 71), but to our knowledge such activation has not been reported for PBMCs. The presence of both sense and antisense reads that extend across the length of the active hominoid LINE-1 (L1Hs) in IBC tumors and PBMCs is consistent with findings suggesting that LINE-1 elements can form dsRNAs that induce an inflammatory response in human cell types via RIG-I and MDA5 sensing and activation of interferon signaling (68), potentially highly relevant to IBC. The enrichment of LINE RNAs in plasma led to the identification of multiple sense and antisense LINE RNAs as potential plasma biomarkers for IBC (FIG. 6). Antisense LINE RNAs, whose over-expression in IBC may reflect enhanced transcription of downstream genes or host genes with LINEs inserted within introns in the antisense orientation, have the potential to be particularly useful as biomarkers, provided they have a unique identifiable sequence that can be mapped to a specific genomic locus.

A major goal of this study was to identify potential plasma RNA biomarkers for IBC that could be used for noninvasive liquid biopsies to differentiate IBC from non-IBC as well as monitor diseases progression and response to treatment. Among the many DE RNAs in IBC, prime candidates for such blood-based IBC biomarkers included 3 different TRBJ1 exon-intron RNA fragments, possibly related to aberrant enhanced transcription and/or induction of a specific immune response in IBC. These 3 TRBJ1 RNAs could be traced to tumors and PBMCs in IBC patients, respectively, supporting their utility as blood-based RNA biomarkers with direct links to disease progression. The TRBJ1-6 pre-mRNA fragments, which we detected in all 10 IBC plasma samples but none of the healthy or non-IBC plasma samples, were likewise not detected by TGIRT-seq of plasma prepared from a healthy individual at different times (PRJNA279866), from different lots of commercial plasma pooled from multiple healthy individuals (PRJNA640428), nor from COVID-19 patient PBMCs or plasma (15 and 20 patients, respectively; unpublished data), advancing them as a prime candidate for an IBC-specific plasma RNA biomarker.

Our study has the following limitations. Although TGIRT-seq identified numerous potential RNA biomarkers for IBC in tumor, PBMC, and plasma samples, validating the clinical utility of these biomarkers for diagnostic, predictive, and/or prognostic purposes will require high-throughput targeted assays of larger numbers of samples. Our study found that intron and retroelement RNAs are enriched in plasma, but mechanisms by which this occurs require further investigation. Finally, although we identified somatic TP53 mutations and/or decreased TP53 RNA levels in IBC tumors as factors that contribute to activation of LINE elements in IBC, other factors that modulate this activation, suppress LINE activation despite TP53 mutations in non-IBC tumors, and activate LINE elements in IBC PBMCs require further investigation.

### Materials and Methods

### Patients and Samples

Ten IBC patient FFPE tumor tissue and paired PBMC and plasma samples, 4 non-IBC patient FFPE tumor tissue and paired PBMC and plasma samples, 2 additional non-IBC patient

PBMC and plasma samples, and 13 healthy donor PBMC and plasma samples were obtained from the Morgan Welch Inflammatory Breast Cancer Research Program and Clinic at MD Anderson Cancer Center (MDACC). Each patient and healthy donor gave written informed consent. This study was approved by the MDACC Institutional Review Board. IBC was diagnosed by strictly following the criteria of the American Joint Committee on Cancer (72). All IBC patient tissues were reviewed by a pathologist (Savitri K.) to ensure accurate diagnosis and tumor cellularity >60%. Frozen matched tumor and normal breast tissue RNAs from 4 deidentified non-IBC patients were purchased from OriGene (CR525106 and CR560146, CR520829 and CR561033). Individual patient information, including HR and HER2 status for all MD Anderson patients and provided by the supplier for the frozen commercial samples, is summarized in Table 1.

### RNA Preparation

Total cellular RNA was extracted from FFPE tumor slices and PBMCs at M.D. Anderson by using a miR Vana miRNA isolation kit (ThermoFisher), shipped to UT Austin on dry ice, and stored frozen at −80° C until ready for use. For TGIRT-seq library preparation, the thawed tumor and PBMC RNAs (500 ng for most samples but as little as 200 ng for a few tumor samples) were incubated with Baseline-ZERO DNase (Lucigen; 2 units, 30 min at 37° C) to digest DNA followed by rRNA depletion using an Illumina RiboZero Gold (Human/Mouse/Rat) kit. The rRNA-depleted RNAs were then cleaned up with a Zymo RNA clean and concentrator kit by using the manufacturer's two-fraction protocol to separate long and short RNAs. The long RNAs (≥200 nt) were chemically fragmented to 70-90 nt length by using an NEBNext Magnesium RNA Fragmentation Module (New England Biolabs; 94° C for 5 min) and cleaned up with a Zymo RNA clean and concentrator kit using a modified 8× ethanol protocol (v/v ratio 1:2:8 for RNA sample: kit RNA Binding Buffer: 100% ethanol) to minimize loss of very small RNAs (73, 74). The chemically fragmented long RNAs were then combined with the non-chemically fragmented short RNAs (<200 nt), and the reconstituted total RNA sample was treated with T4 polynucleotide kinase (Lucigen; 50 U for 30 min at 37° C) to remove 3′ phosphates and 2′,3′-cyclic phosphates, which impede TGIRT template switching, followed by a final clean-up with a Zymo RNA clean and concentrator kit using the modified 8× ethanol protocol, as described above.

Plasma was prepared at M.D. Anderson from the same blood samples as PBMCs by sedimentation into a Ficoll cushion being careful to avoid cell breakage (2, 3) and shipped to UT Austin on dry ice. Plasma RNAs were isolated using a miRNeasy Serum/Plasma Advanced kit (Qiagen) with a modified lysis buffer step in which the plasma samples were incubated with proteinase K (20 mg/mL stock, 1% v/v) in the presence of SDS (0.5% stock, 1% v/v) and linear acrylamide carrier (5 mg/mL stock, 1-2 01 per sample) for 15 min at 50° C in order to improve the recovery of protein-bound RNAs. After neutralization, centrifugation, and column clean up as specified in the kit protocol, the isolated RNAs were treated with T4 polynucleotide kinase (Lucigen; 50 units, 30 min at 37° C) to remove 3′ phosphates and with DNase I (Zymo: 30 U, 30 min at 37° C) to degrade residual DNA, followed by a final clean up with a Zymo RNA clean and concentrator kit using the modified 8× ethanol protocol described above.

The Purchased frozen matched non-IBC tumor and healthy tissue RNAs (500 ng) from 4 non-IBC patients were treated with Baseline-ZERO DNase for 30 min at 37° C to remove residual DNA and then processed by using the Zymo kit two-fraction protocol to obtain total RNA samples with chemically fragmented long RNAs and intact short RNAs, as described above for the FFPE tumor and PBMC samples.

For all samples, RNA concentration and quality were checked by mRNA assay with a Pico Kit on an Agilent 2100 Bioanalyzer. The recovered RNAs (2-50 ng for cellular RNAs and 0.5-2 ng for plasma RNAs) were used for TGIRT-seq library preparation.

### Construction and Sequencing of TGIRT-Seq Libraries

TGIRT-seq libraries were constructed as described (73, 74) by using TGIRT-template switching from a synthetic RNA template/DNA primer duplex to the 3′ end of the target RNA for 3′ RNA-seq adapter addition and a single-stranded DNA ligation to the 3′ end of the cDNA using Thermostable 5′ AppDNA/RNA Ligase (New England Biolabs) for 5′ RNA-seq adapter addition. The TGIRT-template switching reaction was done with 1 μM TGIRT-III RT (InGex, LLC) for 15 min at 60° C). The resulting cDNAs were amplified by PCR with primers that add capture sites and indices for Illumina sequence (denaturation 98° C for 5 sec, followed by 12 cycles of 98° C for 5 sec, 65° C for 10 sec, and 72° C for 10 sec) The PCR products were purified by using Agencourt AMPure XP beads (Beckman Coulter), and the libraries were sequenced on an Illumina NextSeq 500 to obtain 2×75-nt paired-end reads.

### Read Mapping

Illumina TruSeq adapters and PCR primer sequences were trimmed from the reads with Cutadapt v2.8 (75) (sequencing quality score cut-off at 20; p-value<0.01) and reads<15-nt after trimming were discarded. To minimize mismapping, we used a sequential mapping strategy. First, reads were mapped to the human mitochondrial genome (Ensembl GRCh38 Release 93) and the Escherichia coli genome (GeneBank: NC_000913) using HISAT2 v2.1.0 (76) with customized settings (-k 10--rfg 1,3--rdg 1,3--mp 4,2--no-mixed--no-discordant--no-spliced-alignment) to identify reads derived from mitochondrial and E. coli RNAs (denoted Pass 1). Unmapped reads from Pass1 were then mapped to a collection of customized references sequences for human sncRNAs (miRNA, tRNA, Y RNA, Vault RNA, 7SL RNA, 7SK RNA) and rRNAs (2.2-kb 5S rRNA repeats from the 5S rRNA cluster on chromosome 1 (1q42, Gene-Bank: X12811) and 43-kb 45S rRNA containing 5.8S, 18S and 28S rRNAs from clusters on chromosomes 13,14,15, 21, and 22 (GeneBank: U13369), using HISAT2 with the following settings-k 20--rdg 1,3--rfg 1,3--mp 2,1--no-mixed--no-discordant--no-spliced-alignment--norc (denoted Pass 2). Unmapped reads from Pass 2 were then mapped to the human genome reference sequence (Ensembl GRCh38 Release 93) using HISAT2 with settings optimized for nonspliced mapping (-k 10--rdg 1,3--rfg 1,3--mp 4,2--no-mixed--no-discordant--no-spliced-alignment) (denoted Pass 3) followed by splice aware mapping (-k 10 --rdg 1,3--rfg 1,3--mp 4,2--no-mixed--no-discordant--dta) (denoted Pass 4). For reads that mapped to multiple genomic loci with the same mapping score in passes 3 to 4, the alignment with the shortest distance between the two paired ends (i.e., the shortest read span) was selected. In the case of ties (i.e., reads with the same mapping score and read span), reads mapping to a chromosome were selected over reads mapping to scaffold sequences, and in other cases, the read was assigned randomly to one of the tied choices. The filtered multiply mapped reads were then combined with the uniquely mapped reads from Passes 3-4 by using SAMtools v1.10 (77).

To generate counts for different genomic features, the mapped reads were intersected with Ensembl GRCh38 Release 93 gene annotations plus the RNY5 gene and its 10 pseudogenes, which were not annotated in this release. Annotations for repeat elements were from RepBase, and annotations for hairpin miRNA and mature miRNA were from miRbase, each feature was calculated by BEDTools v2.29.2. To avoid miscounting reads with embedded sncRNAs that were not filtered out in Pass 2 (e.g., snoRNAs), reads were first intersected with sncRNA annotations, and the remaining reads were then intersected with the annotations for protein-coding gene RNAs, lincRNAs, antisense RNAs, and other lncRNAs to get the read count for each annotated feature. Reads coverages were calculated from merged BAM files and converted to BigWig format using UCSC commandline utility tools, and then visualized by using Integrative Genomics Viewer v2.6.2 (IGV).

### Normalization and Differential Expression Analysis

Read counts from tumor tissue samples were normalized by using DESeq2 (42) with sample size factors estimated based on counts from nuclear protein-coding genes. Genewise differential expression analyses were also performed using DESeq2, while all other analyses were applied to log-transformed DESeq2-normalized expression values (with log 2 transformation applied after adding a pseudocount of 1 to avoid the singular behavior of log 20). Unless noted otherwise, the phrase “significantly DE” indicates that the genes in question met the stringent criteria of adj. p≤0.001 and |LFC|≥2 according to DESeq2 analysis. Candidate genes were checked visually by IGV to see if differential expression reflected read coverage across the gene or was limited to specific regions and to filter out protein-coding, lncRNA, and miRNA genes whose differential expression was due to reads mapping to simple repeats or other low complexity sequences as defined by RepBase.

### Identification of Gene Sets Enriched for Differential Expression

To get a better sense of themes underlying the patterns of DE genes, we looked for evidence of enrichment of either elevated or depressed DESeq2 test statistics among genes composing either the BROAD Institute's list of hallmark gene sets (78) or Gene Ontology (GO) biological process gene sets (79). GO-MWU (Gene Ontology Mann-Whitney U testing, (80), which analyzes each GO category G within specified size limits (e.g., minimum of 5 observed genes, maximum of 5% of all observed genes) and tests whether the scores (e.g., DESeq2 test statistics) associated with genes within G were significantly more likely to be found near the top or bottom of the global ranked list of genes than would randomly shuffled genes.

Because GO-MWU relies on the MWU test, it does not require specification of an arbitrary threshold for identification of significant genes, and it is also able to take advantage of ordinal information that some genes are more significantly DE than others. GO-MWU handles another common problem in gene-set enrichment analysis—how to deal with the hierarchical and overlapping nature of GO categories—through a clustering approach that merges GO categories with highly overlapping sets of associated genes and retains the name of the largest merged category. Here this was specifically done by applying a dendrogram cut height of 0.25: since GO-MWU defines gene set dissimilarities by the ratio of the number of overlapping genes to the number of genes in the smaller gene set, application of complete-linkage clustering merges together a cluster of gene sets only when the most dissimilar pair of them overlaps in ≥75% of genes contained in the smaller of the pair.

### Peak Calling

For each sample, a BAM file with read pairs that mapped to the human-genome was processed as described above. Reads that overlapped sncRNAs (Ensembl annotation), rRNA loci, Mt DNA (to eliminate mis-mapping of Mt RNAs to nuclear Mt DNA segments (NUMTs), and human genome blacklist regions were removed using BEDTools. BAM files from libraries within the same group (Healthy, non-IBC, and IBC) were combined and then split into separate BAM files containing only reads mapping to the forward or reverse strand. BAM files for each strand were then used as input for the MACS2 callpeak algorithm using the combined BAM file for both strands from healthy or non-IBC plasma datasets (n=15 and 6, respectively) as the base-line control, via: macs2 callpeak--treatment {combined BAM from IBC dataset, forward or reverse strand}--control {combined BAM from Healthy or non-IBC dataset, forward or reverse strand}--outdir {outdir}--name {comparison_strand}--nomodel--format BAMPE--qvalue 0.01--keep-dup all--gsize hs--min-length 150.

The called peaks from the forward and reverse strands were then combined and filtered to obtain candidate peaks with read coverage≥10 reads at the peak maximum, a false discovery rate≤0.01 (q-value assigned by MACS2), and a requirement that the peak be detected in at least 5 of the 10 IBC plasma samples to avoid batch effects.

The annotation of peaks was done with BEDTools intersect command against genomic features from Ensembl genes and RepBase-annotated repeats. An overlap score, defined as the number of bases overlapping the genomic feature divided by the peak width, was computed for each overlapping record between a peak and a genomic feature. A maximal overlap score of 1 indicates the peak can be explained by the full-length genomic feature. A best feature annotation for each peak was selected by the highest overlap score. In cases of identical overlap scores, a feature was selected with the priority: repeat regions (RepBase), protein-coding genes, lncRNAs.

### Intron-Exon Depth Ratio (IDR) Analysis

We consider IDR, both as a single quantity θ representing all genes and separately as an individual quantity θ_{g} for each gene, through a simple model y=θx/[(θ−1)x+1], where y is the estimated fraction of reads for a protein-coding gene in FFPE IBC tumor samples derived from introns and x is the fraction of the genomic annotation of the gene's length corresponding to introns. This model arises from the assumption that the per-base rate of intronic region inclusion in sequencing reads is a fixed quantity θ times the per-base exonic region inclusion rate. The intronic read fractions y_{g} for gene g are estimated as 1−(# reads mapped to g using transcriptomic reference/# reads mapped to g using genomic reference), capped at a maximum value of 1 and floored at a minimum value of 0. Mapping to the transcriptomic reference was done using HISAT2 and Ensembl GRCh38 Release 93 reference, while genomic mapping was performed as described in read mapping section above. Genomic and transcriptomic mapping results included slightly different gene sets; we assigned IDR values only to genes included in both results sets.

All-genes θ (with no subscript) values were estimated by least-squares using data for all genes; in the FFPE IBC sample set, for example, this yields a value of ~0.19, indicating that read depth for intronic regions tends to be about 0.19 times the read depth for the exonic regions on average across all genes. However, some genes have much higher or lower θ_{g} (note subscript g) values: IDR θ_{g} for a given gene g is defined as the value of θ(g) which solves y_{g}=θx_{g}/[(θ−1)x_{g}+1] for the values y_{g} and x_{g} associated specifically with gene g only.

IDR values for each gene were calculated separately for each combination of diagnostic group (healthy, non-IBC, and IBC) and sample type (frozen tissue, FFPE, and PBMC). Asterisks are used to mark genes of interest with IDR≥0.5 in either FFPE IBC sample set or PBMC IBC sample set, as appropriate to the analysis discussed.

### Tables

TABLE 1
Donor Information

| [symbol] ID | Sample | [symbol] type ( [symbol] ID) | | | | | | | | | | |
|---|---|---|---|---|---|---|---|---|---|---|---|---|
| [symbol] | ID | [symbol] | [symbol] | [symbol] | [symbol] | Age | Race | [symbol] | [symbol] | [symbol] | [symbol] | [symbol] |
| Healthy 1 | [symbol] | | | [symbol] | [symbol] | | | | | | | |
| Healthy 2 | [symbol] | | | [symbol] | [symbol] | | | | | | | |
| Healthy 3 | [symbol] | | | [symbol] | [symbol] | | | | | | | |
| Healthy 4 | [symbol] | | | [symbol] | [symbol] | | | | | | | |
| Healthy 5 | [symbol] | | | [symbol] | [symbol] | | | | | | | |
| Healthy 6 | [symbol] | | | [symbol] | [symbol] | | | | | | | |
| Healthy 7 | [symbol] | | | [symbol] | [symbol] | | | | | | | |
| Healthy 8 | [symbol] | | | [symbol] | [symbol] | | | | | | | |
| Healthy 9 | [symbol] | | | [symbol] | [symbol] | | | | | | | |
| Healthy 10 | [symbol] | | | [symbol] | [symbol] | | | | | | | |
| Healthy 11 | [symbol] | | | [symbol] | [symbol] | | | | | | | |
| Healthy 12 | [symbol] | | | [symbol] | [symbol] | | | | | | | |
| Healthy 13 | [symbol] | | | [symbol] | [symbol] | | | | | | | |
| Non-IBC 1 | [symbol] | | [symbol] | [symbol] | [symbol] | 55 | White | Negative | Negative | Negative | Negative | N |
| | [symbol] | | | | | | | | | | | |
| | [symbol] | | | | | | | | | | | |
| Non-IBC 2 | [symbol] | | [symbol] | [symbol] | [symbol] | 61 | White | Positive | Positive | Negative | Positive | N |
| | [symbol] | | | | | | | | | | | |
| | [symbol] | | | | | | | | | | | |
| Non-IBC 3 | [symbol] | | | [symbol] | [symbol] | [symbol] | White | Positive | Positive | Negative | Positive | N |
| | [symbol] | | | | | | | | | | | |
| Non-IBC 4 | [symbol] | | [symbol] | [symbol] | [symbol] | [symbol] | White | Negative | Negative | Negative | Negative | N |
| | [symbol] | | | | | | | | | | | |
| | [symbol] | | | | | | | | | | | |
| Non-IBC 5 | [symbol] | | | [symbol] | [symbol] | [symbol] | White | Negative | Negative | Negative | Negative | N |
| | [symbol] | | | | | | | | | | | |
| Non-IBC 6 | [symbol] | | [symbol] | [symbol] | [symbol] | 49 | White | Positive | Positive | Positive | Positive | N |
| | [symbol] | | | | | | | | | | | |
| | [symbol] | | | | | | | | | | | |
| IBC 1 | [symbol] | | [symbol] | [symbol] | [symbol] | [symbol] | White | Positive | Negative | Negative | Positive | Y |
| | [symbol] | | | | | | | | | | | |
| | [symbol] | | | | | | | | | | | |
| IBC 2 | [symbol] | | [symbol] | [symbol] | [symbol] | 66 | White | Negative | Negative | Negative | Negative | Y |
| | [symbol] | | | | | | | | | | | |
| | [symbol] | | | | | | | | | | | |
| IBC 3 | [symbol] | | [symbol] | [symbol] | [symbol] | [symbol] | White | Negative | Negative | Negative | Negative | Y |
| | [symbol] | | | | | | | | | | | |
| | [symbol] | | | | | | | | | | | |
| IBC 4 | [symbol] | | [symbol] | [symbol] | [symbol] | [symbol] | White | Positive | Positive | Negative | Positive | Y |
| | [symbol] | | | | | | | | | | | |
| | [symbol] | | | | | | | | | | | |
| IBC 5 | [symbol] | | [symbol] | [symbol] | [symbol] | 23 | White | Positive | Negative | Negative | Positive | Y |
| | [symbol] | | | | | | | | | | | |
| | [symbol] | | | | | | | | | | | |
| IBC 6 | [symbol] | | [symbol] | [symbol] | [symbol] | [symbol] | White | Positive | Positive | Negative | Positive | Y |
| | [symbol] | | | | | | | | | | | |
| | [symbol] | | | | | | | | | | | |
| IBC 7 | [symbol] | | [symbol] | [symbol] | [symbol] | [symbol] | White | Positive | Positive | Negative | Positive | Y |
| | [symbol] | | | | | | | | | | | |
| | [symbol] | | | | | | | | | | | |
| IBC 8 | [symbol] | | [symbol] | [symbol] | [symbol] | [symbol] | White | Positive | Positive | Negative | Positive | Y |
| | [symbol] | | | | | | | | | | | |
| | [symbol] | | | | | | | | | | | |
| IBC 9 | [symbol] | | [symbol] | [symbol] | [symbol] | [symbol] | White | Positive | Negative | Negative | Positive | Y |
| | [symbol] | | | | | | | | | | | |
| | [symbol] | | | | | | | | | | | |
| IBC 10 | [symbol] | | [symbol] | [symbol] | [symbol] | [symbol] | White | Positive | Negative | Negative | Positive | Y |
| | [symbol] | | | | | | | | | | | |
| | [symbol] | | | | | | | | | | | |
| Healthy A | [symbol] | [symbol] | | | | 47 | White | | | | | |
| Healthy B | [symbol] | [symbol] | | | | [symbol] | Unknown | | | | | |
| Healthy C | [symbol] | [symbol] | | | | 57 | White | | | | | |
| Healthy D | [symbol] | [symbol] | | | | [symbol] | White | | | | | |
| Non-IBC A | [symbol] | [symbol] | | | | 47 | White | Negative | Unknown | Negative | Unknown | N |
| Non-IBC B | [symbol] | [symbol] | | | | 63 | Unknown | Positive | Positive | Negative | Positive | N |
| Non-IBC C | [symbol] | [symbol] | | | | 57 | White | Positive | Positive | Negative | Positive | N |
| Non-IBC D | [symbol] | [symbol] | | | | [symbol] | White | Negative | Unknown | Negative | Unknown | N |

*Sample ID for [symbol]
**Sample ID for [symbol]
***Sample ID for

[symbol]

[symbol]

[symbol]

indicates data missing or illegible when filed

TABLE 2A-C
Intron Depth Ratio Summary Statistics
(A) Overal IDR Percentile*

| Group | 25^{th} | 50^{th} (Median) | 75^{th} | % IDR ≥0.5 |
|---|---|---|---|---|
| FT Healthy | 0.06 | 0.14 | 0.28 | 13.9 |
| FT non-IBC | 0.05 | 0.10 | 0.23 | 12.3 |
| FFPE non-IBC | 0.09 | 0.20 | 0.43 | 19.5 |
| FFPE IBC | 0.09 | 0.19 | 0.39 | 18.4 |
| PBMC Heathy | 0.05 | 0.10 | 0.26 | 14.6 |
| PBMC non-IBC | 0.06 | 0.14 | 0.35 | 17.3 |
| PBMC IBC | 0.10 | 0.23 | 0.55 | 24.6 |
| Plasma Healthy | 0.10 | 0.37 | >10 | 36.5 |
| Plasma non-IBC | 0.02 | 0.23 | >10 | 30.0 |
| Plasma IBC | 0.18 | 1.34 | >10 | 48.7 |

(B) Overexpressed (OE) Protein-coding Genes, p ≤ 0.001, LFC ≥ 2.

| Comparison | OE Genes: #** | OE Genes w/IDR ≥0.5: # (%) | |
|---|---|---|---|
| Frozen non-IBC vs. Healthy | 20 | 2 | (10.0) |
| Frozen Healthy vs. non-IBC | 148 | 17 | (11.5) |
| FFPE IBC vs. non-IBC | 28 | 5 | (17.9) |
| FFPE non-IBC vs. IBC | 0 | — | |
| PBMC IBC vs. Healthy | 498 | 297 | (59.6) |
| PBMC Healthy vs. IBC | 32 | 5 | (15.6) |
| PBMC IBC vs. non-IBC | 194 | 120 | (61.9) |
| PBMC non-IBC vs. IBC | 3 | 0 | (0) |
| PBMC non-IBC vs. Healthy | 0 | — | |
| PBMC Healthy vs. non-IBC | 3 | 3 | (100) |
| Plasma IBC vs. Healthy | 65 | 37 | (56.9) |
| Plasma Healthy vs. IBC | 126 | 29 | (23.0) |
| Plasma IBC vs. non-IBC | 86 | 55 | (64.0) |
| Plasma non-IBC vs. IBC | 11.5 | 14 | (12.2) |
| Plasma non-IBC vs. Healthy | 41 | 15 | (36.6) |
| Plasma Healthy vs. non-IBC | 45 | 21 | (45.7) |

(C) OE Protein-coding Genes, p ≤ 0.05, LFC ≥ 1.

| Comparison | OE Genes: #** | OE Genes w/IDR ≥ 0.5: # (%) | |
|---|---|---|---|
| Frozen non-IBC vs. Healthy | 272 | 10 | (3.7) |
| Frozen Healthy vs. non-IBC | 552 | 49 | (8.7) |
| FFPE IBC vs. non-IBC | 204 | 55 | (27.0) |
| FFPE non-IBC vs. IBC | 10 | 0 | (0) |
| PBMC IBC vs. Healthy | 2346 | 975 | (41.6) |
| PBMC Healthy vs. IBC | 1262 | 163 | (12.9) |
| PBMC IBC vs. non-IBC | 1268 | 578 | (45.6) |
| PBMC non-IBC vs. IBC | 368 | 43 | (11.7) |
| PBMC non-IBC vs. Healthy | 29 | 8 | (27.6) |
| PBMC Healthy vs. non-IBC | 153 | 17 | (11.1) |
| Plasma IBC vs. Healthy | 280 | 192 | (68.6) |
| Plasma Healthy vs. IBC | 406 | 111 | (27.3) |
| Plasma IBC vs. non-IBC | 286 | 212 | (74.1) |
| Plasma non-IBC vs. IBC | 416 | 43 | (10.3) |
| Plasma non-IBC vs. Healthy | 49 | 16 | (32.7) |
| Plasma Healthy vs. non-IBC | 80 | 37 | (46.2) |

*IDR Percentiles are values for which 25, 50, or 75%, repectively, of protein-coding genes have an IDR ≤ indicated number.
**Overexpressed gene numbers here include ony those genes for which IDR could be assigned by comparing genomic and and transcriptomic mapping results.

TABLE 3
IBC Markers in Tumors

| | | | |
|---|---|---|---|
| (ATGAA)n | EGFR | miR-125b-5p | SMIM3 |
| (ATTCC)n | EGR1 | miR-15b-5p | SNORA21 |
| (CTCT)n | F3 | miR-199a-5p | SNORD123 |
| (TCCATTCCAT)n | FGFBP1 | miR-24-3p | SNORD44 |
| (TGAGA)n | FKBP5 | MT_ATP8 | SPATA18 |
| AC006924.1 | FOS | NEXN | STX1B |
| AC036176.3 | FOXC2 | NFIA_AS2 | TAT |
| AC044784.1 | GEM | NR4A1 | TNFAIP3 |
| ALDH1A1 | JUN | PER1 | ZBTB16 |
| CD24 | KLF9 | PIGR | |
| COL4A6 | L1M3b | RARRES1 | |
| CSRNP1 | let-7b-5p | RGCC | |
| CTGF | LINC02015 | RGS1 | |
| CYR61 | LINC02562 | SEPT7P6 | |
| DUSP1 | miR-125a-5p | SLC6A14 | |

TABLE 4
IBC Markers in PBMCs

| | | | | |
|---|---|---|---|---|
| (AGAT)n | BSR/Beta | miR-19b-3p | NEAT1 | SNORD41 |
| (AGGGTT)n | C5orf17 | miR-20a-5p | NEK7 | SNORD72 |
| (CATC)n | FAM133DP | MIR21 | NFKB1 | SNORD98 |
| (CCATT)n | G2E3_AS1 | miR-21-5p | NR2F2_AS1 | TRA-AGC- |
| | | | | 3′half |
| (GATG)n | GCSHP3 | MIR221 | OLR1 | TRA-CGC- |
| | | | | 3′half |
| (GGGTTA)n | L1PB | miR-221-3p | PRIMA41-int | TRA-TGC- |
| | | | | 3′half |
| (GTCC)n | let-7a-5p | MIR222 | PTCHDT_AS | TRE-CTC- |
| | | | | 5′half |
| (GTTAGG)n | let-7f-5p | MIR223 | RNU6V | TRG-CCC |
| (TCCG)n | let-7i-5p | miR-223-3p | SAMSN1 | TRG-CCC- |
| | | | | 3′half |
| (TTAGGG)n | LINC00276 | miR-24-3p | SCARNA14 | TRG-GCC- |
| | | | | 3′half |
| AC005323.2 | LINC01192 | miR-25-3p | SCARNA15 | TRH-GTG |
| AC007402.1 | LINC01830 | miR-26a-5p | SCARNA4 | TRH-GTG- |
| | | | | 3′half |
| AC009264.1 | LINC01924 | miR-27a-3p | SCARNA8 | TRI-AAT- |
| | | | | tRF3 |
| AC010967.1 | LTR89 | miR-29a-3p | SDC2 | TRK-TTT- |
| | | | | 5′half |
| AC011306.1 | MALAT1 | miR-29b-3p | SERPINB2 | TRM-CAT |
| AC018697.1 | MGAM | miR-29c-3p | SNORA105A | TRN-GTT- |
| | | | | tRF3 |
| AC019117.2 | MIR103A1 | miR-30b-5p | SNORA11 | TRP-AGG- |
| | | | | 3′half |
| AC021351.1 | MIR103A2 | MIR30C1 | SNORA13 | TRP-CGG |
| AC022639.1 | miR-103a-3p | miR-30c-5p | SNORA15B | TRP-CGG- |
| | | | | 3′half |
| AC087565.3 | miR-125a-5p | MIR30D | SNORA16A | TRP-TGG- |
| | | | | 3′half |
| AC091862.1 | miR-126-3p | miR-30d-5p | SNORA19 | TRQ-CTG |
| AC099520.1 | miR-126-5p | MIR30E | SNORA28 | TRQ-CTG- |
| | | | | 3′half |
| AC103409.1 | MIR142 | miR-30e-5p | SNORA2B | TRQ-TTG- |
| | | | | 3′half |
| AC106798.1 | miR-142-3p | MIR325HG | SNORA32 | TRR-ACG |
| AC114316.2 | miR-142-5p | miR-338-3p | SNORA36A | TRR-CCT |
| AC122718.1 | miR-146a-5p | miR-342-3p | SNORA36B | TRR-TCT |
| AC130895.1 | miR-146b-5p | MIR4300HG | SNORA3B | TRR-TCT- |
| | | | | 3′half |
| AC246680.1 | miR-148a-3p | miR-454-3p | SNORA3C | TRS-AGA- |
| | | | | 3′half |
| ADAMTS9_AS2 | MIR150 | MIR650 | SNORA41 | TRS-CGA- |
| | | | | 3′half |
| AF279873.3 | miR-151a-3p | miR-98-5p | SNORA46 | TRS-TGA- |
| | | | | 3′half |
| AL354793.1 | miR-151a-5p | MIRLET7A1 | SNORA53 | TRT-AGT- |
| | | | | 3′half |
| AL357507.1 | miR-15a-5p | MIRLET7A2 | SNORA5A | TRT-CGT- |
| | | | | 3′half |
| AL359313.1 | MIR 15B | MIRLET7C | SNORA65 | TRT-TGT- |
| | | | | 3′half |
| AL445250.1 | miR-15b-5p | MIRLET7F1 | SNORA69 | TRU-TCA- |
| | | | | tRF3 |
| AL513323.1 | MIR16-1 | MIRLET7F2 | SNORA75 | TRV-AAC |
| ALR/Alpha | MIR16-2 | MIRLET7G | SNORA81 | TRV-AAC- |
| | | | | 3′half |
| AP000769.1 | miR-16-5p | MIR185 | SNORD101 | TRV-CAC- |
| | | | | 3′half |
| AP005203.1 | miR-185-5p | MIR26B | SNORD12C | TRV-TAC |
| ATP2B1 | miR-199a-5p | MLT1I-int | SNORD2 | TRW-CCA |
| BRAF | miR-19a-3p | NAMPT | SNORD28 | TRX-CAT |
| VNN3 | | | | |

TABLE 5
IBC Markers in Plasma

| | | | |
|---|---|---|---|
| (AAGA)n | CLSTN2 | L1PA7 | MIRLET7F2 |
| (AGAA)n | CMSS1 | L2a | MLT1A0 |
| (CATTC)n | DGKB | L2c | MLT1A1 |
| (CT)n | DIRAS3 | let-7a-5p | MLT1J |
| (TCCT)n | FAM135B | LINC01191 | MLT1K |
| (TTC)n | GA-rich | LINC01495 | MLT2F |
| AC004014.1 | GLIS3 | LUZP2 | MSTA-int |
| AC096759.1 | HAL1 | MER101B | NADK2 |
| AC117373.1 | KCNH5 | MER106A | NLGN1 |
| AC131254.1 | L1Hs | MER4B-int | NUP210L |
| ADAMTS6 | L1M5 | MER65C | PABL_B |
| ADAMTSL1 | L1MA9 | METTL15 | PHF24 |
| ADGRV1 | L1MB3 | MIOS | PLVAP |
| ALR/Alpha | L1MB7 | MIR | RNY5 |
| AluJb | L1MC1 | miR-1246 | SAR |
| AluJr | L1MC4a | MIR1273A | SENP2 |
| AluSx | L1MCa | MIR1285-1 | SPOCK3 |
| AluSx1 | L1ME4a | miR-15a-5p | SVA_B |
| AluSx4 | L1MEc | miR-19b-3p | THE1B |
| AluY | L1MEg | MIR223 | THE1B-int |
| ANKS1B | L1P1 | MIR3 | TRBJ1-1 |
| A-rich | L1PA12 | MIR3929 | TRBJ1-2 |
| BOLL | L1PA2 | MIR5096 | TRBJ1-6 |
| C6orf118 | L1PA3 | MIR619 | UVRAG |
| CCDC150 | L1PA4 | MIRb | WWP2 |
| Charlie3 | L1PA5 | MIRc | XXYLT1 |
| CHCHD6 | L1PA6 | MIRLET7C | |

TABLE 6
Non-IBC Markers

| | | | |
|---|---|---|---|
| ICAM3 | RHOC | TRA2B | FAM212B_AS1 |

TABLE 7
Other IBC Markers

| | LFC | adj.p | LFC(IBCvs | adj.p(IBCvs | LFC | adj.p |
|---|---|---|---|---|---|---|
| ID | (IBCvsHealthy) | (IBCvsHealthy) | non-IBC) | non-IBC) | (IBCvsOther | (IBCvsOther |
| (AATT)n | 23.8978694 | 4.12744E-12 | 26.4856452 | 7.49089E−10 | 24.26742586 | 2.19152E−16 |
| (ACTC)n | 22.33467708 | 1.82379E−09 | 23.40334994 | 5.39043E−07 | 22.51039266 | 5.39537E−11 |
| (GCTCT)n | 21.98363014 | 3.47474E−09 | 24.29563823 | 1.73538E−07 | 0 | |
| (TCAATAT)n | 21.17157645 | 1.55939E−08 | 27.59290675 | 1.40426E−09 | 21.64230078 | 3.76004E−10 |
| AC026704.1 | 22.7930698 | 1.58837E−10 | 27.15729079 | 1.03658E−09 | 23.22994372 | 1.46913E−13 |
| AC084064.1 | 21.75021326 | 4.1831E−09 | 25.32100978 | 4.49508E−08 | 22.11388252 | 8.57158E−11 |
| AC136188.1 | 22.93686535 | 6.08576E−11 | 2.440943635 | 0.811057345 | 21.86245313 | 8.97429E−12 |
| AC137770.1 | 3.892506535 | 0.551443573 | 6.834058136 | 0.328792638 | 23.02761421 | 2.39274E−13 |
| AC243547.3 | 21.77752963 | 6.82454E−09 | 27.40426468 | 3.39004E−09 | 22.2217441 | 1.01942E−10 |
| AL157884.3 | 22.91155846 | | 2.759143618 | | 23.93600014 | 5.19959E−14 |
| AL161782.1 | 21.39011029 | 8.18004E−09 | 26.72855011 | 5.61593E−09 | 21.72766392 | 2.09177E−10 |
| AL353747.2 | 23.44976157 | 1.5469E−10 | 27.22924254 | 2.5889E−09 | 23.7041895 | 2.18097E−12 |
| AL358473.2 | 22.63243459 | 4.98929E−12 | 26.7434441 | 7.58643E−11 | 23.06527811 | 1.36568E−15 |
| AL445218.1 | 23.16443937 | 7.40787E−13 | 3.066395228 | 0.722922193 | 24.19541277 | 7.61629E−17 |
| AL445686.2 | 24.81067775 | 2.8467E−13 | 30.89830947 | 3.13551E−13 | 25.38134605 | 1.96092E−17 |
| ALDH4A1 | 22.53702438 | 1.58586E−09 | 27.91880922 | 1.54333E−09 | 3.85109028 | |
| ATOH8 | 24.5385131 | 2.57665E−11 | 29.06787399 | 2.86491E−10 | 25.00989114 | 1.2106E−13 |
| C16orf89 | 23.11160251 | 2.69101E−12 | 3.252740934 | 0.683223935 | 24.12714325 | 4.05461E−16 |
| CBLN4 | 21.66903927 | 8.28546E−09 | 23.71175867 | 7.02051E−07 | 21.95285318 | 1.80467E−10 |
| CDC42EP1 | 24.62810656 | 2.14105E−11 | 29.75908812 | 1.0662E−10 | 25.06000894 | 1.09059E−13 |
| CNTN2 | 20.97511128 | | 0.292236049 | | 22.02934941 | 1.52999E−10 |
| CSF2RBP1 | 21.27352894 | | 24.36873882 | | 21.55879627 | 3.02092E−10 |
| DUX4 | 22.91782146 | 1.29818E−11 | 26.27766845 | 6.58431E−10 | 23.34381291 | 6.57302E−16 |
| HHIP_AS1 | 22.67707134 | 7.12856E−10 | 26.5808277 | 6.8605E−09 | 23.10232065 | 9.16165E−12 |
| ITPKB_IT1 | 22.16229279 | 1.90958E−09 | 25.15491074 | 5.66584E−08 | 22.56559086 | 3.0707E−11 |
| L1HS | 3.569312631 | 0.000260196 | 4.741253535 | 3.45098E−05 | 3.846626094 | 1.24543E−05 |
| LINC00463 | 5.36216396 | 0.396671145 | 21.84012605 | 5.06793E−06 | 22.28671251 | 5.84568E−11 |
| LINC00619 | 25.28902126 | 4.15569E−12 | 27.0482238 | 3.39308E−09 | 24.4600683 | 6.21752E−14 |
| LINC01943 | 4.480038455 | 0.477357798 | 22.85453066 | 3.83533E−07 | 23.41919804 | 1.46913E−13 |
| LINC02300 | 21.58584738 | 5.64159E−09 | 24.08088005 | 2.47298E−07 | 21.13399298 | 7.51052E−10 |
| LINC02397 | 22.30102821 | 1.49697E−09 | 25.76509736 | 2.37219E−08 | 22.7263748 | 2.14851E−11 |
| LIX1 | 23.00466078 | 6.33952E−10 | 27.83732103 | 1.7398E−09 | 23.25883554 | 9.60582E−12 |
| MIR4773-1 | 20.84406551 | 2.04216E−08 | 30.23551318 | 2.02686E−11 | 22.32123388 | 6.40629E−11 |
| MIR5588 | 5.739832072 | 3.22737E−05 | 5.293223968 | 0.002822629 | 5.558612966 | 5.97133E−06 |
| MIR566 | 22.9192852 | 5.65188E−10 | 32.79042386 | 2.92991E−13 | 23.85478448 | 1.99911E−13 |
| MRFAP1L1 | 22.34564156 | 2.2416E−09 | 24.5807239 | 2.22709E−07 | 22.73617954 | 3.01536E−11 |
| NDC80 | 22.44739905 | 1.8635E−09 | 24.3694989 | 2.8943E−07 | 22.78571286 | 2.7255E−11 |
| PAX9 | 20.9025822 | | 1.371008352 | | 21.93172687 | 1.86334E−10 |
| PPP1R1A | 22.60534074 | 1.43336E−09 | 28.52284564 | 6.38546E−10 | 23.1044647 | 1.36489E−11 |
| SYT6 | 22.93544287 | 2.04598E−11 | 27.1051082 | 2.80623E−10 | 23.32951822 | 1.44964E−15 |
| THEG | 2.790026773 | 0.676847786 | 21.26741311 | 1.59393E−05 | 21.8863584 | 2.03989E−10 |
| WASH4P | 5.314355223 | 0.265951603 | 8.510583367 | 0.124727809 | 23.24373088 | 4.99592E−18 |
| XPA | 23.1869533 | 7.63509E−12 | 27.17176047 | 1.85321E−10 | 23.62738966 | 3.55642E−16 |
| (ATGAA)n | | | | | | |
| TRBJ1-n | | | | | | |
| TRBJ1-1 | 23.18028689 | 5.70041E−12 | 26.31811631 | 5.25097E−10 | 22.47980798 | 5.54903E−11 |
| TRBJ1-2 | 7.578379299 | 9.60289E−05 | 3.785284167 | 0.210402573 | 4.965490325 | 0.014014571 |
| TRBJ1-3 | 0 | | 0 | | 0 | |
| TRBJ1-4 | 0.06125439 | | 0.466289588 | | 0 | |
| TRBJ1-5 | 7.046053747 | 0.064594197 | 23.3242062 | 5.00587E−11 | 7.764140712 | 0.009503157 |
| TRBJ1-6 | 11.56011048 | 4.03992E−13 | 11.92771773 | 1.42725E−08 | 11.69289195 | 8.29744E−17 |

### REFERENCES

1. R. Stark, M. Grzelak, J. Hadfield, RNA sequencing: the teenage years. Nat Rev Genet. 20, 631-656 (2019).
2. Y. Qin, J. Yao, D. C. Wu, R. M. Nottingham, S. Mohr, S. Hunicke-Smith, A. M. Lambowitz, High-throughput sequencing of human plasma RNA by using thermostable group II intron reverse transcriptases. RNA. 22 (2016), pp. 111-128.
3. J. Yao, D. C. Wu, R. M. Nottingham, A. M. Lambowitz, Identification of protein-protected mRNA fragments and structured excised intron RNAs in human plasma by TGIRT-seq peak calling. Elife. 9 (2020), p. e60743.
4. M. Cristofanilli, A. U. Buzdar, G. N. Hortobagyi, others, Update on the management of inflammatory breast cancer. The Oncologist. 8 (2003), pp. 141-148.
5. K. W. Hance, W. F. Anderson, S. S. Devesa, H. A. Young, P. H. Levine, Trends in inflammatory breast carcinoma incidence and survival: the surveillance, epidemiology, and end results program at the National Cancer Institute. Journal of the National Cancer Institute. 97 (2005), pp. 966-975.
6. I. A. Jaiyesimi, A. U. Buzdar, G. Hortobagyi, Inflammatory breast cancer: a review. Journal of Clinical Oncology. 10 (1992), pp. 1014-1024.
7. H. Yamauchi, N. T. Ueno, Targeted therapy in inflammatory breast cancer. Cancer. 116 (2010), pp. 2758-2759.
8. H. Yamauchi, W. A. Woodward, V. Valero, R. H. Alvarez, A. Lucci, T. A. Buchholz, T. Iwamoto, S. Krishnamurthy, W. Yang, J. M. Reuben, G. N. Hortobagyi, N. T. Ueno, Inflammatory breast cancer: what we know and what we need to learn. The Oncologist. 17 (2012), p. 891.
9. H. Masuda, T. M. Brewer, D. D. Liu, T. Iwamoto, Y. Shen, L. Hsu, J. S. Willey, A. M. Gonzalez-Angulo, M. Chavez-MacGregor, T. M. Fouad, W. A. Woodward, J. M. Reuben, V. Valero, R. H. Alvarez, G. N. Hortobagyi, N. T. Ueno, Long-term treatment efficacy in primary inflammatory breast cancer by hormonal receptor- and HER2-defined subtypes. Annals of Oncology. 25 (2014), pp. 384-391.
10. N. M. Rueth, H. Y. Lin, I. Bedrosian, S. F. Shaitelman, N. T. Ueno, Y. Shen, G. Babiera, Underuse of trimodality treatment affects survival for patients with inflammatory breast cancer: an analysis of treatment and survival trends from the National Cancer Database. Journal of Clinical Oncology. 32 (2014), p. 2018.
11. J. A. Schlichting, A. S. Soliman, C. Schairer, D. Schottenfeld, S. D. Merajver, Inflammatory and non-inflammatory breast cancer survival by socioeconomic position in the Surveillance, Epidemiology, and End Results database, 1990-2008. Breast Cancer Res Treat. 134, 1257-1268 (2012).
12. S. Van Laere, I. Van der Auwera, G. G. Van den Eynden, S. B. Fox, F. Bianchi, A. L. Harris, P. Van Dam, E. A. Van Marck, P. B. Vermeulen, L. Y. Dirix, Distinct molecular signature of inflammatory breast cancer by cDNA microarray analysis. Breast Cancer Research and Treatment. 93 (2005), pp. 237-246.
13. S. J. Van Laere, I. Van der Auwera, G. G. Van den Eynden, H. J. Elst, J. Weyler, A. L. Harris, P. van Dam, E. A. Van Marck, P. B. Vermeulen, L. Y. Dirix, Nuclear 11 factor-kappaB signature of inflammatory breast cancer by cDNA microarray validated by 12 quantitative real-time reverse transcription-PCR, immunohistochemistry, and nuclear factor-13 kappaB DNA-binding. Clinical Cancer Research. 12 (2006), pp. 3249-56.
14. F. Lerebours, S. Vacher, C. Andrieu, M. Espie, M. Marty, R. Lidereau, I. Bieche, NF-kappa B genes have a major role in inflammatory breast cancer. BMC Cancer. 8 (2008), p. 41.
15. X. Wang, M. E. Reyes, D. Zhang, Y. Funakoshi, A. P. Trape, Y. Gong, T. Kogawa, B. L. Eckhardt, H. Masuda, D. A. Pirman Jr, P. Yang, J. M. Reuben, W. A. Woodward, C. Bartholomeusz, G. N. Hortobagyi, D. Tripathy, N. T. Ueno, EGFR signaling promotes inflammation and cancer stem-like activity in inflammatory breast cancer. Oncotarget. 8 (2017), p. 67904.
16. S. Van Laere, I. Van der Auwera, G. Van den Eynden, P. Van Hummelen, P. Van Dam, E. Van Marck, P. B. Vermeulen, L. Dirix, Distinct molecular phenotype of inflammatory breast cancer compared to non-inflammatory breast cancer using Affymetrix-based genome-wide gene-expression analysis. British Journal of Cancer. 97 (2007), pp. 1165-1174.
17. K. Jhaveri, E. Teplinsky, D. Silvera, A. Valeta-Magara, R. Arju, S. Giashuddin, Y. Sarfraz, M. Alexander, F. Darvishian, P. H. Levine, S. Hashmi, L. Zolfaghari, H. J. Hoffman, B. Singh, J. D. Goldberg, T. Hochman, S. Formenti, F. J. Esteva, M. S. Moran, R. J. Schneider, Hyperactivated mTOR and JAK2/STAT3 pathways: molecular drivers and potential therapeutic targets of inflammatory and invasive ductal breast cancers after neoadjuvant chemotherapy. Clinical Breast Cancer. 16 (2016), pp. 113-122.
18. K. Shirakawa, M. Shibuya, Y. Heike, S. Takashima, I. Watanabe, F. Konishi, F. Kasumi, C. K. Goldman, K. A. Thomas, A. Bett, M. Terada, H. Wakasugi, Tumor-infiltrating endothelial cells and endothelial precursor cells in inflammatory breast cancer. International Journal of Cancer. 99 (2002), pp. 344-351.
19. I. Van der Auwera, S. J. Van Laere, G. G. Van den Eynden, I. Benoy, P. van Dam, C. G. Colpaert, S. B. Fox, H. Turley, A. L. Harris, E. A. Van Marck, P. B. Vermeulen, L. Y. Dirix, Increased angiogenesis and lymphangiogenesis in inflammatory versus noninflammatory breast cancer by real-time reverse transcriptase-PCR gene expression quantification. Clinical Cancer Research. 10 (2004), pp. 7965-7971.
20. L. L. C. Marotta, V. Almendro, A. Marusyk, M. Shipitsin, J. Schemme, S. R. Walker, N. Bloushtain-Qimron, J. J. Kim, S. A. Choudhury, R. Maruyama, Z. Wu, M. Gonen, L. A. Mulvey, M. O. Bessarabova, S. J. Huh, S. J. Silver, S. Y. Kim, S. Y. Park, H. E. Lee, K. S. Anderson, A. L. Richardson, T. Nikolskaya, Y. Nikolsky, X. S. Liu, D. E. Root, W. C. Hahn, D. A. Frank, K. Polyak, The JAK2/STAT3 signaling pathway is required for growth of CD44+ CD24—stem cell-like breast cancer cells in human tumors. The Journal of Clinical Investigation. 121 (2011), pp. 2723-2735.
21. B. A. Overmoyer, V. Almendro, S. Shu, G. Peluffo, S. Y. Park, F. Nakhlis, J. R. Bellon, E. D. Yeh, J. Jacene HA Hirshfield-Bartek, K. Polyak, JAK2/STAT3 activity in inflammatory breast cancer supports the investigation of JAK2 therapeutic targeting. Cancer Reseach. 72 (24 Suppl) (2012).
22. N. Cabioglu, Y. Gong, R. Islam, K. R. Broglio, N. Sneige, A. Sahin, A. M. Gonzalez-Angulo, P. Morandi, C. Bucana, G. N. Hortobagyi, M. Cristofanilli, Expression of growth factor and chemokine receptors: new insights in the biology of inflammatory breast cancer. Annals of Oncology. 18 (2007), pp. 1021-1029.
23. M. L. Alpaugh, J. S. Tomlinson, Y. Ye, S. H. Barsky, Relationship of sialyl-Lewisx/a underexpression and E-cadherin overexpression in the lymphovascular embolus of inflammatory breast carcinoma. The American Journal of Pathology. 161 (2002), pp. 619-628.
24. K. L. van Golen, S. Davies, Z. F. Wu, Y. Wang, C. D. Bucana, H. Root, S. Chandrasekharappa, M. Strawderman, S. P. Ethier, S. D. Merajver, A novel putative low-affinity insulin-like growth factor-binding protein, LIBC (lost in inflammatory breast cancer), and RhoC GTPase correlate with the inflammatory breast cancer phenotype. Clinical Cancer Research. 5 (1999), pp. 2511-2519.
25. D. Silvera, R. Arju, F. Darvishian, P. H. Levine, L. Zolfaghari, J. Goldberg, T. Hochman, S. C. Formenti, R. J. Schneider, Essential role for eIF4GI overexpression in the pathogenesis of inflammatory breast cancer. Nature Cell Biology. 11 (2009), pp. 903-908.
26. S. J. Van Laere, N. T. Ueno, P. Finetti, P. Vermeulen, A. Lucci, F. M. Robertson, M. Marsan, T. Iwamoto, S. Krishnamurthy, H. Masuda, P. van Dam, W. A. Woodward, P. Viens, M. Cristofanilli, D. Birnbaum, L. Dirix, J. M. Reuben, F. Bertucci, Uncovering the molecular secrets of inflammatory breast cancer biology: an integrated analysis of three distinct affymetrix gene expression datasets. Clinical Cancer Research. 19 (2013), pp. 4685-4696.
27. K. Kai, T. Iwamoto, D. Zhang, L. Shen, Y. Takahashi, A. Rao, A. Thompson, S. Sen, N. T. Ueno, CSF-1/CSF-1R axis is associated with epithelial/mesenchymal hybrid phenotype in epithelial-like inflammatory breast cancer. Sci Rep. 8, 9427 (2018).
28. S. Martín-Alonso, E. Frutos-Beltrán, L. Menéndez-Arias, Reverse Transcriptase: From Transcriptomics to Genome Editing. Trends Biotechnol. 39, 194-210 (2021).
29. J. L. Stamos, A. M. Lentzsch, A. M. Lambowitz, Structure of a Thermostable Group II Intron Reverse Transcriptase with Template-Primer and Its Functional and Evolutionary Implications. Mol Cell. 68, 926-939.e4 (2017).
30. M. Belfort, A. M. Lambowitz, Group II Intron RNPs and Reverse Transcriptases: From Retroelements to Research Tools. Cold Spring Harb Perspect Biol. 11, a032375 (2019).
31. S. Mohr, E. Ghanem, W. Smith, D. Sheeter, Y. Qin, O. King, D. Polioudakis, V. R. Iyer, S. Hunicke-Smith, S. Swamy, S. Kuersten, A. M. Lambowitz, Thermostable group II intron reverse transcriptase fusion proteins and their use in cDNA synthesis and next-generation RNA sequencing. RNA. 19, 958-970 (2013).
32. B. Liu, X. Dong, H. Cheng, C. Zheng, Z. Chen, T. C. Rodríguez, S.-Q. Liang, W. Xue, E. J. Sontheimer, A split prime editor with untethered reverse transcriptase and circular RNA template. Nat Biotechnol. 40, 1388-1393 (2022).
33. J. Grünewald, B. R. Miller, R. N. Szalay, P. K. Cabeceiras, C. J. Woodilla, E. J. B. Holtz, K. Petri, J. K. Joung, Engineered CRISPR prime editors with compact, untethered reverse transcriptases. Nat Biotechnol (2022), doi: 10.1038/s41587-022-01473-1.
34. L.-T. Guo, R. L. Adams, H. Wan, N. C. Huston, O. Potapova, S. Olson, C. M. Gallardo, B. R. Graveley, B. E. Torbett, A. M. Pyle, Sequencing and Structure Probing of Long RNAs Using MarathonRT: A Next-Generation Reverse Transcriptase. J Mol Biol. 432, 3338-3352 (2020).
35. H. E. Upton, L. Ferguson, M. M. Temoche-Diaz, X.-M. Liu, S. C. Pimentel, N. T. Ingolia, R. Schekman, K. Collins, Low-bias ncRNA libraries using ordered two-template relay: Serial template jumping by a modified retroelement reverse transcriptase. Proc Natl Acad Sci USA. 118, e2107900118 (2021).
36. S. Maguire, S. Guan, Rolling circle reverse transcription enables high fidelity nanopore sequencing of small RNA. PLOS One. 17, e0275471 (2022).
37. R. M. Nottingham, D. C. Wu, Y. Qin, J. Yao, S. Hunicke-Smith, A. M. Lambowitz, RNA-seq of human reference RNA samples using a thermostable group II intron reverse transcriptase. Rna. 22 (2016), pp. 597-613.
38. M. J. Shurtleff, J. Yao, Y. Qin, R. M. Nottingham, M. M. Temoche-Diaz, R. Schekman, A. M. Lambowitz, Broad role for YBX1 in defining the small noncoding RNA composition of exosomes. Proc Natl Acad Sci USA. 114, E8987-E8995 (2017).
39. L. Faucher-Giguère, A. Roy, G. Deschamps-Francoeur, S. Couture, R. M. Nottingham, A. M. Lambowitz, M. S. Scott, S. Abou Elela, High-grade ovarian cancer associated H/ACA snoRNAs promote cancer cell proliferation and survival. NAR Cancer. 4, zcab050 (2022).
40. V. Boivin, G. Deschamps-Francoeur, S. Couture, R. M. Nottingham, P. Bouchard-Bourelle, A. M. Lambowitz, M. S. Scott, S. Abou-Elela, Simultaneous sequencing of coding and noncoding RNA reveals a human transcriptome dominated by a small number of highly expressed noncoding genes. RNA. 24 (2018), pp. 950-965.
41. S. K. Deshmukh, S. K. Srivastava, T. Poosarla, D. L. Dyess, N. P. Holliday, A. P. Singh, S. Singh, Inflammation, immunosuppressive microenvironment and breast cancer: opportunities for cancer prevention and therapy. Ann Transl Med. 7, 593 (2019).
42. M. I. Love, W. Huber, S. Anders, Moderated estimation of fold change and dispersion for RNA-seq data with DESeq2. Genome Biology. 15 (2014), p. 550.
43. I. Bièche, F. Lerebours, S. Tozlu, M. Espie, M. Marty, R. Lidereau, Molecular profiling of inflammatory breast cancer: identification of a poor-prognosis gene expression signature. Clin Cancer Res. 10, 6789-6795 (2004).
44. I. Bekhouche, P. Finetti, J. Adelaïde, A. Ferrari, C. Tarpin, E. Charafe-Jauffret, C. Charpin, G. Houvenaeghel, J. Jacquemier, G. Bidaut, D. Birnbaum, P. Viens, M. Chaffanet, F. Bertucci, High-resolution comparative genomic hybridization of inflammatory breast cancer and identification of candidate genes. PLOS One. 6, e16950 (2011).
45. F. Bertucci, N. T. Ueno, P. Finetti, P. Vermeulen, A. Lucci, F. M. Robertson, M. Marsan, T. Iwamoto, S. Krishnamurthy, H. Masuda, P. Van Dam, W. A. Woodward, M. Cristofanilli, J. M. Reuben, L. Dirix, P. Viens, W. F. Symmans, D. Birnbaum, S. J. Van Laere, Gene expression profiles of inflammatory breast cancer: correlation with response to neoadjuvant chemotherapy and metastasis-free survival. Ann Oncol. 25, 358-365 (2014).
46. X. Wang, T. Semba, L. T. H. Phi, S. Chainitikun, T. Iwase, B. Lim, N. T. Ueno, Targeting Signaling Pathways in Inflammatory Breast Cancer. Cancers (Basel). 12, E2479 (2020).
47. S. Cao, J. R. Wang, S. Ji, P. Yang, Y. Dai, S. Guo, M. D. Montierth, J. P. Shen, X. Zhao, J. Chen, J. J. Lee, P. A. Guerrero, N. Spetsieris, N. Engedal, S. Taavitsainen, K. Yu, J. Livingstone, V. Bhandari, S. M. Hubert, N. C. Daw, P. A. Futreal, E. Efstathiou, B. Lim, A. Viale, J. Zhang, M. Nykter, B. A. Czerniak, P. H. Brown, C. Swanton, P. Msaouel, A. Maitra, S. Kopetz, P. Campbell, T. P. Speed, P. C. Boutros, H. Zhu, A. Urbanucci, J. Demeulemeester, P. Van Loo, W. Wang, Estimation of tumor cell total mRNA expression in 15 cancer types predicts disease progression. Nat Biotechnol. 40, 1624-1633 (2022).
48. M. Zatzman, F. Fuligni, R. Ripsman, T. Suwal, F. Comitani, L.-M. Edward, R. Denroche, G. H. Jang, F. Notta, S. Gallinger, S. P. Selvanathan, J. A. Toretsky, M. D. Hellmann, U. Tabori, A. Huang, A. Shlien, Widespread hypertranscription in aggressive human cancers. Sci Adv. 8, eabn0238 (2022).
49. S. Trop-Steinberg, Y. Azar, AP-1 Expression and its Clinical Relevance in Immune Disorders and Cancer. Am J Med Sci. 353, 474-483 (2017).
50. E. Charafe-Jauffret, C. Ginestier, F. Iovino, C. Tarpin, M. Diebel, B. Esterni, G. Houvenaeghel, J.-M. Extra, F. Bertucci, J. Jacquemier, L. Xerri, G. Dontu, G. Stassi, Y. Xiao, S. H. Barsky, D. Birnbaum, P. Viens, M. S. Wicha, Aldehyde dehydrogenase 1-positive cancer stem cells mediate metastasis and poor clinical outcome in inflammatory breast cancer. Clin Cancer Res. 16, 45-55 (2010).
51. C. M. Fillmore, C. Kuperwasser, Human breast cancer cell lines contain stem-like cells that self-renew, give rise to phenotypically diverse progeny and survive chemotherapy. Breast Cancer Res. 10, R25 (2008).
52. Y. Qi, X. Wang, X. Kong, J. Zhai, Y. Fang, X. Guan, J. Wang, Expression signatures and roles of microRNAs in inflammatory breast cancer. Cancer Cell International. 19 (2019), p. 23.
53. B. G. Debeb, L. Lacerda, S. Anfossi, P. Diagaradjane, K. Chu, A. Bambhroliya, L. Huo, C. Wei, R. A. Larson, A. R. Wolfe, W. Xu, D. L. Smith, L. Li, C. Ivan, P. K. Allen, W. Wu, G. A. Calin, S. Krishnamurthy, X. H. Zhang, T. A. Buchholz, N. T. Ueno, J. M. Reuben, W. A. Woodward, miR-141-mediated regulation of brain metastasis from breast cancer. JNCI: Journal of the National Cancer Institute. 108 (2016).
54. I. Van der Auwera, R. Limame, P. Van Dam, P. B. Vermeulen, L. Y. Dirix, S. J. Van Laere, Integrated miRNA and mRNA expression profiling of the inflammatory breast cancer subtype. British journal of cancer. 103 (2010), pp. 532-541.
55. S. Anfossi, A. Giordano, H. Gao, E. N. Cohen, S. Tin, Q. Wu, R. J. Garza, B. G. Debeb, R. H. Alvarez, V. Valero, G. N. Hortobagyi, G. A. Calin, N. T. Ueno, W. A. Woodward, J. M. Reuben, High serum miR-19a levels are associated with inflammatory breast cancer and are predictive of favorable clinical outcome in patients with metastatic HER2+ inflammatory breast cancer. PloS One. 9 (2014), p. e83113.
56. A. M. Newman, C. B. Steen, C. L. Liu, A. J. Gentles, A. A. Chaudhuri, F. Scherer, M. S. Khodadoust, M. S. Esfahani, B. A. Luca, D. Steiner, M. Diehn, A. A. Alizadeh, Determining cell type abundance and expression from bulk tissues with digital cytometry. Nat Biotechnol. 37 (2019), pp. 773-782.
57. J. Palmblad, The role of granulocytes in inflammation. Scand J Rheumatol. 13, 163-172 (1984).
58. F. Lerebours, G. Cizeron-Clairac, A. Susini, S. Vacher, E. Mouret-Fourme, C. Belichard, E. Brain, J.-L. Alberini, rique Spyratos Fré, dé, R. Lidereau, others, miRNA expression profiling of inflammatory breast cancer identifies a 5-miRNA signature predictive of breast tumor aggressiveness. International Journal of Cancer. 133 (2013), pp. 1614-1623.
59. W. Sun, W. Shen, S. Yang, F. Hu, H. Li, T.-H. Zhu, miR-223 and miR-142 attenuate hematopoietic cell proliferation, and miR-223 positively regulates miR-142 through LMO2 isoforms and CEBP-$$. Cell research. 20 (2010), pp. 1158-1169.
60. F. Aziz, The emerging role of miR-223 as novel potential diagnostic and therapeutic target for inflammatory disorders. Cellular Immunology. 303 (2016), pp. 1-6.
61. S. K. Chakrabortty, A. Prakash, G. Nechooshtan, S. Hearn, T. R. Gingeras, Extracellular vesicle-mediated transfer of processed and functional RNY5 RNA. RNA. 21, 1966-1979 (2015).
62. M. S. Protasova, T. V. Andreeva, E. I. Rogaev, Factors Regulating the Activity of LINE1 Retrotransposons. Genes (Basel). 12, 1562 (2021).
63. D. A. Flasch, Á. Macia, L. Sánchez, M. Ljungman, S. R. Heras, J. L. García-Pérez, T. E. Wilson, J. V. Moran, Genome-wide de novo L1 Retrotransposition Connects Endonuclease Activity with Replication. Cell. 177, 837-851.e28 (2019).
64. A. F. Smit, Interspersed repeats and other mementos of transposable elements in mammalian genomes. Curr Opin Genet Dev. 9, 657-663 (1999).
65. B. Tiwari, A. E. Jones, C. J. Caillet, S. Das, S. K. Royer, J. M. Abrams, p53 directly represses human LINE1 transposons. Genes Dev. 34, 1439-1451 (2020).
66. W. McKerrow, X. Wang, C. Mendez-Dorantes, P. Mita, S. Cao, M. Grivainis, L. Ding, J. LaCava, K. H. Burns, J. D. Boeke, D. Fenyö, LINE-1 expression in cancer correlates with p53 mutation, copy number alteration, and S phase checkpoint. Proc Natl Acad Sci USA. 119, e2115999119 (2022).
67. H. Tunbak, R. Enriquez-Gasca, C. H. C. Tie, P. A. Gould, P. Mlcochova, R. K. Gupta, L. Fernandes, J. Holt, A. G. van der Veen, E. Giampazolias, K. H. Burns, P. V. Maillard, H. M. Rowe, The HUSH complex is a gatekeeper of type I interferon through epigenetic regulation of LINE-1s. Nat Commun. 11, 5387 (2020).
68. K. Zhao, J. Du, Y. Peng, P. Li, S. Wang, Y. Wang, J. Hou, J. Kang, W. Zheng, S. Hua, X.-F. Yu, LINE1 contributes to autoimmunity through both RIG-I- and MDA5-mediated RNA sensing pathways. J Autoimmun. 90, 105-115 (2018).
69. S. Gatault, F. Legrand, M. Delbeke, S. Loiseau, M. Capron, Involvement of eosinophils in the anti-tumor response. Cancer Immunol Immunother. 61, 1527-1534 (2012).
70. N. Rodić, R. Sharma, R. Sharma, J. Zampella, L. Dai, M. S. Taylor, R. H. Hruban, C. A. Iacobuzio-Donahue, A. Maitra, M. S. Torbenson, M. Goggins, I.-M. Shih, A. S. Duffield, E. A. Montgomery, E. Gabrielson, G. J. Netto, T. L. Lotan, A. M. De Marzo, W. Westra, Z. A. Binder, B. A. Orr, G. L. Gallia, C. G. Eberhart, J. D. Boeke, C. R. Harris, K. H. Burns, Long interspersed element-1 protein expression is a hallmark of many human cancers. Am J Pathol. 184, 1280-1286 (2014).
71. D. Ardeljan, M. S. Taylor, D. T. Ting, K. H. Burns, The Human Long Interspersed Element-1 Retrotransposon: An Emerging Biomarker of Neoplasia. Clin Chem. 63, 816-822 (2017).
72. S. E. Singletary, C. Allred, P. Ashley, L. W. Bassett, D. Berry, K. I. Bland, P. I. Borgen, G. Clark, S. B. Edge, D. F. Hayes, L. L. Hughes, R. V. P. Hutter, M. Morrow, D. L. Page, A. Recht, R. L. Theriault, A. Thor, D. L. Weaver, H. S. Wieand, F. L. Greene, Revision of the American Joint Committee on Cancer staging system for breast cancer. J Clin Oncol. 20, 3628-3636 (2002).
73. H. Xu, J. Yao, D. C. Wu, A. M. Lambowitz, Improved TGIRT-seq methods for comprehensive transcriptome profiling with decreased adapter dimer formation and bias correction. Scientific reports. 9 (2019), pp. 1-17.
74. H. Xu, R. M. Nottingham, A. M. Lambowitz, TGIRT-seq Protocol for the Comprehensive Profiling of Coding and Non-coding RNA Biotypes in Cellular, Extracellular Vesicle, and Plasma RNAs. Bio-Protocol. 11 (2021), doi: 10.21769/BioProtoc.4239.
75. A. Kechin, U. Boyarskikh, A. Kel, M. Filipenko, cutPrimers: A New Tool for Accurate Cutting of Primers from Reads of Targeted Next Generation Sequencing. J Comput Biol. 24, 1138-1143 (2017).
76. D. Kim, J. M. Paggi, C. Park, C. Bennett, S. L. Salzberg, Graph-based genome alignment and genotyping with HISAT2 and HISAT-genotype. Nat Biotechnol. 37, 907-915 (2019).
77. H. Li, B. Handsaker, A. Wysoker, T. Fennell, J. Ruan, N. Homer, G. Marth, G. Abecasis, R. Durbin, 1000 Genome Project Data Processing Subgroup, The Sequence Alignment/Map format and SAMtools. Bioinformatics. 25, 2078-2079 (2009).
78. A. Liberzon, C. Birger, H. Thorvaldsdó ttir, M. Ghandi, J. P. Mesirov, P. Tamayo, The molecular signatures database hallmark gene set collection. Cell Systems. 1 (2015), pp. 417-425.
79. G. O. Consortium, The gene ontology resource: 20 years and still GOing strong. Nucleic Acids Research. 47 (2019), pp. D330-D338.
80. R. M. Wright, G. V. Aglyamova, E. Meyer, M. V. Matz, Gene expression associated with white syndromes in a reef building coral, Acropora hyacinthus. BMC Genomics. 16 (2015), p. 371.

1. A method of treating inflammatory breast cancer (IBC) in a subject, the method comprising the steps of:
a) obtaining a sample from the subject;
b) determining that at least one of the genes, genomic features, and/or intron-exon junction RNAs, spliced or unspliced precursor RNAs, or intron RNAs related to said gene or genomic feature listed in any of Tables 3-7, are differentially expressed or present at different levels compared to the same genes or genomic features in a healthy and/or non-IBC control individual;
c) diagnosing the subject as having IBC; and
d) treating the subject with appropriate medication specific for IBC.

2. The method of claim 1, wherein at least two or more genes, genomic features, and/or intron-exon junction RNAs, spliced or unspliced precursor RNAs, or intron RNAs related to said gene or genomic feature listed in any of Tables 3-7, are differentially regulated compared to the same genes or genomic features in a non-IBC control.

3. The method of claim 2, wherein, after step b), the two or more differentially regulated genes or genomic features are used to calculate an IBC risk score for the subject.

4. The method of claim 3, wherein the IBC risk score is compared to a cutoff value, wherein when the subject's IBC risk score is calculated to be above the cutoff value, the subject is diagnosed as having IBC and is treated accordingly.

5. The method of claim 1, wherein after a risk score is calculated, the subject is placed into a risk category based on their risk level.

6. The method of claim 5, wherein a subject with a low risk level of having IBC is treated differently than a subject with a moderate or high-risk level of having IBC.

7. The method of claim 1, wherein the gene or genes are selected from the group comprising: ADAMTS6, ADAMTSL1, ADGRV1, ANSK1B, BOLL, C6orf118, CCDC150, CHCHD6, CLSTN2, CMSS1, DGKB, DIRAS3, FAM135B, KCNH5, LUZP2, METTL15, MIOS, NADK2, NLGN1, NUP210L, PHF24, PLVAP, SENP2, SPOCK3, TRBJ1-n, TRBJ1-1, TRBJ1-2, TRBJ1-3, TRBJ1-4, TRBJ1-5, TRBJ1-6, UVRAG, WWP2, and XXYLT1.

8. The method of claim 1, wherein the non-IBC control is from one or more healthy (non-cancerous) samples.

9. The method of claim 1, wherein the non-IBC control is from one or more positive, non-IBC breast cancer samples.

10. The method of claim 1, wherein the sample from the subject is a blood, PBMC, tumor, or plasma sample.

11. The method of claim 1, wherein after step b), further testing is carried out to determine that the subject is IBC-positive.

12. The method of claim 11, wherein further testing comprises a biopsy, imaging, and/or determining one or more further biomarkers of IBC.

13. (canceled)

14. (canceled)

15. The method of claim 12, wherein said biomarkers comprise one or more of, on a nucleic acid level: activation of NF-κB pathways; higher expression levels of COX-2; inflammatory cytokines TNFα, IL-6, and/or IL-8; angiogenic factor VEGF; 1; or, on a protein level: activation of JAK/STAT pathways; elevated levels of EGFR, E-cadherin, Rho-GTPase, or translation initiation factor eIF4G1; or a previously identified gene signature for IBC; or a hybrid epithelial/mesenchymal (E/M) or partial epithelial-to-mesenchymal transition (EMT) phenotype, including both epithelial and mesenchymal properties; and/or identifying one or more of ARPC5L, HYOU1, AVEN, CHST11, TLN2, CHST3, PRKCA, COL4A4, or TNC.

16. The method claim 3, wherein the risk score is also calculated based upon the subject's past history, family history, or results of other genetic panels.

17. The method of claim 1, wherein a gene is upregulated or downregulated compared to a control.

18. The method of claim 1, wherein a genomic feature is present in greater or lesser amounts compared to a control.

19. The method of claim 18, wherein the amount of the genomic feature differs by greater than 5% compared to a control.

20. The method of claim 1, wherein RNA is detected.

21-41. (canceled)

42. A method of determining the effectiveness of a treatment for inflammatory breast cancer (IBC) in a subject, the method comprising the steps of:
a) obtaining a sample from the subject undergoing treatment;
b) determining that at least one of the genes, genomic features, and/or intron-exon junction RNAs, spliced or unspliced precursor RNAs, or intron RNAs related to said gene or genomic feature listed in any of Tables 3-7, are differentially regulated compared to the same genes or genomic features in a non-IBC control;
c) determining how the subject is responding to treatment; and
d) adjusting treatment of the subject based on results.

43-49. (canceled)

50. A kit for assessing risk level of IBC, wherein the kit comprises means for detecting two or more genes, genomic features, and/or intron-exon junction RNAs, spliced or unspliced precursor RNAs, or intron RNAs related to said gene or genomic feature listed in any of Tables 3-7.

51-55. (canceled)
